(19) United States
(12) Patent Application Publication
(10) Pub. No.: US 2025/0288670 A1
(43) Pub. Date: Sep. 18, 2025

(54) GLYCAN PROGRAMMED CELL THERAPY

(71) Applicant: Gnubiotics Sciences SA, Epalinges (CH)

(72) Inventor: Adeyemi Adesokan, Epalinges (CH)

(73) Assignee: Gnubiotics Sciences SA, Epalinges (CH)

(21) Appl. No.: 19/079,405

(22) Filed: Mar. 13, 2025

Related U.S. Application Data
(60) Provisional application No. 63/565,460, filed on Mar. 14, 2024.

(51) Int. Cl. A61K 40/11; A61K 9/00; A61K 45/06; A61P 35/00; C12N 5/00; C12N 5/0783

(52) U.S. Cl. CPC A61K 40/11; A61K 9/0019; A61K 45/06; A61P 35/00; C12N 5/0636; C12N 5/562

(57) ABSTRACT

Provided herein are methods treating cancer by administering to a recipient mammal reprogrammed T cells. The reprogrammed T cells of the disclosure is produced by a method comprising administering to the donor mammal an effective amount of a composition comprising a plurality of glycopeptides as described herein. Also provided are compositions comprising reprogrammed T cells and methods of producing reprogrammed T cells.

### CROSS-REFERENCE TO RELATED PATENT APPLICATION

This application claims the benefit under 35 U.S.C. § 119(e) of U.S. Provisional Application No. 63/565,460, filed on Mar. 14, 2024, the entire contents of which are incorporated herein by reference.

### BACKGROUND

Annually, cancer causes about 8.8 million deaths worldwide, accounting for about 15.7% of all deaths.

### SUMMARY OF THE DISCLOSURE

A common alteration present on the cells of epithelial tumors includes the glycosylation of proteins. It is estimated that 95% of all surface proteins are glycosylated and by altering the protein structure, their glycosylation pattern influences immune recognition and can mediate cancer immune evasion. The diversity of glycans is determined by the nature of their carbohydrate building blocks, how these are linked, their branching pattern, and the non-carbonate binding partner (proteins or lipids). In glycoproteins the glycans that are linked to the hydroxyl group of Serine or Threonin (0-glycans) or to the nitrogen atom of Aspargine (N-glycans). Glycans are highly tissue-, cell-, and protein-specific and are determined by the expression level of glycosyltransferases and glycosidases that orchestrate the addition and removal of pre-defined glycan building blocks to nascent and newly translated proteins. Altered glycosylation is a hallmark of epithelial tumors which amongst others includes the truncation of mucin-like O-glycan structures (e.g., the Thomsen-nouveau (Tn) antigen which consists of a single N-acetylgalactosamine (GalNAc), and the T antigen where a galactose (Gal) is added to the GalNAc residue of Tn). In epithelial tumors, these truncated T and Tn antigens are often extended through enhanced sialylation (sTn and sT, respectively). The mechanisms of how alterations in the glycosylation pattern affect tumor behavior are manifold: truncated O-glycosylation, which is often found in early-stage cancers, hampers E-cadherin function on epithelial cells, leading to a reduction in cell-cell connections, increased tissue dissociation, and epithelial-to-mesenchymal transition, which ultimately contributes to malignant transformation. Altered glycosylation of growth receptors affects their interaction with ligands to promote tumor growth and altered glycosylation on surface proteins can affect their immunogenicity, suppress anti-tumor immunity, and promote immune escape. Thus, overall alterations in the glycosylation pattern are associated with negative clinical outcome. It is estimated that 95% of all surface proteins are glycosylated and by altering the protein structure, aberrant glycosylation influences immune recognition and can promote cancer immune evasion. Many of these glycan epitopes were found to modulate innate and adaptive immune responses via interaction with lectin receptors on immune cells suggesting that they might be utilized as novel therapeutic targets. The low immunogenicity of glycans and the induction of largely T cell-independent IgM-driven immune responses to carbohydrate antigens, however, make the use of glycans for classical immunization approaches challenging. Moreover, targeting single glycopeptide epitopes carrying sTn or Tn antigens lacked clinical efficacy, thus novel approaches are needed.

Some aspects of the present disclosure are directed to a method for treating cancer in a recipient mammal in need thereof comprising, or alternatively consisting essentially of, or yet further consisting of administering to the recipient mammal an effective amount of T cells isolated from a donor mammal, wherein the T cells were prepared by a method comprising, or alternatively consisting essentially of, or yet further consisting of administering to the donor mammal an effective amount of a composition comprising, or alternatively consisting essentially of, or yet further consisting of a plurality of glycopeptides, wherein at least 90% of the plurality of glycopeptides in the composition are less than 2 KDa in size, and wherein the plurality of glycopeptides comprises, or alternatively consists essentially of, or yet further consists of the following oligosaccharide structures: Fucα1-2Galβ1-3GalNAc, Galβ1-3(GlcNAcβ1-6)GalNAc, Galβ1-3(6SGlcNAcβ1-6)GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, Galβ1-3(Galβ1-4GlcNAcβ1-6)GalNAc, Fucα1-2Galβ1-3(6S-GlcNAcβ1-6)GalNAc, Galβ1-3(Galα1-3Galβ1-4GlcNAcβ1-6)GalNAc, and NeuAcα2-3Galβ1-3[(6S)GlcNAcβ1-6]GalNAc; thereby treating the cancer in the recipient mammal.

In some embodiments, the T cells are autologous or allogeneic to the recipient mammal, or wherein the T cells are HLA1-deficient T cells or TCR-deficient T cells.

In some embodiments, the composition is administered to the donor mammal for 28 days or more before isolating the T cells.

In some embodiments, the plurality of glycopeptides is derived from porcine gastrointestinal mucins, and wherein the composition is obtained without subjecting the porcine gastrointestinal mucins to conditions or reagents that cause release of oligosaccharides from glycopeptides.

In some embodiments, the composition comprises, or alternatively consists essentially of, or yet further consists of between about 0.5-1% Fucα1-2Galβ1-3GalNAc containing glycopeptides, between about 0.5-1% Galβ1-3(GlcNAcβ1-6)GalNAc containing glycopeptides, between about 3-5% Galβ1-3(6SGlcNAcβ1-6)GalNAc containing glycopeptides, between about 1-3% Galβ1-3(NeuAcα2-6)GalNAc, between about 1-3% NeuAcα2-3Galβ1-3GalNAc containing glycopeptides, between about 1-3% Galβ1-3(NeuGcα2-6)GalNAc containing glycopeptides, between about 1-3% NeuGcα2-3Galβ1-3GalNAc containing glycopeptides, between about 0.5-1% Galβ1-3(Galβ1-4GlcNAcβ1-6)GalNAc containing glycopeptides, between about 3-5% Fucα1-2Galβ1-3(6S-GlcNAcβ1-6)GalNAc containing glycopeptides, between about 0.5-1% Galβ1-3(Galα1-3Galβ1-4GlcNAcβ1-6)GalNAc containing glycopeptides, and between about 0.5-1% NeuAcα2-3Galβ1-3[(6S)GlcNAcβ1-6]GalNAc containing glycopeptides by weight of the total composition.

In some embodiments, the composition has a free glycan content of less than 0.1% by weight.

In some embodiments, the composition contains less than 5% of insoluble particles having a diameter greater than 7 μm.

In some embodiments, the glycopeptides in the plurality of glycopeptides are less than 15 amino acids.

In some embodiments, the composition is administered to the donor mammal orally or rectally.

In some embodiments, the cancer is selected from melanoma, breast cancer, lung cancer or colorectal cancer (CRC). In some embodiments, the cancer is stage 3 or 4 cancer. In some embodiments, the cancer is resistant to checkpoint inhibitor therapy.

In some embodiments, the method further comprises, or alternatively consists essentially of, or yet further consists of administering to the recipient mammal an immune checkpoint inhibitor before, after, or simultaneously with the isolated T cells.

Another aspect of the disclosure is directed to a composition comprising, or alternatively consisting essentially of, or yet further consisting of isolated T cells and a cryoprotective agent, wherein the T cells were prepared by a method comprising, or alternatively consisting essentially of, or yet further consisting of administering to a donor mammal an effective amount of a composition comprising, or alternatively consisting essentially of, or yet further consisting of a plurality of glycopeptides, wherein at least 90% of the plurality of glycopeptides in the composition are less than 2 KDa in size, and wherein the plurality of glycopeptides comprises, or alternatively consists essentially of, or yet further consists of the following oligosaccharide structures: Fucα1-2Galβ1-3GalNAc, Galβ1-3(GlcNAcβ1-6)GalNAc, Galβ1-3(6SGlcNAcβ1-6)GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, Galβ1-3(Galβ1-4GlcNAcβ1-6)GalNAc, Fucα1-2Galβ1-3(6S-GlcNAcβ1-6)GalNAc, Galβ1-3(Galα1-3Galβ1-4GlcNAcβ1-6)GalNAc, and NeuAcα2-3Galβ1-3[(6S)GlcNAcβ1-6]GalNAc.

In some embodiments, the composition is administered to the donor mammal for 28 days or more before isolating the T cells, and optionally wherein the T cells are HLA1-deficient T cells or TCR-deficient T cells.

In some embodiments, the glycopeptides are derived from porcine gastrointestinal mucins, and wherein the composition is obtained without subjecting the porcine gastrointestinal mucins to conditions or reagents that cause complete release of oligosaccharides from glycopeptides.

In some embodiments, the composition comprises, or alternatively consists essentially of, or yet further consists of between about 0.5-1% Fucα1-2Galβ1-3GalNAc containing glycopeptides, between about 0.5-1% Galβ1-3(GlcNAcβ1-6)GalNAc containing glycopeptides, between about 3-5% Galβ1-3(6SGlcNAcβ1-6)GalNAc containing glycopeptides, between about 1-3% Galβ1-3(NeuAcα2-6)GalNAc, between about 1-3% NeuAcα2-3Galβ1-3GalNAc containing glycopeptides, between about 1-3% Galβ1-3(NeuGcα2-6)GalNAc containing glycopeptides, between about 1-3% NeuGcα2-3Galβ1-3GalNAc containing glycopeptides, between about 0.5-1% Galβ1-3(Galβ1-4GlcNAcβ1-6)GalNAc containing glycopeptides, between about 3-5% Fucα1-2Galβ1-3(6S-GlcNAcβ1-6)GalNAc containing glycopeptides, between about 0.5-1% Galβ1-3(Galα1-3Galβ1-4GlcNAcβ1-6)GalNAc containing glycopeptides, and between about 0.5-1% NeuAcα2-3Galβ1-3[(6S)GlcNAcβ1-6]GalNAc containing glycopeptides by weight of the total composition.

In some embodiments, the composition has a free glycan content of less than 0.1% by weight.

In some embodiments, the composition contains less than 5% of insoluble particles having a diameter greater than 7 μm.

In some embodiments, the glycopeptides in the plurality of glycopeptides are less than 15 amino acids.

In some embodiments, the composition is administered to the donor mammal orally or rectally.

Another aspect of the disclosure is directed to a method of preparing reprogrammed T cells comprising, or alternatively consisting essentially of, or yet further consisting of: administering to a donor animal (e.g., a donor mammal) an effective amount of a composition comprising, or alternatively consisting essentially of, or yet further consisting of a plurality of glycopeptides, wherein at least 90% of the plurality of glycopeptides in the composition are less than 2 KDa in size, and wherein the plurality of glycopeptides comprises, or alternatively consists essentially of, or yet further consists of the following oligosaccharide structures: Fucα1-2Galβ1-3GalNAc, Galβ1-3(GlcNAcβ1-6)GalNAc, Galβ1-3(6SGlcNAcβ1-6)GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, Galβ1-3(Galβ1-4GlcNAcβ1-6)GalNAc, Fucα1-2Galβ1-3(6S-GlcNAcβ1-6)GalNAc, Galβ1-3(Galα1-3Galβ1-4GlcNAcβ1-6)GalNAc, and NeuAcα2-3Galβ1-3[(6S)GlcNAcβ1-6]GalNAc; and isolating the T cells from the donor animal (e.g. a donor mammal).

In some embodiments, the method further comprises, or alternatively consists essentially of, or yet further consists of cryopreserving the isolated T cells.

In some embodiments, the composition is administered to the donor mammal for 28 days or more before isolating the T cells.

In some embodiments, the glycopeptides were derived from porcine gastrointestinal mucins, and wherein the composition is obtained without subjecting the porcine gastrointestinal mucins or the partially purified fraction thereof to conditions or reagents that cause complete release of oligosaccharides from glycopeptides.

In some embodiments, the composition comprises, or alternatively consists essentially of, or yet further consists of between about 0.5-1% Fucα1-2Galβ1-3GalNAc containing glycopeptides, between about 0.5-1% Galβ1-3(GlcNAcβ1-6)GalNAc containing glycopeptides, between about 3-5% Galβ1-3(6SGlcNAcβ1-6)GalNAc containing glycopeptides, between about 1-3% Galβ1-3(NeuAcα2-6)GalNAc, between about 1-3% NeuAcα2-3Galβ1-3GalNAc containing glycopeptides, between about 1-3% Galβ1-3(NeuGcα2-6)GalNAc containing glycopeptides, between about 1-3% NeuGcα2-3Galβ1-3GalNAc containing glycopeptides, between about 0.5-1% Galβ1-3(Galβ1-4GlcNAcβ1-6)GalNAc containing glycopeptides, between about 3-5% Fucα1-2Galβ1-3(6S-GlcNAcβ1-6)GalNAc containing glycopeptides, between about 0.5-1% Galβ1-3(Galα1-3Galβ1-4GlcNAcβ1-6)GalNAc containing glycopeptides, and between about 0.5-1% NeuAcα2-3Galβ1-3[(6S)GlcNAcβ1-6]GalNAc containing glycopeptides by weight of the total composition.

In some embodiments, the composition has a free glycan content of less than 0.1% by weight.

In some embodiments, the composition contains less than 5% of insoluble particles having a diameter greater than 7 μm.

In some embodiments, the glycopeptides in the plurality of glycopeptides are less than 15 amino acids.

In some embodiments, the T cells are HLA1-deficient T cells or TCR-deficient T cells.

The above discussed, and many other features and attendant advantages of the present inventions will become better understood by reference to the following detailed description of the invention.

### BRIEF DESCRIPTION OF THE DRAWINGS

FIG. 1. A schematic of diafiltration for the manufacture of the compositions comprising glycopeptides of the instant disclosure.

FIGS. 2A-2B. (A) Glycan core structures found in the compositions of the instant disclosure. (B) Glycan core structures common to the compositions of the instant disclosure and tumors.

FIGS. 3A-3G. Supplementation of GPC reduces tumor growth. (A) Schematic overview of the experimental layout. 9-12-week-old C57BL/6 mice were injected on each flank with 300,000 MC38 cells and treated with 3% GPC in the drinking water starting two weeks before (prophylactic, (B)-(D) or 6 days after (therapeutic approach, (E)-(G)) tumor cell injection. On day 6, day 9 and day 12 post tumor cell injection, the mice were injected with an isotype control or an anti-PD-1 antibody.

FIGS. 4A-4G. GPC administration reduces tumor growth in a melanoma and a breast cancer model. (A) Schematic overview of the experimental layout. 9-12-week-old C57BL/6 mice were injected on each flank with (B)-(D) 300,000 YUMM1.7 melanoma cells or (E)-(G) 500,000 4T1 breast cancer cells. Cells were treated with 3% GPC in the drinking water starting two weeks before or 6 days after tumor cell injection. (C) and (F) representative pictures. (D) and (G) tumor weight of tumors isolated on day 15 after tumor cell injection.

FIGS. 5A-5D. GPC administration reduces tumor growth in an orthotopic cecum injection model for CRC. 300,000 MC38 cells were injected into the cecum wall of 9-12-week-old C57BL/6 mice. The mice were treated with 3% GPC in the drinking water starting 6 days after tumor cell injection. On day 6, day 9 and day 12 post tumor cell injection, the mice were injected with an isotype control or an anti-PD-1 antibody. (A) Schematic overview of the experimental layout. (B) representative pictures, (C) tumor volume and (D) tumor weight of tumors on day 15 after tumor cell injection.

FIGS. 6A-6F. GPC administration promotes T cell activation in tumors. 9-12-week-old C57BL/6 mice were injected on each flank with 300,000 MC38 cells and treated with 3% GPC in the drinking water starting 6 days after tumor cell injection. On day 6, day 9 and day 12 post tumor cell injection, the mice were injected with an isotype control or an anti-PD-1 antibody. 15 days after tumor cell injection, tumors were harvested and the tissue analyzed for cytokine levels using a multiplex cytokine assay for the indicated cytokines; relative abundance of (A) macrophages among myeloid cells, (B) IL-12+ macrophages, (C) T cells, (D) CD8+ T cells using flow cytometry; (E) abundance of CD8+ cells using IHC; and (F) for the relative abundance of Perforin+/granzymeB+ and IFNγ+ CD8+ T cells using flow cytometry. H+I: Tumors were harvested and single cell suspensions either left untreated or re-stimulated with GPC or PMA+Inonomycin for 6 h.

FIGS. 7A-7H. GPC administration promotes T cell activation in the prophylactic approach. 9-12-week-old C57BL/6 mice were injected on each flank with (A)-(D) 300,000 MC38 cells or (E)-(H) YUMM1.7 melanoma cells and treated with 3% GPC in the drinking water starting two weeks before tumor cell injection. On day 6, day 9 and day 12 post tumor cell injection, the mice were injected with an isotype control or an anti-PD-1 antibody. 15 days after tumor cell injection, tumors were harvested and analyzed for the indicated cell populations using flow cytometry.

FIGS. 8A-8B. GPC administration promotes apoptosis in the tumor tissue but has no effect on cell proliferation. 9-12-week-old C57BL/6 mice were injected on each flank with 300,000 MC38 cells and treated with 3% GPC in the drinking water starting 6 days after tumor cell injection. On day 6, day 9 and day 12 post tumor cell injection, the mice were injected with an isotype control or an anti-PD-1 antibody. On day 15, the tumors were harvested and stained with hematoxylin and Eosin (H&E), with an antibody against cleaved caspase 3 (cl. casp3), or an antibody against Ki67. (A) The number of cleaved Casp3 positive cells per high magnification field. (B) The number of Ki67 positive cells per high magnification field.

FIGS. 9A-9C. (A) Schematic overview of the experimental set up; Flow cytometry for (B) CD69+ and (C) IFNγ+ cells among CD8+ T cells.

FIG. 10A-10E. Adoptive T cell transfer study. 9-12-week-old C57BL/6 mice were injected on each flank with 300,000 MC38 cells and treated with 3% GPC in the drinking water starting 6 days after tumor cell injection. On day 6, day 9 and day 12 post tumor cell injection, the mice were injected with an isotype control or an anti-PD-1 antibody. 15 days after tumor cell injection, tumors were harvested, and T cells isolated from the tumor tissue. The T cells were expanded for one week and injected i.p. into C547BL/6 recipient mice that were injected on each flank with 300,000 MC38 cells on the same day. Mice that received T cells from the spleen of non-tumor bearing mice and mice that did not receive T cells served as controls. (A) Schematic overview of the experimental layout. (B) Tumor growth over time, (C) representative pictures, (D) volume and (E) weight of tumors isolated from T cell recipient mice on day 15 after tumor cell injection.

### DETAILED DESCRIPTION OF THE DISCLOSURE

### Definitions

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more nucleic acids, polypeptides, cells, species or types of organism, disorders, subjects, or combinations thereof, can be excluded.

Where the claims or description relate to a composition of matter, e.g., a nucleic acid, polypeptide, or cell, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

A “composition” is intended to mean a combination of active agent and another compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A composition as disclosed herein can be a pharmaceutical composition. A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

As used herein, the term “excipient” refers to a natural or synthetic substance formulated alongside the active ingredient of a medication, included for the purpose of long-term stabilization, bulking up solid formulations, or to confer a therapeutic enhancement on the active ingredient in the final dosage form, such as facilitating drug absorption, reducing viscosity, or enhancing solubility.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

An “effective amount” is an amount necessary to achieve the desired therapeutic or diagnostic result and will vary with the disease and subject or patient to be treated. Effective amounts are determined using methods known in the art and as described herein.

The expression “gastrointestinal tract mucins” encompasses any natural source of mucin from which glycans and glycopeptides can be extracted, suitable for mammalian nutrition or pharmaceutical use. Typical sources of gastrointestinal tract mucins are extracts from gastrointestinal tract, in particular from porcine sources or from bovine sources. Commercial sources for gastrointestinal tract mucins include Biofac A/S (Kastrup, Denmark), Zhongshi Duqing (Heze, China), Shenzhen Taier Biotechnology Co., LTD (Shenzhen, China), and Dongying Tiandong Pharmaceutical Co. (Shandong, China). In some embodiments, the gastrointestinal tract mucins are from porcine gastric mucus.

The expression “subject” refers to mammals. For examples, mammals contemplated by the present invention include human, primates, domesticated animals such as cattle, sheep, pigs, horses, rodents, cats, dogs, and other pets. In some embodiments, the subject is a human. In some embodiments, the subject may be an infant (1 year old or less for a human), a toddler (3 years old or less for a human), a child, a young adult, an adult or a geriatric. In some embodiments, the infant is a newborn. The subject may be male or female. In some embodiments, the subject is female and of child-bearing age.

The term “glycoprotein” refers to proteins linked to oligosaccharides, e.g., proteins either N-linked or O-linked to oligosaccharides, and having a molecular weight of more than about 5 KDa.

The term “glycopeptide” refers to peptides linked to oligosaccharides, e.g. peptides either N-linked or O-linked to oligosaccharides, and having a molecular weight of less than about 5 KDa. Methods of determining molecular weight of glycopeptides and glycoproteins are known in the art and are not limited. In some embodiments, the molecular weight of glycopeptides and glycoproteins are determined by size exclusion chromatography.

The term “glycan” as used herein refers to an oligosaccharide. The term “free glycan” is synonymous with the term “free oligosaccharide,” as also used herein.

In some embodiments, peptides are defined as having a molecular weight of less than about 5 KDa. In some embodiments, the term peptides include glycopeptides. In some embodiments, proteins are defined as having a molecular weight of more than about 5 KDa. In some embodiments, the term proteins include glycoproteins.

As used herein, “a partially purified fraction” of gastrointestinal tract mucins comprises, or alternatively consists essentially of, or yet further consists of at least about 40%, at least about 50%, at least about 60%, at least about 70%, at least about 80%, at least about 90%, at least about 92.5%, at least about 95%, at least about 97.5%, at least about 98%, at least about 99%, or at least about 99.5% of the protein- and peptide-glycans present in un-purified gastrointestinal tract mucins. In some embodiments, the mucins or partially purified fraction thereof has been subject to an acid treatment.

The terms “treating” and “treatment” refer to administering to a subject an effective amount of a composition so that the subject experiences a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. In another aspect, the term “treatment” excludes prophylaxis. Alternatively, treatment is “effective” if the progression of a disease is reduced or halted. “Treatment” can also mean prolonging survival as compared to expected survival if not receiving treatment.

The term “chemotherapy” refers to administration of any genotoxic agent (e.g., DNA damaging agent), including conventional or non-conventional chemotherapeutic agents, for the treatment or prevention of cancer. Chemotherapeutic agents include agents that have been modified, (e.g., fused to antibodies or other targeting agents). Examples of chemotherapeutic agents include, but are not limited to, platinum compounds (e.g., cisplatin, carboplatin, oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, bendamustine, mitomycin C), antitumor antibiotics (e.g., daunorubicin, doxorubicin, idarubicin, epirubicin, mitoxantrone, bleomycin, plicamycin, dactinomycin), taxanes (e.g., paclitaxel, /raZ>-paclitaxel and docetaxel), antimetabolites (e.g.: 5-fluorouracil, cytarabine, premetrexed, thioguanine, floxuridine, capecitabine, and methotrexate), nucleoside analogues (e.g., fludarabine, clofarabine, cladribine, pentostatin, nelarabine, gemcitabine, 5-flurouracil), topoisomerase inhibitors (e.g., topotecan, irinotecan, SN-38, CPT-11), hypomethylating agents (e.g., azacitidine and decitabine), proteasome inhibitors (e.g., bortezomib), epipodophyllotoxins (e.g., etoposide and teniposide), DNA synthesis inhibitors (e.g., hydroxyurea), and vinca alkaloids (e.g., vincristine, vindesine, vinorelbine, and vinblastine). Chemotherapeutic agents include DNA intercalating agents (e.g, pyrrol obenzodiazepines).

The terms, “decrease”, “reduced”, “reduction”, “decrease”, and “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase”, “enhance” or “activate” are all used herein to generally mean an increase by a statically significant amount; for the avoidance of any doubt, the terms “increased”, “increase”, “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two-standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

Several classes of checkpoint inhibitors/regulators (immune checkpoint inhibitors “ICI”) are known in the art, including lymphocyte activation gene-3 (LAG-3), T cell immunoglobulin and ITIM domain (TIGIT), T cell immunoglobulin and mucin-domain containing-3 (TIM-3), V-domain immunoglobulin suppressor of T cell activation (VISTA), B7 homolog 3 protein (B7-H3), inducible T cell costimulatory (ICOS) and B and T lymphocyte attenuator (BTLA), anti-cytotoxic T lymphocyte associated antigen-4 (CTLA-4) as well as inhibitors of CTLA-4, programmed death 1 (PD-1; also referred to herein as PD1), and programmed death ligand-1 (PD-L1).

The latter 3 classes of checkpoint inhibitors, CTLA-4, PD-1 and PD-L1 inhibitors, have contributed several medically relevant drugs such as monoclonal antibody (mAb) inhibitors. Example of anti-CTLA-4 inhibitory mAb is ipilimumab (approved globally). Example of anti-PD-L1 inhibitory mAbs are atezolizumab, avelumab and durvalumab (approved globally). Non-limiting examples of anti-PD1 inhibitor mAbs include pembrolizumab and nivolumab (approved globally); sintilimab, tislelizumab, toripalimab, and camrelizumab (approved in China); geptanolimab serplulimab zimberelimab cemiplimab, dostarlimab, prolgolimab, balstilimab, penpulimab, retifanlimab, cadonilimab, pucotenlimab, sasanlimab, and cetrelimab.

Checkpoint inhibitory mAbs of distinct subclasses can be combined in a distinct modality (e.g. ipilimumab+nivolumab), or combined individually with chemotherapy, biologic therapies, anti-angiogenic therapies such as VEGF inhibitors, anti-TGFβs, cell therapies, mRNA therapies and so on.

As used herein, the phrase “GPC composition” refers to a composition prepared by the methods described in Example 1. Briefly, a GPC a composition is produced by a method comprising: (a) stabilizing gastrointestinal mucin from intestine of an animal at pH 5.0; (b) desalinating the stabilized mucin using dialysis; (c) concentrating the desalinated mucin; (d) subjecting the concentrate from (d) to diafiltration; thereby producing the composition. In some embodiments, the concentrating in (c) is achieved by evaporation with a rotary evaporator at least 80° C. In some embodiments, the effective amount of the composition is about 0.2-0.8 grams per kilogram of the subject. In some embodiments, the method further comprises, or alternatively consists essentially of, or yet further consists of administering an immune checkpoint inhibitor before, after, or simultaneously with the composition comprising glycopeptides.

In some embodiments, the GPC composition comprises, or alternatively consists essentially of, or yet further consists of at least one glycopeptide-bound oligosaccharide having a general formulae selected from Hex1HexNAc1, HexNAc2, Hex1HexNAc1Sul, Hex1NAc2Sul1, NeuAc1HexNAc1, NeuGc1HexNAc1, Hex1HexNAc1deHex1, Hex2HexNAc1, Hex1HexNAc2, HexNAc3, Hex1HexNAc2Sul1, NeuAc1Hex1HexNAc1, NeuGc1Hex1HexNAc1, Hex2HexNAc1deHex1, Hex1NAc3Sul1, NeuAc1HexNAc2, NeuGc1HexNAc2, Hex1HexNAc2deHex1, Hex2HexNAc2, Hex1HexNAc3, Hex1HexNAc2deHex1Sul1, NeuAc1Hex1HexNAc1deHex1, Hex2HexNAcSul1, NeuGc1Hex1HexNAc1deHex1, Hex1HexNAc3Sul1, NeuAc1Hex1HexNAc2, NeuGc1Hex1HexNAc2, Hex2HexNAc2deHex1, Hex3HexNAc2, Hex1HexNAc3deHex1, Hex2HexNAc3, NeuAc1Hex1HexNAc2Sul1, NeuAc2Hex1HexNAc1, NeuGc1Hex1HexNAc2Sul1, Hex2HexNAc2deHex1Sul1, NeuAc1NeuGc1Hex1HexNAc1, NeuGc2Hex1HexNAc1, Hex1HexNAc3deHex1Sul1, NeuAc1Hex1HexNAc2deHex1, Hex2HexNAc3Sul1, NeuGc1Hex1HexNAc2deHex1, NeuAc1Hex2HexNAc2, Hex2HexNAc2deHex2, NeuGc1Hex2HexNAc2, Hex2HexNAc3deHex1, NeuAc1Hex2HexNAc2Sul1, Hex2HexNAc2deHex2Sul1, NeuGc1Hex2HexNAc2Sul1, Hex1HexNAc4deHex1, NeuAc1Hex1HexNAc3Sul1, Hex1HexNAc3deHex2Sul1, NeuAc2Hex1HexNAc2, NeuGc1Hex1HexNAc3Sul1, Hex2HexNAc3deHex1Sul1, NeuAc1Hex2HexNAc2deHex1, NeuGc2Hex1HexNAc2, NeuGc1Hex2HexNAc2deHex1, NeuGc1Hex3HexNAc2, Hex1HexNAc4deHex1Sul1, NeuAc1Hex1HexNAc3deHex1, Hex2HexNAc3deHex2 NeuGc1Hex2HexNAc3, NeuAc1Hex2HexNAc2deHex1Sul1, Hex2HexNAc3deHex2Sul1, NeuAc1Hex2HexNAc3deHex1Sul1, NeuGc1Hex2HexNAc3deHex1Sul1, Hex2HexNAc4deHex2Sul1, Hex2HexNAc5deHex2Sul1, NeuAc1Hex5HexNAc4deHex1, and NeuAc1Hex4HexNAc4deHex2Sul1.

In some embodiments, the GPC composition comprises, or alternatively consists essentially of, or yet further consists of glycopeptide-bound oligosaccharides having at least 10, at least 20, at least, 30, at least 40, at least 45, at least 50, at least 55, at least 60, at least 65, (e.g., at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65) or all of the general formulae listed above. In some embodiments, the GPC composition comprises, or alternatively consists essentially of, or yet further consists of at least 10 of the general formulae listed above, and each formula is present in the composition between 0.1% and 10% (e.g., 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%) of all glycopeptide-bound oligosaccharides in the composition.

In some embodiments, the GPC composition comprises, or alternatively consists essentially of, or yet further consists of at least 10 glycopeptide-bound oligosaccharides having a structure selected from Galβ1-3GalNAc, GalNAcα1-3GalNAc, GlcNAcβ1-6GalNAc, 3SGalβ1-3GalNAc, 6SGlcNAcβ1-3GalNAc, 6SGlcNAcβ1-6GalNAc, NeuAcα2-6GalNAc, NeuGcα2-6GalNAc, Fucα1-2(GalNAcα1-3)Gal, Fucα1-2Galβ1-3GalNAc, Fucα1-2Galβ1-4GlcNAc, Galβ1-4GlcNAcβ1-3Gal, Galβ1-3(GlcNAcβ1-6)GalNAc, Galβ1-4GlcNAcβ1-3GalNAc, GlcNAcβ1-3(GlcNAcβ1-6)GalNAc, Galβ1-3(6SGlcNAcβ1-6)GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, Fucα1-2Galβ-4GlcNAcβ1-3Gal, 6SGlcNAcβ1-3(GlcNAcβ1-6)GalNAc, GlcNAcβ1-3(6S-GlcNAcβ1-6)GalNAc, GalNAcα1-3(NeuAcα2-6)GalNAc, GlcNAcβ1-3(NeuAcα2-6)GalNAc, GalNAcα1-3(NeuGcα2-6)GalNAc, GlcNAcβ1-3(NeuGcα2-6)GalNAc, Fucα1-2(GalNAcα1-3)Galβ1-3GalNAc, Fucα1-2Galβ1-3(GlcNAcβ1-6)GalNAc, Fucα1-2Galβ1-3GlcNAcβ1-3GalNAc, Galβ1-3(Galβ1-4GlcNAcβ1-6)GalNAc, Galβ1-3GlcNAcβ1-3Galβ1-3GalNAc, GlcNAcβ1-3(Galβ1-4GlcNAcβ1-6)GalNAc, Fucα1-2Galβ1-3(6S-GlcNAcβ1-6)GalNAc, Fucα1-2Galβ1-3(NeuAcα2-6)GalNAc, Galβ1-3[Galβ1-4(6S)GlcNAcβ1-6]GalNAc, Fucα1-2Galβ1-3(NeuGcα2-6)GalNAc, Galβ1-4GlcNAcβ1-3(6SGlcNAcβ1-6)GalNAc, GlcNAcα1-4Galβ1-3[(6S)GlcNAcβ1-6]GalNAc, GlcNAcβ1-3[Galβ1-4(6S)GlcNAcβ1-6]GalNAc, GlcNAca1-4Galβ1-3(NeuAcα2-6)GalNAc, NeuAcα2-3(GalNAcβ1-4)Galβ1-3GalNAc, GalNAcβ1-4(NeuGcα2-3)Galβ1-3GalNAc, NeuGcα2-3Galβ1-3(GlcNAcβ1-6)GalNAc, Fucα1-2Galβ1-3(Galβ1-4GlcNAcβ1-6)GalNAc, Galβ1-3(Fucα1-2Galβ1-4GlcNAcβ1-6)GalNAc, Galα1-3Galβ1-4GlcNAcβ1-3Galβ1-3GalNAc, Galβ1-3(Galα1-3Galβ1-4GlcNAcβ1-6)GalNAc, Fucα1-2(GalNAcα1-3)Galβ1-4GlcNAcβ1-3GalNAc, GlcNAcβ1-3(Fucα1-2Galβ1-4GlcNAcβ1-6)GalNAc, GlcNAcα1-4Galβ1-3(Galβ1-4GlcNAcβ1-6)GalNAc, NeuAcα2-3Galβ1-3[(6S)GlcNAcβ1-6]GalNAc, NeuAcα2-3Galβ1-3(NeuAcα2-6)GalNAc, NeuGcα2-6Galβ1-3(6SGlcNAcβ1-6)GalNAc, Fucα1-2Galβ1-3[Galβ1-4(6S)GlcNAcβ1-6]GalNAc, Galβ1-3[Fucα1-2Galβ1-4(6S)GlcNAcβ1-6]GalNAc, NeuAcα2-3Galβ1-3(NeuGcα2-6)GalNAc, NeuGcα2-3Galβ1-3(NeuGcα2-6)GalNAc, Fucα1-2Galβ1-4(6S)GlcNAcβ1-3(GlcNAcβ1-6)GalNAc, Fucα1-2Galβ1-4GlcNAcβ-3(6SGlcNAcβ1-6)GalNAc, GalNAcα1-3(Fucα1-2)Galβ1-3(6SGlcNAcβ1-6)GalNAc, GlcNAcβ1-3[Fucα1-2Galβ1-4(6S)GlcNAcβ1-6]GalNAc, Fucα1-2Galβ1-4GlcNAcβ1-3(NeuAcα2-6)GalNAc, Galβ1-4GlcNAcβ1-3[Galβ1-4(6S)GlcNAcβ1-6]GalNAc, Fucα1-2Galβ1-4GlcNAcβ1-3(NeuGcα2-6)GalNAc, NeuAcα2-3Galβ1-3(Galβ1-4GlcNAcβ1-6)GalNAc, Fucα1-2Galβ1-3(Fucα1-2Galβ1-4GlcNAcβ1-6)GalNAc, NeuGcα2-3Galβ1-3(Galβ1-4GlcNAcβ1-6)GalNAc, Galβ1-3[Fucα1-2(GalNAcα1-3)Galβ1-4GlcNAcβ1-6]GalNAc, NeuAcα2-3Galβ1-3[Galβ1-4(6S)GlcNAcβ1-6]GalNAc, Fucα1-2Galβ1-3[Fucα1-2Galβ1-4(6S)GlcNAcβ1-6]GalNAc, NeuGcα2-3Galβ1-3[Galβ1-4(6S)GlcNAcβ1-6]GalNAc, GlcNAcβ1-3[GalNAcα1-3(Fucα1-2)Galβ1-3GlcNAcβ1-6]GalNAc, NeuAcα2-3(GalNAcβ1-4)Galβ1-3(6SGlcNAcβ1-6)GalNAc, 6SGlcNAcβ1-3[Fucα1-2Galβ1-(Fucα1-)GlcNAcβ1-6]GalNAc, NeuAcα2-3(GalNAcβ1-4)Galβ1-3(NeuAcα2-6)GalNAc, GlcNAcβ1-3[NeuGcα2-3Galβ1-4(6S)GlcNAcβ1-6]GalNAc, Fucα1-2Galβ1-4GlcNAcβ1-3[Galβ1-4(6S)GlcNAcβ1-6]GalNAc, Galβ1-3[GalNAcα1-3(Fucα1-2)Galβ1-4(6S)GlcNAcβ1-6]GalNAc, Galβ1-3GlcNAcβ1-3[Fucα1-2Galβ1-4(6S)GlcNAcβ1-6]GalNAc, Galβ1-4GlcNAcβ1-3[Fucα1-2Galβ1-3(6S)GlcNAcβ1-6]GalNAc, NeuAcα2-3Galβ1-3(Fucα1-2Galβ1-4GlcNAcβ1-6)GalNAc, NeuGcα2-3(GalNAcβ1-4)Galβ1-3(NeuGcα2-6)GalNAc, NeuGcα2-3Galβ1-3 (Fucα1-2Galβ1-4GlcNAcβ1-6)GalNAc, Galα1-3Galβ1-3(NeuGcα2-3Galβ1-4GlcNAcβ1-6)GalNAc, GalNAcα1-3(Fucα1-2)Galβ1-3GlcNAcβ1-3(6SGlcNAcβ1-6)GalNAc, GlcNAcβ1-3[GalNAcα1-3(Fucα1-2)Galβ1-(6S)GlcNAcβ1-6]GalNAc, GlcNAcα1-4(Fucα1-2)Galβ1-4GlcNAcβ1-3(NeuAcα2-6)GalNAc, Fucα1-2Galβ1-4GlcNAcβ1-3(Fucα1-2Galβ1-4GlcNAcβ1-6)GalNAc, Galβ1-(Fuc)GlcNAcβ1-3(Fucα1-2Galβ1-4GlcNAcβ1-6)GalNAc, NeuGcα2-3Galβ1-3(GlcNAcα1-4Galβ1-4GlcNAcβ1-6)GalNAc, NeuAcα2-3Galβ1-3[Fucα1-2Galβ1-4(6S)GlcNAcβ1-6]GalNAc, Fucα1-2Galβ1-3[GalNAcα1-3(Fucα1-2)Galβ1-3(6S)GlcNAcβ1-6]GalNAc, Fucα1-2Galβ1-3[GalNAcα1-3(Fucα1-2)Galβ1-4(6S)GlcNAcβ1-6]GalNAc, Fucα1-2Galβ1-4GlcNAcβ1-3[Fucα1-2Galβ1-4(6S)GlcNAcβ1-6]GalNAc, NeuAcα2-3Galβ1-3[GalNAcα1-3(Fucα1-2)Gal-(6S)GlcNAcβ1-6]GalNAc, GlcNAcα1-4Galβ1-3[NeuGcα2-6Gal-(Fuc)(6S)GlcNAcβ1-6]GalNAc, GlcNAcα1-4(Fucα1-2)GlcNAcβ1-3[Fucα1-2)Galβ1-4(6S)GlcNAcβ1-6]GalNAc, GalNAcα1-3(Fucα1-2)Galβ1-4GlcNAcβ1-3[GalNAcα1-3(Fucα1-2)Galβ1-4(6S)GlcNAcβ1-6]GalNAc, and NeuAcα2-Galβ1-4GlcNAcβ1-2Manα1-3(Galβ1-4GlcNAcβ1-2Manα1-6)Manβ1-4GlcNAcβ1-4(Fucα1-6)GlcNAc.

In some embodiments, the GPC composition comprises, or alternatively consists essentially of, or yet further consists of between 20% and 30% Core 1 type oligosaccharides, between 30% and 50% (e.g., about 30, 35, 40, 45, or 50%) Core 2 type oligosaccharides, between 7% and 12% (e.g., about 7, 8, 9, 10, 11 or 12%) Core 3 type oligosaccharides, between 8% and 20% (e.g., about 8, 9, 10, 11, 12, 13, 14, 15, 16, 17, 18, 19, or 20%) Core 4 type oligosaccharides, between 2% and 6% (e.g., about 2, 3, 4, 5, or 6%) Core 5 type oligosaccharides and between 0.1% and 5% (e.g., about 0.1, 0.5, 0.8, 1, 1.5, 2, 2.5, 3, 3.5, 4, 4.5, or 5%) Core N type oligosaccharides as measured by liquid chromatograph-electrospray ionization tandem mass spectrometry (LC-ESI/MS) and further explained in Example 2.

The GPC composition can be combined with a carrier, such as a pharmaceutically acceptable carrier, that can optionally be combined with one or more of a preservative, a flavor agent, a cryoprotectant or a stabilizer. In one aspect, the composition is processed for the ease of storage, such as lyophilization.

### Treating or Preventing Tumors (Prophylaxys)

The present disclosure is based, at least in part, on the Applicant's surprising recognition that glycopeptide compositions are able to program T cells in a donor mammal to specifically attack and kill tumor cells, and these cells can surprisingly treat cancer when administered to a recipient mammal. Without being bound to any particular theory; it is believed that the presently described glycopeptide compositions comprise a glycome that mimics the altered glycome of the tumor microenvironment. The present composition is prepared by a process where the peptide backbone is cleaved without removing the attached glycan structures. It is also believed that the present compositions are effective at programming T cells to attack tumors because they comprise the right size of glycopeptides (less than or equal to 2 kDa, or between 8-14 amino acids) for presentation in MHCs. It is believed that effective presentation of the glycopeptides allows for T cell activation against the tumor glycome. Applicant has surprisingly observed that tumor cells that are programmed in a donor mammal by the compositions of the present disclosure display immunologic memory and can recognize and kill tumor cells when administered to a recipient mammal.

In one aspect, the present disclosure is directed to a method for treating cancer in a recipient mammal in need thereof comprising, or alternatively consisting essentially of, or yet further consisting of administering to the recipient mammal an effective amount of T cells isolated from a donor mammal, wherein the T cells were prepared by a method comprising, or alternatively consisting essentially of, or yet further consisting of administering to the donor mammal an effective amount of a composition comprising, or alternatively consisting essentially of, or yet further consisting of a plurality of glycopeptides, wherein at least 90%, at least 91%, at least 92%, at least 93%, at least 94%, at least 95%, at least 96%, at least 97%, at least 98%, at least 99%, or all of the plurality of glycopeptides in the composition are less than or equal to 2.2 KDa but greater than or equal to 0.5 KDa in size (e.g., 2.2, 2.1, 2, 1.9, 1.8, 1.7, 1.6, 1.5, 1.4, 1.3, 1.2, 1.1, 1, 0.9, 0.8, 0.7, 0.6, or 0.5 KDa, or any value therebetween) and wherein the plurality of glycopeptides comprises, or alternatively consists essentially of, or yet further consists of the following oligosaccharide structures: Fucα1-2Galβ1-3GalNAc, Galβ1-3(GlcNAcβ1-6)GalNAc, Galβ1-3(6SGlcNAcβ1-6)GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, Galβ1-3(Galβ1-4GlcNAcβ1-6)GalNAc, Fucα1-2Galβ1-3(6S-GlcNAcβ1-6)GalNAc, Galβ1-3(Galα1-3Galβ1-4GlcNAcβ1-6)GalNAc, and NeuAcα2-3Galβ1-3[(6S)GlcNAcβ1-6]GalNAc; thereby treating the cancer in the recipient mammal.

In another aspect, the present disclosure is directed to a method for treating cancer in a recipient mammal in need thereof comprising, or alternatively consisting essentially of, or yet further consisting of administering to the recipient mammal an effective amount of T cells isolated from a donor mammal, wherein the T cells were prepared by a method comprising, or alternatively consisting essentially of, or yet further consisting of administering to the donor mammal an effective amount of a composition comprising, or alternatively consisting essentially of, or yet further consisting of a plurality of glycopeptides, wherein at least 90%, at least 91%, at least 92%, at least 93%, at least 94%, at least 95%, at least 96%, at least 97%, at least 98%, at least 99%, or all of the plurality of glycopeptides in the composition comprise less than 15 amino acids greater than or equal to 8 amino acids (e.g., 14, 13, 12, 11, 10, 9 or 8 amino acids), and wherein the plurality of glycopeptides comprises, or alternatively consists essentially of, or yet further consists of the following oligosaccharide structures: Fucα1-2Galβ1-3GalNAc, Galβ1-3(GlcNAcβ1-6)GalNAc, Galβ1-3(6SGlcNAcβ1-6)GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, Galβ1-3(Galβ1-4GlcNAcβ1-6)GalNAc, Fucα1-2Galβ1-3(6S-GlcNAcβ1-6)GalNAc, Galβ1-3(Galα1-3Galβ1-4GlcNAcβ1-6)GalNAc, and NeuAcα2-3Galβ1-3[(6S)GlcNAcβ1-6]GalNAc; thereby treating the cancer in the recipient mammal.

In some embodiments, the T cells are autologous or allogeneic to the recipient mammal. In some embodiments, the donor mammal and the recipient mammal are the same (i.e., the T cells are autologous). In some embodiments, the donor mammal is different than the recipient mammal (i.e., the T cells are allogeneic).

In some embodiments, the T cells from a donor mammal administered with the glycopeptide compositions of the present disclosure are stored (e.g., cryopreserved) for future use. In some embodiments, the donor mammal is partially or fully Human Leukocyte Antigen (HLA) matched with the recipient mammal. In some embodiments, the donor mammal and the recipient mammal are matched in at least 6 HLAs.

In some embodiments, the T cells are HLA1-deficient T cells. In some embodiments, the T cells are HLA1-deficient T cells TCR-deficient T cells. In some embodiments, the T cells are genetically engineered to lack HLA1 (Human Leukocyte Antigen 1) and/or TCR (T cell receptor). In some embodiments, the T cells are universal T cells as described in Zhao, Juanjuan, et al. (Journal of hematology & oncology 11 (2018): 1-9), which is incorporated herein in its entirety.

In some embodiments, the composition is administered to the donor animal (e.g., a donor mammal) for 28 days or more (e.g., between 28 and 40 days) before isolating the T cells.

In some embodiments, the T cells isolated from the donor animal are cultured and expanded in vitro before administering the T cells to the recipient mammal.

In some embodiments, the plurality of glycopeptides is derived from porcine gastrointestinal mucins, and wherein the composition is obtained without subjecting the porcine gastrointestinal mucins to conditions or reagents that cause release of oligosaccharides from glycopeptides.

In some embodiments, the composition administered to the donor mammal comprises, or alternatively consists essentially of, or yet further consists of between about 0.5-1% (e.g., 0.5, 0.6, 0.7, 0.8, 0.9, 1% or any value therebetween) Fucα1-2Galβ1-3GalNAc containing glycopeptides by weight of the total composition. In some embodiments, the composition administered to the donor mammal comprises, or alternatively consists essentially of, or yet further consists of between about 0.5-1% (e.g., 0.5, 0.6, 0.7, 0.8, 0.9, 1% or any value therebetween) Galβ1-3(GlcNAcβ1-6)GalNAc containing glycopeptides by weight of the total composition. In some embodiments, the composition administered to the donor mammal comprises, or alternatively consists essentially of, or yet further consists of between about 3-5% (e.g., 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5 or any value therebetween) Galβ1-3(6SGlcNAcβ1-6)GalNAc containing glycopeptides by weight of the total composition. In some embodiments, the composition administered to the donor mammal comprises, or alternatively consists essentially of, or yet further consists of between about 1-3% (e.g., 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3 or any value therebetween) Galβ1-3(NeuAcα2-6)GalNAc by weight of the total composition. In some embodiments, the composition administered to the donor mammal comprises, or alternatively consists essentially of, or yet further consists of between about 1-3% % (e.g., 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3 or any value therebetween) NeuAcα2-3Galβ1-3GalNAc containing glycopeptides by weight of the total composition. In some embodiments, the composition administered to the donor mammal comprises, or alternatively consists essentially of, or yet further consists of between about 1-3% % (e.g., 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3 or any value therebetween) Galβ1-3(NeuGcα2-6)GalNAc containing glycopeptides by weight of the total composition. In some embodiments, the composition administered to the donor mammal comprises, or alternatively consists essentially of, or yet further consists of between about 1-3% (e.g., 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3 or any value therebetween) NeuGcα2-3Galβ1-3GalNAc containing glycopeptides by weight of the total composition. In some embodiments, the composition administered to the donor mammal comprises, or alternatively consists essentially of, or yet further consists of between about 0.5-1% (e.g., 0.5, 0.6, 0.7, 0.8, 0.9, 1% or any value therebetween) Galβ1-3(Galβ1-4GlcNAcβ1-6)GalNAc containing glycopeptides by weight of the total composition. In some embodiments, the composition administered to the donor mammal comprises, or alternatively consists essentially of, or yet further consists of between about 3-5% (e.g., 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5 or any value therebetween) Fucα1-2Galβ1-3(6S-GlcNAcβ1-6)GalNAc containing glycopeptides by weight of the total composition. In some embodiments, the composition administered to the donor mammal comprises, or alternatively consists essentially of, or yet further consists of between about 0.5-1% (e.g., 0.5, 0.6, 0.7, 0.8, 0.9, 1% or any value therebetween) Galβ1-3(Galα1-3Galβ1-4GlcNAcβ1-6)GalNAc containing glycopeptides by weight of the total composition. In some embodiments, the composition administered to the donor mammal comprises, or alternatively consists essentially of, or yet further consists of between about 0.5-1% (e.g., 0.5, 0.6, 0.7, 0.8, 0.9, 1% or any value therebetween) NeuAcα2-3Gal 1-3[(6S)GlcNAcβ1-6]GalNAc containing glycopeptides by weight of the total composition.

In some embodiments, the composition administered to the donor mammal comprises, or alternatively consists essentially of, or yet further consists of between about 0.5-1% Fucα1-2Galβ1-3GalNAc containing glycopeptides, between about 0.5-1% Galβ1-3(GlcNAcβ1-6)GalNAc containing glycopeptides, between about 3-5% Galβ1-3(6SGlcNAcβ1-6)GalNAc containing glycopeptides, between about 1-3% Galβ1-3(NeuAcα2-6)GalNAc, between about 1-3% NeuAcα2-3Galβ1-3GalNAc containing glycopeptides, between about 1-3% Galβ1-3(NeuGcα2-6)GalNAc containing glycopeptides, between about 1-3% NeuGcα2-3Galβ1-3GalNAc containing glycopeptides, between about 0.5-1% Galβ1-3(Galβ1-4GlcNAcβ1-6)GalNAc containing glycopeptides, between about 3-5% Fucα1-2Galβ1-3(6S-GlcNAcβ1-6)GalNAc containing glycopeptides, between about 0.5-1% Galβ1-3(Galα1-3Galβ1-4GlcNAcβ1-6)GalNAc containing glycopeptides, and between about 0.5-1% NeuAcα2-3Galβ1-3[(6S)GlcNAcβ1-6]GalNAc containing glycopeptides by weight of the total composition.

In some embodiments, the composition administered to the donor mammal comprises, or alternatively consists essentially of, or yet further consists of less than about 0.1%, 0.5%, 1%, 5%, 10%, 12%, 14%, 15%, 16%, 17%, 18%, 19%, 20%, 21%, 22%, 23%, or 24% free glycans (e.g., glycans not bond to peptides or proteins). In some embodiments, the composition has a free glycan content of less than 0.1% by weight. In some embodiments, the composition comprises substantially no free glycans.

In some embodiments, the composition administered to the donor mammal contains less than 5%, 4%, 3%, 2% o 1% of insoluble particles having a diameter greater than 7 μm. In some embodiments, the composition contains substantially no insoluble particles having a diameter greater than 7 μm. In some embodiments, the diameter of at least 95%, 96%, 97%, 98%, 99% or more of insoluble particles in the composition is less than or equal to 7 μm (e.g., 7, 6, 5, 4, 3, 2, 1 μm or smaller).

In some embodiments, the composition administered to the donor mammal comprises, or alternatively consists essentially of, or yet further consists of at least 1, 2, 3, 4, 5, 6, 7, 8, 9, 10, 11, 12, 13, 14, 15, 16, 20, 25, 30, or more sialylated glycopeptide-bound oligosaccharides (i.e., 1, 2, 3, etc. different sialylated glycopeptide-bound oligosaccharide structures). In some embodiments, the sialic acid content of the composition is greater than 5%, 10%, 15%, 20%, 22.5%, 25%, 27.5%, 30%, 32.5%, 35%, or 40% (w/w).

In some embodiments, the total glycoprotein content of the composition administered to the donor mammal is 12% or less (w/w). In some embodiments, the total glycoprotein content of the composition (w/w) is less than about 11%, 10%, 9%, 8%, 7%, 6%, 5%, 4%, 3%, 2%, 1%, 0.5%, or 0.1%. In some embodiments, the composition administered to the donor mammal comprises substantially no glycoproteins.

In some embodiments, the composition is administered to the donor mammal orally or rectally.

In some embodiments, the T cells from the donor mammal is administered as a monotherapy. In some embodiments, the T cells from the donor mammal is administered together with other cancer treatments, such as surgical resection, chemotherapy, immunotherapy (e.g., CAR-T, CAR-NK, or antibody-related therapies) or radiotherapy. In one aspect, the recipient mammal has been diagnosed with cancer, in one aspect, a cancer that is resistant or can become resistant to an immune checkpoint inhibitor. In a further aspect, the recipient mammal diagnosed with the cancer is treated. The recipient mammals not diagnosed with the cancer do not receive the composition.

The tumor is not limited and may be any kind of cancer, e.g., solid or blood cancer, e.g., carcinoma or sarcoma. In some embodiments, the cancer is ICI resistant. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

In some embodiments, the tumor is an immune checkpoint inhibitor (ICI)-resistant tumor. In some embodiments, the tumor is melanoma. In some embodiments, the tumor is ICI resistant melanoma. In some embodiments, the tumor is colorectal cancer (CRC). In some embodiments, the tumor is ICI resistant CRC. In some embodiments, the tumor is Stage I, Stage II, Stage III or Stage IV. In some embodiments, the tumor is localized or metastatic.

In some embodiments, the donor mammal and recipient mammal are canine (e.g., domesticated dog), feline (e.g., domesticated cat) or human.

In some embodiments, the cancer in the recipient mammal is refractory to immune checkpoint inhibitor therapy. In some embodiments, the immune checkpoint inhibitor is an anti-PD-1 antibody, an anti-CTLA-4 antibody, an anti-PD-L1 antibody, or an anti-PD-L2 antibody. In some embodiments, the immune checkpoint inhibitor is nivolumab, pembrolizumab, atezolizumab, durvalumab, pidilizumab, PDR001, BMS-936559, avelumab, or SHR-1210. In some embodiments, the immune checkpoint inhibitor is nivolumab.

In some embodiments, the method further comprises, or alternatively consists essentially of, or yet further consists of administering an immune checkpoint inhibitor (ICI) to the recipient mammal (e.g., human). The administration schedule for the T cells from the donor mammal and ICI are not limited. In some embodiments, the ICI is administered before, or before and simultaneously with, the T cells from the donor mammal. In some embodiments, the T cells from the donor mammal is administered starting at least 2 days, at least 3 days, at least 4 days, at least 5 days, at least 6 days, at least a week, at least 2 weeks, or at least a month before the ICI. In some embodiments, the T cells from the donor mammal is administered simultaneously with the ICI. In some embodiments, the T cells from the donor mammal is administered with the ICI or shortly after the ICI and administration is continued for at least 2 days, at least 3 days, at least 4 days, at least 5 days, at least 6 days, at least a week, at least 2 weeks, or at least a month. The immune checkpoint inhibitor (ICI) is not limited and may be any ICI described herein.

The composition administered to the donor mammal may be obtained from mucins from any suitable source. In some embodiments, the mucins are obtained from porcine gastrointestinal tract. In some embodiments, the mucins are obtained from porcine stomach. In some embodiments, the compositions are obtained by the methods provided in Examples 1-3 herein.

In some embodiments, the composition administered to the donor mammal is obtained from porcine gastric mucins, wherein: the composition is obtained without subjecting the mucins or a partially purified fraction thereof to conditions or reagents that cause substantial release of oligosaccharides from glycopeptides; the total oligosaccharide content of the composition is >10% (w/w); the ratio of glycopeptides:free glycans is >4:1 (w/w); and the total glycoprotein content of the composition is 12% or less (w/w).

In some embodiments, the total oligosaccharide content of the composition administered to the donor mammal is greater than about 10%, 12%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 50%, or 55% (w/w). As used herein, the total oligosaccharide content is the total weight of oligosaccharide in the composition. Such weight does not include the weight of proteins or peptides attached to the oligosaccharides. In some embodiments, the oligosaccharide content of the composition is greater than about 10% (w/w). In some embodiments, the oligosaccharide content of the composition is greater than or equal to about 15% (w/w). In some embodiments, the oligosaccharide content of the composition is greater than or equal to about 20% (w/w). In some embodiments, the oligosaccharide content comprises, or alternatively consists essentially of, or yet further consists of substantially all oligosaccharides bound to glycoprotein or glycopeptide without substantially any unbound oligosaccharides. Methods of determining oligosaccharide content are known in the art and are not limited. In some embodiments, oligosaccharide content is determined by HPAEC-PAD with an acid pre-treatment to hydrolyze the glycans into monosaccharides.

In some embodiments, the ratio of glycopeptides:free glycans in the composition administered to the donor mammal is >4:1 (w/w). In some embodiments, the ratio of glycopeptides:free glycans (w/w) is about 4:1, about 5:1, about 6:1, about 7:1, about 8:1, about 9:1, about 10:1, about 11:1, about 12:1, about 13:1, about 14:1, about 15:1, about 20:1, or more.

In some embodiments, the composition administered to the donor mammal comprises, or alternatively consists essentially of, or yet further consists of at least one glycopeptide-bound oligosaccharide having a general formulae selected from Hex1HexNAc1, HexNAc2, Hex1HexNAc1Sul1, HexNAc1deHex1Sul1, HexNAc2Sul1, NeuAc1HexNAc1, Hex2HexNAc1, Hex1HexNAc1deHex1, Hex1HexNAc2, HexNAc2deHex1, Hex1HexNAc1deHex1Sul1, Hex1HexNAcSul1, Hex2HexNAc1deHex1, NeuAc1Hex1HexNAc1, Hex1HexNAc1deHex2, Hex2HexNAc2, Hex1HexNAc2deHex1, Hex2HexNAc1deHex1Sul1, Hex1HexNAc1deHex2Sul1, Hex1HexNAc3, Hex1HexNAc2deHex1Sul1, NeuAc1Hex1HexNAc1deHex1, Hex2HexNAc2Sul1, NeuAc1Hex1HexNAc2, Hex1HexNAc3Sul1, Hex2HexNAc2deHex1, Hex1HexNAc2deHex2, Hex1HexNAc3deHex1, Hex2HexNAc3, Hex2HexNAc2deHex1Sul1, Hex1HexNAc4, Hex1HexNAc3deHex1Sul1, Hex3HexNAc2deHex1, NeuAc1Hex2HexNAc2, Hex2HexNAc2deHex2, Hex2HexNAc3deHex1, Hex3HexNAc2deHex1Sul1, Hex2HexNAc2deHex2Sul1, Hex2HexNAc4, Hex2HexNAc3deHex1Sul1, Hex3HexNAc3deHex1, Hex2HexNAc4deHex1, Hex3HexNAc3deHex1Sul1, and Hex4HexNAc3deHex1Sul1. In some embodiments, the composition comprises, or alternatively consists essentially of, or yet further consists of at least one or both of NeuAc1HexNAc1 and NeuAc1Hex1HexNAc1.

In some embodiments, the composition administered to the donor mammal comprises, or alternatively consists essentially of, or yet further consists of glycopeptide-bound oligosaccharides having at least 10, at least 20, at least 30, at least 40, at least 44, or all of the general formulae provided above (e.g., the composition comprises, or alternatively consists essentially of, or yet further consists of at least 10, 20, 30, 40, 44 glycopeptides, wherein each of the glycopeptides comprises, or alternatively consists essentially of, or yet further consists of a different glycopeptide-bound oligosaccharide from the above list). In some embodiments, the composition comprises, or alternatively consists essentially of, or yet further consists of glycopeptide-bound oligosaccharides having at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or all of the general formulae provided above.

In some embodiments, the composition administered to the donor mammal comprises, or alternatively consists essentially of, or yet further consists of at least one glycopeptide-bound oligosaccharide having a general formulae selected from Hex1HexNAc1, Hex1HexNAc1deHex1, Hex1HexNAc1deHex1Sul1, Hex1HexNAc1deHex2, Hex1HexNAc1deHex2Sul1, Hex1HexNAc1Sul1, Hex1HexNAc2, Hex1HexNAc2deHex1, Hex1HexNAc2deHex1Sul1, Hex1HexNAc2deHex2, Hex1HexNAc2Sul1, Hex1HexNAc3, Hex1HexNAc3deHex1, Hex1HexNAc3deHex1Sul1, Hex1HexNAc3Sul1, Hex1HexNAc4, Hex1HexNAcSul1, Hex2HexNAc1, Hex2HexNAc1deHex1, Hex2HexNAc1deHex1Sul1, Hex2HexNAc2, Hex2HexNAc2deHex1, Hex2HexNAc2deHex1Sul1, Hex2HexNAc2deHex2, Hex2HexNAc2deHex2Sul1, Hex2HexNAc2Sul1, Hex2HexNAc3, Hex2HexNAc3deHex1, Hex2HexNAc3deHex1Sul1, Hex2HexNAc4, Hex2HexNAc4deHex1, Hex3HexNAc2deHex1, Hex3HexNAc2deHex1Sul1, Hex3HexNAc3deHex1, Hex3HexNAc3deHex1Sul1, Hex4HexNAc3deHex1Sul1, HexNAc1deHex1Sul1, HexNAc2, HexNAc2deHex1, HexNAc2Sul1, NeuAc1Hex1HexNAc1, NeuAc1Hex1HexNAc1deHex1, NeuAc1Hex1HexNAc2, NeuAc1Hex2HexNAc2, and NeuAc1HexNAc1.

In some embodiments, the composition administered to the donor mammal comprises, or alternatively consists essentially of, or yet further consists of glycopeptide-bound oligosaccharides having at least 10, at least 20, at least, 30, at least 40, at least 45, or all (e.g., at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46) of the general formulae listed above. In some embodiments, the composition comprises, or alternatively consists essentially of, or yet further consists of at least 10 of the general formulae listed above, and each formula is present in the composition between 0.1% and 10% (e.g., 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%) of all glycopeptide-bound oligosaccharides in the composition.

In some embodiments, the composition administered to the donor mammal comprises, or alternatively consists essentially of, or yet further consists of at least one glycopeptide-bound oligosaccharide having a general formulae selected from Hex1HexNAc1, HexNAc2, Hex1HexNAc1Sul, Hex1NAc2Sul1, NeuAc1HexNAc1, NeuGc1HexNAc1, Hex1HexNAc1deHex1, Hex2HexNAc1, Hex1HexNAc2, HexNAc3, Hex1HexNAc2Sul1, NeuAc1Hex1HexNAc1, NeuGc1Hex1HexNAc1, Hex2HexNAc1deHex1, Hex1NAc3Sul1, NeuAc1HexNAc2, NeuGc1HexNAc2, Hex1HexNAc2deHex1, Hex2HexNAc2, Hex1HexNAc3, Hex1HexNAc2deHex1Sul1, NeuAc1Hex1HexNAc1deHex1, Hex2HexNAcSul1, NeuGc1Hex1HexNAc1deHex1, Hex1HexNAc3Sul1, NeuAc1Hex1HexNAc2, NeuGc1Hex1HexNAc2, Hex2HexNAc2deHex1, Hex3HexNAc2, Hex1HexNAc3deHex1, Hex2HexNAc3, NeuAc1Hex1HexNAc2Sul1, NeuAc2Hex1HexNAc1, NeuGc1Hex1HexNAc2Sul1, Hex2HexNAc2deHex1Sul1, NeuAc1NeuGc1Hex1HexNAc1, NeuGc2Hex1HexNAc1, Hex1HexNAc3deHex1Sul1, NeuAc1Hex1HexNAc2deHex1, Hex2HexNAc3Sul1, NeuGc1Hex1HexNAc2deHex1, NeuAc1Hex2HexNAc2, Hex2HexNAc2deHex2, NeuGc1Hex2HexNAc2, Hex2HexNAc3deHex1, NeuAc1Hex2HexNAc2Sul1, Hex2HexNAc2deHex2Sul1, NeuGc1Hex2HexNAc2Sul1, Hex1HexNAc4deHex1, NeuAc1Hex1HexNAc3Sul1, Hex1HexNAc3deHex2Sul1, NeuAc2Hex1HexNAc2, NeuGc1Hex1HexNAc3Sul1, Hex2HexNAc3deHex1Sul1, NeuAc1Hex2HexNAc2deHex1, NeuGc2Hex1HexNAc2, NeuGc1Hex2HexNAc2deHex1, NeuGc1Hex3HexNAc2, Hex1HexNAc4deHex1Sul1, NeuAc1Hex1HexNAc3deHex1, Hex2HexNAc3deHex2 NeuGc1Hex2HexNAc3, NeuAc1Hex2HexNAc2deHex1Sul1, Hex2HexNAc3deHex2Sul1, NeuAc1Hex2HexNAc3deHex1Sul1, NeuGc1Hex2HexNAc3deHex1Sul1, Hex2HexNAc4deHex2Sul1, Hex2HexNAc5deHex2Sul1, NeuAc1Hex5HexNAc4deHex1, and NeuAc1Hex4HexNAc4deHex2Sul1.

In some embodiments, the composition administered to the donor mammal comprises, or alternatively consists essentially of, or yet further consists of glycopeptide-bound oligosaccharides having at least 10, at least 20, at least, 30, at least 40, at least 45, at least 50, at least 55, at least 60, at least 65, (e.g., at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65) or all of the general formulae listed above. In some embodiments, the composition comprises, or alternatively consists essentially of, or yet further consists of at least 10 of the general formulae listed above, and each formula is present in the composition between 0.1% and 10% (e.g., 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%) of all glycopeptide-bound oligosaccharides in the composition. In some embodiments, the composition comprises, or alternatively consists essentially of, or yet further consists of at least one glycopeptide-bound oligosaccharide having a structure selected from Galβ1-3GalNAcol, GlcNAcβ1-4Galol, GlcNAcα1-4Galol, HexNAc-GlcAol, GlcNAcβ1-6GalNAcol, Galβ1-4(6S)GlcNAcol, 6SGlcNAc-Fucol, Galβ1-4(6S)GlcNAcol, (S)Galβ1-GlcNAcol, (S)Galβ1-GlcNAcol, 6SGlcNAcβ1-6GalNAcol, 6SGlcNAcβ1-3GalNAcol, NeuAc-HexNAcol, NeuAcα2-6GalNAcol, Galβ1-4GlcNAcβ1-3Gal minus H₂O, Fucα1-2(GalNAcβ1-3)Galol, Gal-GlcNAc-Fucol, Gal-GlcNAc-Fucol, Fucα1-2Galβ1-4GlcNAcol, Fucα1-2Galβ1-3GlcNAcol, Fucα1-2Galβ1-3GalNAcol, Galβ1-4GlcNAcβ1-3Gal, GlcNAcα1-4Galβ1-4GlcNAc minus H₂O, GlcNAc-GlcNAc-Fucol, Galβ1-3(GlcNAcβ1-6)GalNAcol, GlcNAcα1-4Galβ1-3GalNAcol, GlcNAcα1-4Galβ1-4GlcNAcol, Fucα1-2Galβ1-4(6S)GlcNAcol, Galβ1-4(Fucα1-3)(6S)GlcNAcol, 6SGalβ1-3(Fucα1-4)GlcNAcol, SGalβ1-3(GlcNAcβ1-6)GalNAcol, Galβ1-3(6SGlcNAcβ1-6)GalNAcol, Galβ1-4(6S)GlcNAcβ1-6GalNAcol, Fucα1-2Galβ-4GlcNAcβ1-3Gal minus H₂O, Galβ1-3(NeuAcα2-6)GalNAcol, NeuAcα2-3Galβ1-3GalNAcol, Fucα1-2Galβ1-4(Fucα1-3)GlcNAcol, Fucα1-2Galβ-4GlcNAcβ1-3Gal, GlcNAcα1-4Galβ1-4GlcNAcβ1-3Gal minus H₂O, Fucα1-2(GalNAcα1-3)Galβ1-4GlcNAcol, Fucα1-2(GalNAcβ1-3)Galβ1-3GalNAcol, GlcNAcα1-4Gal(Fuc)GlcNAcol, GlcNAcα1-4Gal(Fuc)GlcNAcol, Fucα1-2Galβ1-3(GlcNAcβ1-6)GalNAcol, GlcNAcα1-4Galβ1-4GlcNAcβ1-3Gal, Galβ1-3(Galβ1-4GlcNAcβ1-6)GalNAcol, GlcNAcα1-4Galβ1-4GlcNAcβ1-3Galol, Galβ1-3(Galβ1-4GlcNAcβ1-6)GalNAcol, Galβ1-4GlcNAcβ1-3Galβ1-3GalNAcol, Fucα1-2Galβ1-4(6S)GlcNAcβ1-4Gal minus H₂O, Fucα1-2(S)Galβ1-4GlcNAcβ1-4Gal minus H2O, Fucα1-2Gal(Fuc)(6S)GlcNAcol, Fucα1-2Galβ1-3(6S)GlcNAcβ1-4Galol, GlcNAcα1-4Galβ1-3(GlcNAcβ1-6)GalNAcol, GlcNAcα1-3Galβ1-4GlcNAcβ1-6GalNAcol, Fucα1-2(GalNAcα1-3)Galβ1-4(6S)GlcNAcol, Fucα1-2Galβ1-4(6S)GlcNAcβ1-3GalNAcol, Fucα1-2Galβ1-3(6S-GlcNAcβ1-6)GalNAcol, Fucα1-2Galβ1-3(NeuAcα2-6)GalNAcol, Galβ1-3[Galβ1-4(6S)GlcNAcβ1-6]GalNAcol, GlcNAcα1-4Galβ1-3(NeuAcα2-6)GalNAcol, GlcNAcα1-4Galβ1-3[(6S)GlcNAcβ1-6]GalNAcol, GalNAcα1-3(Fucα1-2)Galβ1-4GlcNAcβ1-3Gal minus H₂O, GlcNAcα1-4Galβ1-3(NeuAcα2-6)GalNAcol, Fucα1-2(GalNAcβ1-3)Gal-(Fuc)GlcNAcol, Galβ1-3(Fucα1-4)GlcNAcβ1-3Galβ1-3GalNAcol, Galβ1-4(Fucα1-3)GlcNAcβ1-3Galβ1-3GalNAcol, Fucα1-2Galβ1-4GlcNAcβ1-3Galβ1-4GlcNAcol, Galβ1-3(Fucα1-2Galβ1-4GlcNAcβ1-6)GalNAcol, Fucα1-2Galβ1-3(Galβ1-4GlcNAcβ1-6)GalNAcol, Fucα1-2(GalNAcα1-3)Galβ1-4GlcNAcβ1-6GalNAcol, Fucα1-2Galβ1-3(GalNAcβ1-4GlcNAcβ1-6)GalNAcol, Galβ1-4GlcNAcβ1-3(GlcNAcβ1-6)Galβ1-3GalNAcol, GlcNAcα1-4Galβ1-3(Galβ1-4GlcNAcβ1-6)GalNAcol, GalNAcα1-3(Fucα1-2)Galβ1-4(6S)GlcNAcβ1-3Gal minus H₂O, Fucα1-2Galβ1-4(6S)GlcNAcβ1-3Galβ1-3GalNAcol, Fucα1-2Galβ1-3[Galβ1-4(6S)GlcNAcβ1-6]GalNAcol, GlcNAcα1-4Galβ1-3(GalNAcβ1-4GlcNAcβ1-6)GalNAcol, GlcNAcβ1-3[Fucα1-2Galβ1-4(6S)GlcNAcβ1-6]GalNAcol, GalNAcα1-3(Fucα1-2)Galβ1-3(6SGlcNAcβ1-6)GalNAcol, Fucα1-2Galβ1-4GlcNAcβ1-3(Galβ1-4GlcNAcβ1-6)Gal, Fucα1-2Galβ1-4GlcNAcβ1-3(Galβ1-4GlcNAcβ1-6)Gal, Galβ1-4GlcNAcβ1-3Galβ1-3(NeuAcα2-6)GalNAcol, Fucα1-2Galβ1-3(Fucα1-2Galβ1-4GlcNAcβ1-6)GalNAcol, Galβ1-3[Fucα1-2(GalNAcα1-3)Galβ1-4GlcNAcβ1-6]GalNAcol, GlcNAcα1-4Galβ1-3(Fucα1-2Galβ1-4GlcNAcβ1-6)GalNAcol, Fucα1-2Galβ1-3(GlcNAcα1-4Galβ1-4GlcNAcβ1-6)GalNAcol, Fucα1-2Galβ1-4(6S)GlcNAcβ1-3(Galβ1-4GlcNAcβ1-6)Gal minus H₂O, Fucα1-2Galβ1-3[Fucα1-2Galβ1-4(6S)GlcNAcβ1-6]GalNAcol, GlcNAcα1-4Galβ1-3(GlcNAcα1-4Galβ1-4GlcNAcβ1-6)GalNAcol, Gal-GlcNAcβ1-3(GlcNAcα1-4Galβ1-4GlcNAcβ1-6)GalNAcol, GalNAcα1-3(Fucα1-2)Galβ1-4(6S)GlcNAcβ1-3Galβ1-3GalNAcol, Fucα1-2Galβ1-4GlcNAcβ1-3Galβ1-4GlcNAcβ1-3Galβ1-3GalNAcol, GlcNAcα1-4Galβ1-3[GalNAcα1-3(Fucα1-2)Galβ1-4GlcNAcβ1-6]GalNAcol, Fucα1-2Galβ1-4(6S)GlcNAcβ1-3Galβ1-4GlcNAcβ1-3Galβ1-4GlcNAcol, and Fucα1-2Galβ1-4(6S)GlcNAcβ1-3Galβ1-4GlcNAcβ1-3(Galβ1-4GlcNAcβ1-6)Gal minus H₂O. In some embodiments, the composition comprises, or alternatively consists essentially of, or yet further consists of Galβ1-3GalNAcol (T antigen) and NeuAcα2-3Galβ1-3GalNAcol (Sialyl T antigen). In some embodiments, the composition comprises, or alternatively consists essentially of, or yet further consists of Galβ1-3(Fucα1-4)GlcNAcβ1-3Galβ1-3GalNAcol (Lewis A) and Galβ1-4(Fucα1-3)GlcNAcβ1-3Galβ1-3GalNAcol (Lewis X).

In some embodiments, the administered to the donor mammal composition comprises, or alternatively consists essentially of, or yet further consists of glycopeptide-bound oligosaccharides having at least 10, at least 20, at least 50, at least 75, or all of the structures provided above (e.g., the composition comprises at least 10, 20, 50, 75, or all of the glycopeptides, wherein each of the glycopeptides comprises a different glycopeptide-bound oligosaccharide from the above list). In some embodiments, the composition comprises, or alternatively consists essentially of, or yet further consists of glycopeptide-bound oligosaccharides having at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or all of the structures provided above.

In some embodiments, the composition comprises, or alternatively consists essentially of, or yet further consists of glycopeptide-bound oligosaccharides having at least 10, at least 20, at least 50, at least 75, or all of the structures provided above (e.g., the composition comprises at least 10, 20, 50, 75, or 99 glycopeptides, wherein each of the glycopeptides comprises, or alternatively consists essentially of, or yet further consists of a different glycopeptide-bound oligosaccharide from the above list).

In some embodiments, the composition has a salt content of less than between about 5% and 1%. In some embodiments, the composition has a salt content of less than about 2%. In some embodiments, the composition has a salt content of less than about 1%.

In some embodiments, the composition has a pH of less than 7.5 (e.g., pH 7.4, 7.3, 7.2, 7.1, 7, 6.9, 6.8, 6.7, 6.6, 6.5, 6.4, 6.3, 6.2, 6.1, or 6).

In some embodiments, the composition does not comprise more than about 5%, more than about 4%, more than about 3%, more than about 2%, more than about 1%, more than about 0.5%, or more than about 0.1% free glycans by weight. In some embodiments, the composition has a free glycan content of less than 1% by weight. In some embodiments, the composition has a free glycan content of less than 0.1% by weight. In some embodiments, the composition has a free glycan content of substantially zero. The phrase “free glycans” refers to glycans that are not attached to a protein or polypeptide.

In some embodiments, the composition comprising, or alternatively consisting essentially of, or yet further consisting of a mixture of glycopeptides is obtained from porcine intestinal mucins or a partially purified fraction thereof, wherein: the composition is obtained without subjecting the mucins or the partially purified fraction thereof to conditions or reagents that release oligosaccharides from glycopeptides; the composition has a total glycan content of greater than 10%; the composition comprises, or alternatively consists essentially of, or yet further consists of glycopeptide-bound oligosaccharides having each of the following general formulae: Hex1HexNAc1, HexNAc2, NeuAc1HexNAc1, NeuGc1HexNAc1, Hex1HexNAc1Fuc1, Hex1HexNAc2, Hex1HexNAc2Sul1, NeuAc1Hex1HexNAc1, NeuGc1Hex1HexNAc1, NeuAc1HexNAc2, NeuGc1HexNAc2, Hex1HexNAc2Fuc1, Hex1HexNAc2Fuc1Sul1, NeuAc1Hex1HexNAc1Fuc1, Hex1HexNAc3Sul1, Hex2HexNAc2Fuc1, Hex1HexNAc3Fuc1Sul1, Hex2HexNAc2Fuc2Sul1, and the composition does not substantially contain insoluble particles having a diameter greater than 7 μm or compounds having a molecular weight of greater than about 3 kDa.

In some embodiments, the composition comprises, or alternatively consists essentially of, or yet further consists of glycopeptide-bound oligosaccharides having each of the following general formulae: Hex2HexNAc3deHex1Sul2, Hex2HexNAc3deHex1Sul2, NeuGc1Hex2HexNAc3, NeuAc1Hex2HexNAc2deHex1Sul1, Hex2HexNAc4Sul2, Hex2HexNAc4Sul2, NeuAc1Hex2HexNAc3Sul1, Hex2HexNAc3deHex2Sul1, Hex2HexNAc3deHex2Sul1, Hex2HexNAc3deHex2Sul1, Hex2HexNAc3deHex2Sul1, Hex2HexNAc3deHex1Sul3, NeuGc1Hex2HexNAc3Sul1, NeuGc1Hex2HexNAc3Sul1, Hex2HexNAc3deHex2Sul2, Hex2HexNAc3deHex2Sul2, Hex2HexNAc3deHex2Sul2, Hex2HexNAc3deHex2Sul2, NeuGc1Hex2HexNAc3deHex1, NeuGc1Hex2HexNAc3Sul2, Hex3HexNAc3deHex1Sul2, NeuGc1Hex2HexNAc2deHex2Sul1, Hex2HexNAc4deHex1Sul2, NeuAc1Hex2HexNAc3deHex1Sul1, Hex2HexNAc3deHex3Sul1, NeuGc1Hex2HexNAc3deHex1Sul1, NeuGc1Hex2HexNAc3deHex1Sul1, Hex3HexNAc3deHex2Sul1, NeuGc1Hex2HexNAc2deHex2Sul2, NeuGc1Hex3HexNAc2deHex1Sul2, Hex2HexNAc4deHex2Sul1, Hex2HexNAc4deHex2Sul1, Hex2HexNAc4deHex2Sul1, NeuGc1Hex2HexNAc4Sul1, Hex3Hex4deHex1Sul1, Hex2HexNAc3deHex3Sul2, Hex2HexNAc3deHex3Sul2, NeuGc1Hex2HexNAc3deHex1Sul2, NeuGc1Hex2HexNAc3deHex1Sul2, NeuGc1Hex2HexNAc3deHex1Sul2, NeuGc1Hex3HexNAc2deHex2Sul1, NeuAc1Hex2HexNAc4deHex1, Hex2HexNAc4deHex2Sul2, Hex2HexNAc4deHex2Sul2, NeuGc1Hex2HexNAc4deHex1, Hex2HexNAc3deHex4Sul1, Hex3HexNAc4deHex1Sul2, NeuGc1Hex2HexNAc3deHex2Sul1, NeuGc1Hex2HexNAc3deHex2Sul1, NeuGc1Hex3HexNAc3deHex1Sul1, NeuGc1Hex2HexNAc2deHex3Sul2, Hex2HexNAc4deHex3Sul1, NeuGc1Hex2HexNAc4deHex1Sul1, NeuGc1Hex2HexNAc4deHex1Sul1, NeuGc1Hex2HexNAc4deHex1Sul1, NeuGc1Hex2HexNAc4deHex1Sul1, NeuGc1Hex2HexNAc3deHex2Sul2, Hex3HexNAc3deHex3Sul2, NeuGc1Hex3HexNAc3deHex1Sul2, Hex2HexNAc5deHex2Sul1, Hex2HexNAc4deHex3Sul2, NeuGc1Hex2HexNAc4deHex2Sul1, NeuGc1Hex2HexNAc4deHex2Sul1, NeuGc1Hex3HexNAc4deHex1Sul1, Hex2HexNAc5deHex3Sul1, NeuGc1Hex2HexNAc4deHex2Sul2, NeuGc1Hex2HexNAc4deHex2Sul2, NeuGc1Hex2HexNAc4deHex2Sul2, NeuGc1Hex3HexNAc4deHex1Sul2, Hex3HexNAc4deHex4Sul2, NeuAc1Hex5HexNAc4deHex1, NeuAc1Hex4HexNAc4deHex2Sul1, NeuGc1Hex3HexNAc4deHex4Sul1, NeuGc1Hex4HexNAc4deHex3Sul1, and NeuGc1Hex3HexNAc4deHex4Sul2. In some embodiments, the composition comprises, or alternatively consists essentially of, or yet further consists of at least one, two or all three of NeuAc1HexNAc1, NeuAc2Hex1HexNAc1, and NeuAc1Hex1HexNAc1.

In some embodiments, the sialic acid content of the composition is greater than 5%, 10%, 15%, 20%, 22.5%, 25%, 27.5%, 30%, 32.5%, 35%, or 40% (w/w). In some embodiments, the sialic acid content of the composition is greater than 25% (w/w) and less than 50% (e.g., 25, 30, 35, 40, 45, or 49%). In some embodiments, sialic acid content is determined by LC-MS/MS.

In some embodiments, the composition comprises, or alternatively consists essentially of, or yet further consists of glycopeptide-bound oligosaccharides having at least 7, 14, 21 or all 28 of the following structures: Galβ1-3GalNAc, GlcNAcβ1-6GalNAc, NeuAcα2-6GalNAc, NeuGcα2-6GalNAc, Fucα1-2Galβ1-3GalNAc, Gal+GlcNAcβ1-6GalNAc, Galβ1-3(GlcNAcβ1-6)GalNAc, Galβ1-3GlcNAcβ1-6GalNAc, Galβ1-3(GlcNAcβ1-6)GalNAc, Galβ1-3(6SGlcNAcβ1-6)GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, GlcNAc-(NeuAcα2-6)GalNAc, GalNAc-(NeuAcα2-6)GalNAc, HexNAc-(NeuGcα2-6)GalNAc, Fucα1-2(GalNAcβ1-3)Galβ1-3GalNAc, Fucα1-2Galβ1-4GlcNAcβ1-6GalNAc, Fucα1-2Galβ1-3(GlcNAcβ1-6)GalNAc, Fucα1-2Galβ1-3(6S-GlcNAcβ1-6)GalNAc, Fucα1-2Galβ1-3(NeuAcβ2-6)GalNAc, GlcNAcβ1-3[Galβ1-4(6S)GlcNAcβ1-6]GalNAc, Galβ1-4GlcNAcβ1-3[(6S)GlcNAcβ1-6]GalNAc, Galβ1-3(Fucα1-2Galβ1-4GlcNAcβ1-6)GalNAc, Fucα1-2Galβ1-4(6S)GlcNAcβ1-6[GlcNAcβ1-3]GalNAc, GlcNAcβ1-3[Fucα1-2Galβ1-3(6S-)GlcNAcβ1-6]GalNAc, and Fucα1-2Galβ1-3[Fucα1-2Galβ1-4(6S)GlcNAcβ1-6]GalNAc.

In some embodiments, the composition comprises, or alternatively consists essentially of, or yet further consists of at least 1, 2, 3, 4, 5, 6, 7, 8, 9, or all 10 sialylated glycopeptide-bound oligosaccharide selected from the following: NeuAcα2-6GalNAc, NeuGcα2-6GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, GlcNAc-(NeuAcα2-6)GalNAc, GalNAc-(NeuAcα2-6)GalNAc, HexNAc-(NeuGcα2-6)GalNAc, and Fucα1-2Galβ1-3(NeuAcβ2-6)GalNAc. In some embodiments, the composition comprises, or alternatively consists essentially of, or yet further consists of NeuAcα2-3Galβ1-3GalNAc (Sialyl-T antigen), NeuAcα2-6GalNAc (Sialyl Tn antigen), and NeuAcα2-3Galβ1-3(NeuAcα2-6)GalNAc (Disialyl T antigen).

Another aspect of the disclosure is directed to a method for treating cancer in a subject who has been diagnosed with cancer comprising, or alternatively consisting essentially of, or yet further consisting of administering to the subject an effective amount of T cells isolated from a donor mammal, wherein the T cells were prepared by a method comprising, or alternatively consisting essentially of, or yet further consisting of administering to the donor mammal an effective amount of a composition comprising, or alternatively consisting essentially of, or yet further consisting of glycopeptides produced by a method comprising, or alternatively consisting essentially of, or yet further consisting of: (a) dissolving gastrointestinal mucin in water comprising, or alternatively consisting essentially of, or yet further consisting of calcium hydroxide; (b) adding diatomaceous earth to (a) and filtering the resulting solution; (c) adding a cationic substance to (b); and (d) filtering and concentrating the solution from (c), thereby producing the composition.

In some embodiments, step (g) removes particles bigger than 2 μm are removed from the composition.

In some embodiments, the method further comprises adjusting the pH of the solution in (b) using carbon dioxide.

In some embodiments, the dissolving is achieved at about 60° C.

In some embodiments, the cationic substance comprises, or alternatively consists essentially of, or yet further consists of an ion exchange hydrogen form resin.

Another aspect of the disclosure is directed to a method for treating cancer in a subject who has been diagnosed with cancer comprising, or alternatively consisting essentially of, or yet further consisting of administering to the subject an effective amount of T cells isolated from a donor mammal, wherein the T cells were prepared by a method comprising, or alternatively consisting essentially of, or yet further consisting of administering to the donor mammal an effective amount of a composition comprising, or alternatively consisting essentially of, or yet further consisting of glycopeptides produced by a method comprising, or alternatively consisting essentially of, or yet further consisting of: (a) stabilizing gastrointestinal mucin at pH 5.0; (b) desalinating the stabilized mucin using dialysis; (c) concentrating the desalinated mucin; (d) subjecting the concentrate from (d) to diafiltration; thereby producing the composition. In one aspect, the subject has been diagnosed a cancer that is resistant or can become resistant to an immune checkpoint inhibitor. In a further aspect, the subject diagnosed with the cancer is treated. Subjects not diagnosed with the cancer do not receive the composition.

In some embodiments, the concentrating in (c) is achieved by evaporation with a rotary evaporator at least 80° C.

In some embodiments, the diafiltration is performed at a 2 micron (μm) cut off, where particles bigger than 2 μm are removed from the composition.

In some embodiments, the effective amount of the composition is about 0.2-0.8 grams per kilogram of the subject.

In some embodiments, the method further comprises, or alternatively consists essentially of, or yet further consists of administering a chemotherapeutic agent before, after, or simultaneously with the T cells isolated from the donor mammal. In some embodiments, the chemotherapeutic agent is an immune checkpoint inhibitor.

In some embodiments, the composition administered to the donor mammal has substantially no free glycans. In some embodiments, the composition administered to the donor mammal has a pH of less than 7.5 (e.g., pH 7.4, 7.3, 7.2, 7.1, 7, 6.9, 6.8, 6.7, 6.6, 6.5, 6.4, 6.3, 6.2, 6.1, or 6).

In some embodiments, the composition administered to the donor mammal is a powder. In some embodiments, the composition administered to the donor mammal further comprises, or alternatively consists essentially of, or yet further consists of one or more excipients or carriers. In some embodiments, the glycopeptide composition administered to the donor mammal is administered orally or rectally.

In some embodiments, the composition administered to the donor mammal may take the form of a slurry, powder, or liquid. In some embodiments, the composition administered to the donor mammal is a powder.

In some embodiments of the methods disclosed herein, the composition administered to the donor mammal comprises, or alternatively consists essentially of, or yet further consists of at least one, at least two, at least three, at least four, at least five, or all six glycopeptide-bound oligosaccharide having a structure selected from Galβ1-3GalNAcol (T antigen), NeuAcα2-6GalNAcol (Sialyl Tn antigen), Galβ1-4(Fucα1-3)(6S)GlcNAcol (Lewis X), 6SGalβ1-3(Fucα1-4)GlcNAcol (Lewis A), NeuAcα2-3Galol-3(NeuAcα2-6)GalNAc (Disialyl T antigen), and NeuAcα2-3Galβ1-3GalNAcol (Sialyl T antigen).

Routes of administering the composition are not limited and may be any suitable route. In some embodiments, the composition is administered orally, parenteral, or rectally.

In some embodiments, the composition administered to the donor mammal is in the form of a pharmaceutical composition. In some embodiments, the composition further comprises one or more excipients or carriers. In some embodiments, the composition comprises a pharmaceutically-acceptable carrier. The term “pharmaceutically-acceptable carrier”, as used herein, means one or more compatible solid or liquid vehicles, fillers, diluents, or encapsulating substances which are suitable for administration to a human or non-human animal. In some embodiments, a pharmaceutically-acceptable carrier is a non-toxic material that does not interfere with the effectiveness of the biological activity of the active ingredients (i.e., the glycopeptides). The term “compatible”, as used herein, means that the components of the pharmaceutical compositions are capable of being comingled with an agent (i.e., the glycopeptides), and with each other, in a manner such that there is no interaction which would substantially reduce the pharmaceutical efficacy of the pharmaceutical composition under ordinary use situations. Pharmaceutically-acceptable carriers should be of sufficiently high purity and sufficiently low toxicity to render them suitable for administration to the human or non-human animal being treated.

Some examples of substances which can serve as pharmaceutically-acceptable carriers are pyrogen-free water; isotonic saline; phosphate buffer solutions; sugars such as lactose, glucose, and sucrose; starches such as corn starch and potato starch; cellulose and its derivatives, such as sodium carboxymethylcellulose, ethylcellulose, cellulose acetate; powdered tragacanth; malt; gelatin; talc; stearic acid; magnesium stearate; calcium sulfate; vegetable oils such as peanut oil, cottonseed oil, sesame oil, olive oil, corn oil and oil of theobrama; polyols such as propylene glycol, glycerin, sorbitol, mannitol, and polyethylene glycol; sugar; alginic acid; cocoa butter (suppository base); emulsifiers, such as the Tweens; as well as other non-toxic compatible substances used in pharmaceutical formulation. Wetting agents and lubricants such as sodium lauryl sulfate, as well as coloring agents, flavoring agents, excipients, tableting agents, stabilizers, antioxidants, and preservatives, can also be present. It will be appreciated that a pharmaceutical composition can contain multiple different pharmaceutically acceptable carriers.

In some embodiments, the composition is administered to the donor mammal for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days. In some embodiments, the composition is administered to the donor mammal daily for 28 days or more.

In some embodiments, the composition is administered to a donor mammal orally or via suppository in an effective amount of about 10-80 grams per day. In some embodiments, the composition is administered to a donor mammal orally or via suppository in an effective amount of about 0.2 to 0.8 (e.g., 0.2, 0.3, 0.4, 0.5, 0.6, 0.7, or 0.8) grams per kg of the subject's weight. In some embodiments, the composition is administered to a donor mammal orally or via suppository in an effective amount of about 0.4 to 0.6 (e.g., 0.4, 0.5, or 0.6) grams per kg of the subject's weight. In some embodiments, the composition is administered to a donor mammal orally or via suppository in an effective amount of about 0.5 to 0.6 (0.5 or 0.6) grams per kg of the subject's weight. In some embodiments, the amount per day is divided into 1, 2, 3, 4, 5, or 6 doses per day.

In some embodiments, the recipient mammal has a tumor that is refractory to or has developed resistance to an immune checkpoint inhibitor therapy.

In some embodiments, the recipient mammal has stage 3 or 4 melanoma, CRC, or metastatic CRC. In some embodiments, the recipient mammal has breast cancer or lung cancer.

In some embodiments, administration of the composition to a donor mammal results in an increased level or activity of T cells cross-reactive to a glycan tumor antigen and a glycan present on a glycopeptide of the composition in the T cells of the donor mammal. In some embodiments, the T cells are CD8+ T cells. In some embodiments, the T cells are CD4+ T cells. In some embodiments, the composition is administered orally or via a suppository.

### Compositions Comprising Reprogrammed T Cells

Another aspect of the disclosure is directed to a composition comprising, or alternatively consisting essentially of, or yet further consisting of isolated T cells and a cryoprotective agent, wherein the T cells were prepared by a method comprising, or alternatively consisting essentially of, or yet further consisting of administering to a donor mammal an effective amount of a composition comprising, or alternatively consisting essentially of, or yet further consisting of a plurality of glycopeptides, wherein at least 90%, at least 91%, at least 92%, at least 93%, at least 94%, at least 95%, at least 96%, at least 97%, at least 98%, at least 99%, or all of the plurality of glycopeptides in the composition are less than or equal to 2.2 KDa but greater than or equal to 0.5 KDa in size (e.g., 2.2, 2.1, 2, 1.9, 1.8, 1.7, 1.6, 1.5, 1.4, 1.3, 1.2, 1.1, 1, 0.9, 0.8, 0.7, 0.6, or 0.5 KDa, or any value therebetween) and wherein the plurality of glycopeptides comprises, or alternatively consists essentially of, or yet further consists of the following oligosaccharide structures: Fucα1-2Galβ1-3GalNAc, Galβ1-3(GlcNAcβ1-6)GalNAc, Galβ1-3(6SGlcNAcβ1-6)GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, Galβ1-3(Galβ1-4GlcNAcβ1-6)GalNAc, Fucα1-2Galβ1-3(6S-GlcNAcβ1-6)GalNAc, Galβ1-3(Galα1-3Galβ1-4GlcNAcβ1-6)GalNAc, and NeuAcα2-3Galβ1-3[(6S)GlcNAcβ1-6]GalNAc; thereby treating the cancer in the recipient mammal.

Another aspect of the disclosure is directed to a composition comprising, or alternatively consisting essentially of, or yet further consisting of isolated T cells and a cryoprotective agent, wherein the T cells were prepared by a method comprising, or alternatively consisting essentially of, or yet further consisting of administering to a donor mammal an effective amount of a composition comprising, or alternatively consisting essentially of, or yet further consisting of a plurality of glycopeptides, wherein at least 90%, at least 91%, at least 92%, at least 93%, at least 94%, at least 95%, at least 96%, at least 97%, at least 98%, at least 99%, or all of the plurality of glycopeptides in the composition comprise less than 15 amino acids greater than or equal to 8 amino acids (e.g., 14, 13, 12, 11, 10, 9 or 8 amino acids), and wherein the plurality of glycopeptides comprises, or alternatively consists essentially of, or yet further consists of the following oligosaccharide structures: Fucα1-2Galβ1-3GalNAc, Galβ1-3(GlcNAcβ1-6)GalNAc, Galβ1-3(6SGlcNAcβ1-6)GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, Galβ1-3(Galβ1-4GlcNAcβ1-6)GalNAc, Fucα1-2Galβ1-3(6S-GlcNAcβ1-6)GalNAc, Galβ1-3(Galα1-3Galβ1-4GlcNAcβ1-6)GalNAc, and NeuAcα2-3Galβ1-3[(6S)GlcNAcβ1-6]GalNAc; thereby treating the cancer in the recipient mammal.

In some embodiments, the T cells are HLA1-deficient T cells. In some embodiments, the T cells are HLA1-deficient T cells TCR-deficient T cells. In some embodiments, the T cells are genetically engineered to lack HLA1 (Human Leukocyte Antigen 1) and/or TCR (T cell receptor). In some embodiments, the T cells are universal T cells as described in Zhao, Juanjuan, et al. (Journal of hematology & oncology 11 (2018): 1-9), which is incorporated herein in its entirety.

In some embodiments, the composition is administered to the donor mammal for 28 days or more (e.g., between 28 and 40 days) before isolating the T cells.

In some embodiments, the T cells isolated from the donor animal are cultured and expanded in vitro before administering the T cells to the recipient mammal.

In some embodiments, the plurality of glycopeptides is derived from porcine gastrointestinal mucins, and wherein the composition is obtained without subjecting the porcine gastrointestinal mucins to conditions or reagents that cause release of oligosaccharides from glycopeptides.

In some embodiments, the composition administered to the donor mammal comprises, or alternatively consists essentially of, or yet further consists of between about 0.5-1% (e.g., 0.5, 0.6, 0.7, 0.8, 0.9, 1% or any value therebetween) Fucα1-2Galβ1-3GalNAc containing glycopeptides by weight of the total composition. In some embodiments, the composition administered to the donor mammal comprises, or alternatively consists essentially of, or yet further consists of between about 0.5-1% (e.g., 0.5, 0.6, 0.7, 0.8, 0.9, 1% or any value therebetween) Galβ1-3(GlcNAcβ1-6)GalNAc containing glycopeptides by weight of the total composition. In some embodiments, the composition administered to the donor mammal comprises, or alternatively consists essentially of, or yet further consists of between about 3-5% (e.g., 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5 or any value therebetween) Galβ1-3(6SGlcNAcβ1-6)GalNAc containing glycopeptides by weight of the total composition. In some embodiments, the composition administered to the donor mammal comprises, or alternatively consists essentially of, or yet further consists of between about 1-3% (e.g., 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3 or any value therebetween) Galβ1-3(NeuAcα2-6)GalNAc by weight of the total composition. In some embodiments, the composition administered to the donor mammal comprises, or alternatively consists essentially of, or yet further consists of between about 1-3% % (e.g., 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3 or any value therebetween) NeuAcα2-3Galβ1-3GalNAc containing glycopeptides by weight of the total composition. In some embodiments, the composition administered to the donor mammal comprises, or alternatively consists essentially of, or yet further consists of between about 1-3% % (e.g., 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3 or any value therebetween) Galβ1-3(NeuGcα2-6)GalNAc containing glycopeptides by weight of the total composition. In some embodiments, the composition administered to the donor mammal comprises, or alternatively consists essentially of, or yet further consists of between about 1-3% (e.g., 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3 or any value therebetween) NeuGcα2-3Galβ1-3GalNAc containing glycopeptides by weight of the total composition. In some embodiments, the composition administered to the donor mammal comprises, or alternatively consists essentially of, or yet further consists of between about 0.5-1% (e.g., 0.5, 0.6, 0.7, 0.8, 0.9, 1% or any value therebetween) Galβ1-3(Galβ1-4GlcNAcβ1-6)GalNAc containing glycopeptides by weight of the total composition. In some embodiments, the composition administered to the donor mammal comprises, or alternatively consists essentially of, or yet further consists of between about 3-5% (e.g., 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5 or any value therebetween) Fucα1-2Galβ1-3(6S-GlcNAcβ1-6)GalNAc containing glycopeptides by weight of the total composition. In some embodiments, the composition administered to the donor mammal comprises, or alternatively consists essentially of, or yet further consists of between about 0.5-1% (e.g., 0.5, 0.6, 0.7, 0.8, 0.9, 1% or any value therebetween) Galβ1-3(Galα1-3Galβ1-4GlcNAcβ1-6)GalNAc containing glycopeptides by weight of the total composition. In some embodiments, the composition administered to the donor mammal comprises, or alternatively consists essentially of, or yet further consists of between about 0.5-1% (e.g., 0.5, 0.6, 0.7, 0.8, 0.9, 1% or any value therebetween) NeuAcα2-3Galβ1-3[(6S)GlcNAcβ1-6]GalNAc containing glycopeptides by weight of the total composition.

In some embodiments, the composition administered to the donor mammal comprises, or alternatively consists essentially of, or yet further consists of between about 0.5-1% Fucα1-2Galβ1-3GalNAc containing glycopeptides, between about 0.5-1% Galβ1-3(GlcNAcβ1-6)GalNAc containing glycopeptides, between about 3-5% Galβ1-3(6SGlcNAcβ1-6)GalNAc containing glycopeptides, between about 1-3% Galβ1-3(NeuAcα2-6)GalNAc, between about 1-3% NeuAcα2-3Galβ1-3GalNAc containing glycopeptides, between about 1-3% Galβ1-3(NeuGcα2-6)GalNAc containing glycopeptides, between about 1-3% NeuGcα2-3Galol-3GalNAc containing glycopeptides, between about 0.5-1% Galβ1-3(Galβ1-4GlcNAcβ1-6)GalNAc containing glycopeptides, between about 3-5% Fucα1-2Galβ1-3(6S-GlcNAcβ1-6)GalNAc containing glycopeptides, between about 0.5-1% Galβ1-3(Galα1-3Galβ1-4GlcNAcβ1-6)GalNAc containing glycopeptides, and between about 0.5-1% NeuAcα2-3Galβ1-3[(6S)GlcNAcβ1-6]GalNAc containing glycopeptides by weight of the total composition.

In some embodiments, the composition administered to the donor mammal comprises, or alternatively consists essentially of, or yet further consists of less than about 0.1%, 0.5%, 1%, 5%, 10%, 12%, 14%, 15%, 16%, 17%, 18%, 19%, 20%, 21%, 22%, 23%, or 24% free glycans (e.g., glycans not bond to peptides or proteins). In some embodiments, the composition has a free glycan content of less than 0.1% by weight. In some embodiments, the composition comprises substantially no free glycans.

In some embodiments, the composition administered to the donor mammal contains less than 5%, 4%, 3%, 2% o 1% of insoluble particles having a diameter greater than 7 μm. In some embodiments, the composition contains substantially no insoluble particles having a diameter greater than 7 μm. In some embodiments, the diameter of at least 95%, 96%, 97%, 98%, 99% or more of insoluble particles in the composition is less than or equal to 7 μm (e.g., 7, 6, 5, 4, 3, 2, 1 μm or smaller).

In some embodiments, the composition administered to the donor mammal comprises, or alternatively consists essentially of, or yet further consists of at least 1, 2, 3, 4, 5, 6, 7, 8, 9, 10, 11, 12, 13, 14, 15, 16, 20, 25, 30, or more sialylated glycopeptide-bound oligosaccharides (i.e., 1, 2, 3, etc. different sialylated glycopeptide-bound oligosaccharide structures). In some embodiments, the sialic acid content of the composition is greater than 5%, 10%, 15%, 20%, 22.5%, 25%, 27.5%, 30%, 32.5%, 35%, or 40% (w/w).

In some embodiments, the total glycoprotein content of the composition administered to the donor mammal is 12% or less (w/w). In some embodiments, the total glycoprotein content of the composition (w/w) is less than about 11%, 10%, 9%, 8%, 7%, 6%, 5%, 4%, 3%, 2%, 1%, 0.5%, or 0.1%. In some embodiments, the composition administered to the donor mammal comprises substantially no glycoproteins.

In some embodiments, the composition is administered to the donor mammal orally or rectally.

The composition administered to the donor mammal may be obtained from mucins from any suitable source. In some embodiments, the mucins are obtained from porcine gastrointestinal tract. In some embodiments, the mucins are obtained from porcine stomach. In some embodiments, the compositions are obtained by the methods provided in Examples 1-3 herein.

In some embodiments, the composition administered to the donor mammal is obtained from porcine gastric mucins, wherein: the composition is obtained without subjecting the mucins or a partially purified fraction thereof to conditions or reagents that cause substantial release of oligosaccharides from glycopeptides; the total oligosaccharide content of the composition is >10% (w/w); the ratio of glycopeptides:free glycans is >4:1 (w/w); and the total glycoprotein content of the composition is 12% or less (w/w).

In some embodiments, the total oligosaccharide content of the composition administered to the donor mammal is greater than about 10%, 12%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 50%, or 55% (w/w). As used herein, the total oligosaccharide content is the total weight of oligosaccharide in the composition. Such weight does not include the weight of proteins or peptides attached to the oligosaccharides. In some embodiments, the oligosaccharide content of the composition is greater than about 10% (w/w). In some embodiments, the oligosaccharide content of the composition is greater than or equal to about 15% (w/w). In some embodiments, the oligosaccharide content of the composition is greater than or equal to about 20% (w/w). In some embodiments, the oligosaccharide content comprises, or alternatively consists essentially of, or yet further consists of substantially all oligosaccharides bound to glycoprotein or glycopeptide without substantially any unbound oligosaccharides. Methods of determining oligosaccharide content are known in the art and are not limited. In some embodiments, oligosaccharide content is determined by HPAEC-PAD with an acid pre-treatment to hydrolyze the glycans into monosaccharides.

In some embodiments, the ratio of glycopeptides:free glycans in the composition administered to the donor mammal is >4:1 (w/w). In some embodiments, the ratio of glycopeptides:free glycans (w/w) is about 4:1, about 5:1, about 6:1, about 7:1, about 8:1, about 9:1, about 10:1, about 11:1, about 12:1, about 13:1, about 14:1, about 15:1, about 20:1, or more.

In some embodiments, the composition administered to the donor mammal comprises, or alternatively consists essentially of, or yet further consists of at least one glycopeptide-bound oligosaccharide having a general formulae selected from Hex1HexNAc1, HexNAc2, Hex1HexNAc1Sul1, HexNAc1deHex1Sul1, HexNAc2Sul1, NeuAc1HexNAc1, Hex2HexNAc1, Hex1HexNAc1deHex1, Hex1HexNAc2, HexNAc2deHex1, Hex1HexNAc1deHex1Sul1, Hex1HexNAcSul1, Hex2HexNAc1deHex1, NeuAc1Hex1HexNAc1, Hex1HexNAc1deHex2, Hex2HexNAc2, Hex1HexNAc2deHex1, Hex2HexNAc1deHex1Sul1, Hex1HexNAc1deHex2Sul1, Hex1HexNAc3, Hex1HexNAc2deHex1Sul1, NeuAc1Hex1HexNAc1deHex1, Hex2HexNAc2Sul1, NeuAc1Hex1HexNAc2, Hex1HexNAc3Sul1, Hex2HexNAc2deHex1, Hex1HexNAc2deHex2, Hex1HexNAc3deHex1, Hex2HexNAc3, Hex2HexNAc2deHex1Sul1, Hex1HexNAc4, Hex1HexNAc3deHex1Sul1, Hex3HexNAc2deHex1, NeuAc1Hex2HexNAc2, Hex2HexNAc2deHex2, Hex2HexNAc3deHex1, Hex3HexNAc2deHex1Sul1, Hex2HexNAc2deHex2Sul1, Hex2HexNAc4, Hex2HexNAc3deHex1Sul1, Hex3HexNAc3deHex1, Hex2HexNAc4deHex1, Hex3HexNAc3deHex1Sul1, and Hex4HexNAc3deHex1Sul1. In some embodiments, the composition comprises, or alternatively consists essentially of, or yet further consists of at least one or both of NeuAc1HexNAc1 and NeuAc1Hex1HexNAc1.

In some embodiments, the composition administered to the donor mammal comprises, or alternatively consists essentially of, or yet further consists of glycopeptide-bound oligosaccharides having at least 10, at least 20, at least 30, at least 40, at least 44, or all of the general formulae provided above (e.g., the composition comprises, or alternatively consists essentially of, or yet further consists of at least 10, 20, 30, 40, 44 glycopeptides, wherein each of the glycopeptides comprises, or alternatively consists essentially of, or yet further consists of a different glycopeptide-bound oligosaccharide from the above list). In some embodiments, the composition comprises, or alternatively consists essentially of, or yet further consists of glycopeptide-bound oligosaccharides having at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or all of the general formulae provided above.

In some embodiments, the composition administered to the donor mammal comprises, or alternatively consists essentially of, or yet further consists of at least one glycopeptide-bound oligosaccharide having a general formulae selected from Hex1HexNAc1, Hex1HexNAc1deHex1, Hex1HexNAc1deHex1Sul1, Hex1HexNAc1deHex2, Hex1HexNAc1deHex2Sul1, Hex1HexNAc1Sul1, Hex1HexNAc2, Hex1HexNAc2deHex1, Hex1HexNAc2deHex1Sul1, Hex1HexNAc2deHex2, Hex1HexNAc2Sul1, Hex1HexNAc3, Hex1HexNAc3deHex1, Hex1HexNAc3deHex1Sul1, Hex1HexNAc3Sul1, Hex1HexNAc4, Hex1HexNAcSul1, Hex2HexNAc1, Hex2HexNAc1deHex1, Hex2HexNAc1deHex1Sul1, Hex2HexNAc2, Hex2HexNAc2deHex1, Hex2HexNAc2deHex1Sul1, Hex2HexNAc2deHex2, Hex2HexNAc2deHex2Sul1, Hex2HexNAc2Sul1, Hex2HexNAc3, Hex2HexNAc3deHex1, Hex2HexNAc3deHex1Sul1, Hex2HexNAc4, Hex2HexNAc4deHex1, Hex3HexNAc2deHex1, Hex3HexNAc2deHex1Sul1, Hex3HexNAc3deHex1, Hex3HexNAc3deHex1Sul1, Hex4HexNAc3deHex1Sul1, HexNAc1deHex1Sul1, HexNAc2, HexNAc2deHex1, HexNAc2Sul1, NeuAc1Hex1HexNAc1, NeuAc1Hex1HexNAc1deHex1, NeuAc1Hex1HexNAc2, NeuAc1Hex2HexNAc2, and NeuAc1HexNAc1.

In some embodiments, the composition administered to the donor mammal comprises, or alternatively consists essentially of, or yet further consists of glycopeptide-bound oligosaccharides having at least 10, at least 20, at least, 30, at least 40, at least 45, or all (e.g., at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46) of the general formulae listed above. In some embodiments, the composition comprises, or alternatively consists essentially of, or yet further consists of at least 10 of the general formulae listed above, and each formula is present in the composition between 0.1% and 10% (e.g., 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%) of all glycopeptide-bound oligosaccharides in the composition.

In some embodiments, the composition administered to the donor mammal comprises, or alternatively consists essentially of, or yet further consists of at least one glycopeptide-bound oligosaccharide having a general formulae selected from Hex1HexNAc1, HexNAc2, Hex1HexNAc1Sul, Hex1NAc2Sul1, NeuAc1HexNAc1, NeuGc1HexNAc1, Hex1HexNAc1deHex1, Hex2HexNAc1, Hex1HexNAc2, HexNAc3, Hex1HexNAc2Sul1, NeuAc1Hex1HexNAc1, NeuGc1Hex1HexNAc1, Hex2HexNAc1deHex1, Hex1NAc3Sul1, NeuAc1HexNAc2, NeuGc1HexNAc2, Hex1HexNAc2deHex1, Hex2HexNAc2, Hex1HexNAc3, Hex1HexNAc2deHex1Sul1, NeuAc1Hex1HexNAc1deHex1, Hex2HexNAcSul1, NeuGc1Hex1HexNAc1deHex1, Hex1HexNAc3Sul1, NeuAc1Hex1HexNAc2, NeuGc1Hex1HexNAc2, Hex2HexNAc2deHex1, Hex3HexNAc2, Hex1HexNAc3deHex1, Hex2HexNAc3, NeuAc1Hex1HexNAc2Sul1, NeuAc2Hex1HexNAc1, NeuGc1Hex1HexNAc2Sul1, Hex2HexNAc2deHex1Sul1, NeuAc1NeuGc1Hex1HexNAc1, NeuGc2Hex1HexNAc1, Hex1HexNAc3deHex1Sul1, NeuAc1Hex1HexNAc2deHex1, Hex2HexNAc3Sul1, NeuGc1Hex1HexNAc2deHex1, NeuAc1Hex2HexNAc2, Hex2HexNAc2deHex2, NeuGc1Hex2HexNAc2, Hex2HexNAc3deHex1, NeuAc1Hex2HexNAc2Sul1, Hex2HexNAc2deHex2Sul1, NeuGc1Hex2HexNAc2Sul1, Hex1HexNAc4deHex1, NeuAc1Hex1HexNAc3Sul1, Hex1HexNAc3deHex2Sul1, NeuAc2Hex1HexNAc2, NeuGc1Hex1HexNAc3Sul1, Hex2HexNAc3deHex1Sul1, NeuAc1Hex2HexNAc2deHex1, NeuGc2Hex1HexNAc2, NeuGc1Hex2HexNAc2deHex1, NeuGc1Hex3HexNAc2, Hex1HexNAc4deHex1Sul1, NeuAc1Hex1HexNAc3deHex1, Hex2HexNAc3deHex2 NeuGc1Hex2HexNAc3, NeuAc1Hex2HexNAc2deHex1Sul1, Hex2HexNAc3deHex2Sul1, NeuAc1Hex2HexNAc3deHex1Sul1, NeuGc1Hex2HexNAc3deHex1Sul1, Hex2HexNAc4deHex2Sul1, Hex2HexNAc5deHex2Sul1, NeuAc1Hex5HexNAc4deHex1, and NeuAc1Hex4HexNAc4deHex2Sul1.

In some embodiments, the composition administered to the donor mammal comprises, or alternatively consists essentially of, or yet further consists of glycopeptide-bound oligosaccharides having at least 10, at least 20, at least, 30, at least 40, at least 45, at least 50, at least 55, at least 60, at least 65, (e.g., at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65) or all of the general formulae listed above. In some embodiments, the composition comprises, or alternatively consists essentially of, or yet further consists of at least 10 of the general formulae listed above, and each formula is present in the composition between 0.1% and 10% (e.g., 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%) of all glycopeptide-bound oligosaccharides in the composition. In some embodiments, the composition comprises, or alternatively consists essentially of, or yet further consists of at least one glycopeptide-bound oligosaccharide having a structure selected from Galβ1-3GalNAcol, GlcNAcβ1-4Galol, GlcNAcα1-4Galol, HexNAc-GlcAol, GlcNAcβ1-6GalNAcol, Galβ1-4(6S)GlcNAcol, 6SGlcNAc-Fucol, Galβ1-4(6S)GlcNAcol, (S)Galβ1-GlcNAcol, (S)Galβ1-GlcNAcol, 6SGlcNAcβ1-6GalNAcol, 6SGlcNAcβ1-3GalNAcol, NeuAc-HexNAcol, NeuAcα2-6GalNAcol, Galβ1-4GlcNAcβ1-3Gal minus H₂O, Fucα1-2(GalNAcβ1-3)Galol, Gal-GlcNAc-Fucol, Gal-GlcNAc-Fucol, Fucα1-2Galβ1-4GlcNAcol, Fucα1-2Galβ1-3GlcNAcol, Fucα1-2Galβ1-3GalNAcol, Galβ1-4GlcNAcβ1-3Gal, GlcNAcα1-4Galβ1-4GlcNAc minus H₂O, GlcNAc-GlcNAc-Fucol, Galβ1-3(GlcNAcβ1-6)GalNAcol, GlcNAcα1-4Galβ1-3GalNAcol, GlcNAcα1-4Galβ1-4GlcNAcol, Fucα1-2Galβ1-4(6S)GlcNAcol, Galβ1-4(Fucα1-3)(6S)GlcNAcol, 6SGalβ1-3(Fucα1-4)GlcNAcol, SGalβ1-3(GlcNAcβ1-6)GalNAcol, Galβ1-3(6SGlcNAcβ1-6)GalNAcol, Galβ1-4(6S)GlcNAcβ1-6GalNAcol, Fucα1-2Galβ-4GlcNAcβ1-3Gal minus H₂O, Galβ1-3(NeuAcα2-6)GalNAcol, NeuAcα2-3Galβ1-3GalNAcol, Fucα1-2Galβ1-4(Fucα1-3)GlcNAcol, Fucα1-2Galβ-4GlcNAcβ1-3Gal, GlcNAcα1-4Galβ1-4GlcNAcβ1-3Gal minus H₂O, Fucα1-2(GalNAcα1-3)Galβ1-4GlcNAcol, Fucα1-2(GalNAcβ1-3)Galβ1-3GalNAcol, GlcNAcα1-4Gal(Fuc)GlcNAcol, GlcNAcα1-4Gal(Fuc)GlcNAcol, Fucα1-2Galβ1-3(GlcNAcβ1-6)GalNAcol, GlcNAcα1-4Galβ1-4GlcNAcβ1-3Gal, Galβ1-3(Galβ1-4GlcNAcβ1-6)GalNAcol, GlcNAcα1-4Galβ1-4GlcNAcβ1-3Galol, Galβ1-3(Galβ1-4GlcNAcβ1-6)GalNAcol, Galβ1-4GlcNAcβ1-3Galβ1-3GalNAcol, Fucα1-2Galβ1-4(6S)GlcNAcβ1-4Gal minus H₂O, Fucα1-2(S)Galβ1-4GlcNAcβ1-4Gal minus H2O, Fucα1-2Gal(Fuc)(6S)GlcNAcol, Fucα1-2Galβ1-3(6S)GlcNAcβ1-4Galol, GlcNAcα1-4Galβ1-3(GlcNAcβ1-6)GalNAcol, GlcNAcα1-3Galβ1-4GlcNAcβ1-6GalNAcol, Fucα1-2(GalNAcα1-3)Galβ1-4(6S)GlcNAcol, Fucα1-2Galβ1-4(6S)GlcNAcβ1-3GalNAcol, Fucα1-2Galβ1-3(6S-GlcNAcβ1-6)GalNAcol, Fucα1-2Galβ1-3(NeuAcα2-6)GalNAcol, Galβ1-3[Galβ1-4(6S)GlcNAcβ1-6]GalNAcol, GlcNAcα1-4Galβ1-3(NeuAcα2-6)GalNAcol, GlcNAcα1-4Galβ1-3[(6S)GlcNAcβ1-6]GalNAcol, GalNAcα1-3(Fucα1-2)Galβ1-4GlcNAcβ1-3Gal minus H₂O, GlcNAcα1-4Galβ1-3(NeuAcα2-6)GalNAcol, Fucα1-2(GalNAcβ1-3)Gal-(Fuc)GlcNAcol, Galβ1-3(Fucα1-4)GlcNAcβ1-3Galβ1-3GalNAcol, Galβ1-4(Fucα1-3)GlcNAcβ1-3Galβ1-3GalNAcol, Fucα1-2Galβ1-4GlcNAcβ1-3Galβ1-4GlcNAcol, Galβ1-3(Fucα1-2Galβ1-4GlcNAcβ1-6)GalNAcol, Fucα1-2Galβ1-3(Galβ1-4GlcNAcβ1-6)GalNAcol, Fucα1-2(GalNAcα1-3)Galβ1-4GlcNAcβ1-6GalNAcol, Fucα1-2Galβ1-3(GalNAcβ1-4GlcNAcβ1-6)GalNAcol, Galβ1-4GlcNAcβ1-3(GlcNAcβ1-6)Galβ1-3GalNAcol, GlcNAcα1-4Galβ1-3(Galβ1-4GlcNAcβ1-6)GalNAcol, GalNAcα1-3(Fucα1-2)Galβ1-4(6S)GlcNAcβ1-3Gal minus H₂O, Fucα1-2Galβ1-4(6S)GlcNAcβ1-3Galβ1-3GalNAcol, Fucα1-2Galβ1-3[Galβ1-4(6S)GlcNAcβ1-6]GalNAcol, GlcNAcα1-4Galβ1-3(GalNAcβ1-4GlcNAcβ1-6)GalNAcol, GlcNAcβ1-3[Fucα1-2Galβ1-4(6S)GlcNAcβ1-6]GalNAcol, GalNAcα1-3(Fucα1-2)Galβ1-3(6SGlcNAcβ1-6)GalNAcol, Fucα1-2Galβ1-4GlcNAcβ1-3(Galβ1-4GlcNAcβ1-6)Gal, Fucα1-2Galβ1-4GlcNAcβ1-3(Galβ1-4GlcNAcβ1-6)Gal, Galβ1-4GlcNAcβ1-3Galβ1-3(NeuAcα2-6)GalNAcol, Fucα1-2Galβ1-3(Fucα1-2Galβ1-4GlcNAcβ1-6)GalNAcol, Galβ1-3[Fucα1-2(GalNAcα1-3)Galβ1-4GlcNAcβ1-6]GalNAcol, GlcNAcα1-4Galβ1-3(Fucα1-2Galβ1-4GlcNAcβ1-6)GalNAcol, Fucα1-2Galβ1-3(GlcNAcα1-4Galβ1-4GlcNAcβ1-6)GalNAcol, Fucα1-2Galβ1-4(6S)GlcNAcβ1-3(Galβ1-4GlcNAcβ1-6)Gal minus H₂O, Fucα1-2Galβ1-3[Fucα1-2Galβ1-4(6S)GlcNAcβ1-6]GalNAcol, GlcNAcα1-4Galβ1-3(GlcNAcα1-4Galβ1-4GlcNAcβ1-6)GalNAcol, Gal-GlcNAcβ1-3(GlcNAcα1-4Galβ1-4GlcNAcβ1-6)GalNAcol, GalNAcα1-3(Fucα1-2)Galβ1-4(6S)GlcNAcβ1-3Galβ1-3GalNAcol, Fucα1-2Galβ1-4GlcNAcβ1-3Galβ1-4GlcNAcβ1-3Galβ1-3GalNAcol, GlcNAcα1-4Galβ1-3[GalNAcα1-3(Fucα1-2)Galβ1-4GlcNAcβ1-6]GalNAcol, Fucα1-2Galβ1-4(6S)GlcNAcβ1-3Galβ1-4GlcNAcβ1-3Galβ1-4GlcNAcol, and Fucα1-2Galβ1-4(6S)GlcNAcβ1-3Galβ1-4GlcNAcβ1-3(Galβ1-4GlcNAcβ1-6)Gal minus H₂O. In some embodiments, the composition comprises, or alternatively consists essentially of, or yet further consists of Galβ1-3GalNAcol (T antigen) and NeuAcα2-3Galβ1-3GalNAcol (Sialyl T antigen). In some embodiments, the composition comprises, or alternatively consists essentially of, or yet further consists of Galβ1-3(Fucα1-4)GlcNAcβ1-3Galβ1-3GalNAcol (Lewis A) and Galβ1-4(Fucα1-3)GlcNAcβ1-3Galβ1-3GalNAcol (Lewis X).

In some embodiments, the administered to the donor mammal composition comprises, or alternatively consists essentially of, or yet further consists of glycopeptide-bound oligosaccharides having at least 10, at least 20, at least 50, at least 75, or all of the structures provided above (e.g., the composition comprises, or alternatively consists essentially of, or yet further consists of at least 10, 20, 50, 75, or all of the glycopeptides, wherein each of the glycopeptides comprises, or alternatively consists essentially of, or yet further consists of a different glycopeptide-bound oligosaccharide from the above list). In some embodiments, the composition comprises, or alternatively consists essentially of, or yet further consists of glycopeptide-bound oligosaccharides having at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or all of the structures provided above.

In some embodiments, the composition comprises, or alternatively consists essentially of, or yet further consists of glycopeptide-bound oligosaccharides having at least 10, at least 20, at least 50, at least 75, or all of the structures provided above (e.g., the composition comprises, or alternatively consists essentially of, or yet further consists of at least 10, 20, 50, 75, or 99 glycopeptides, wherein each of the glycopeptides comprises, or alternatively consists essentially of, or yet further consists of a different glycopeptide-bound oligosaccharide from the above list).

In some embodiments, the composition has a salt content of less than between about 5% and 1%. In some embodiments, the composition has a salt content of less than about 2%. In some embodiments, the composition has a salt content of less than about 1%.

In some embodiments, the composition has a pH of less than 7.5 (e.g., pH 7.4, 7.3, 7.2, 7.1, 7, 6.9, 6.8, 6.7, 6.6, 6.5, 6.4, 6.3, 6.2, 6.1, or 6).

In some embodiments, the composition does not comprise more than about 5%, more than about 4%, more than about 3%, more than about 2%, more than about 1%, more than about 0.5%, or more than about 0.1% free glycans by weight. In some embodiments, the composition has a free glycan content of less than 1% by weight. In some embodiments, the composition has a free glycan content of less than 0.1% by weight. In some embodiments, the composition has a free glycan content of substantially zero. The phrase “free glycans” refers to glycans that are not attached to a protein or polypeptide.

In some embodiments, the composition comprising a mixture of glycopeptides is obtained from porcine intestinal mucins or a partially purified fraction thereof, wherein: the composition is obtained without subjecting the mucins or the partially purified fraction thereof to conditions or reagents that release oligosaccharides from glycopeptides; the composition has a total glycan content of greater than 10%; the composition comprises, or alternatively consists essentially of, or yet further consists of glycopeptide-bound oligosaccharides having each of the following general formulae: Hex1HexNAc1, HexNAc2, NeuAc1HexNAc1, NeuGc1HexNAc1, Hex1HexNAc1Fuc1, Hex1HexNAc2, Hex1HexNAc2Sul1, NeuAc1Hex1HexNAc1, NeuGc1Hex1HexNAc1, NeuAc1HexNAc2, NeuGc1HexNAc2, Hex1HexNAc2Fuc1, Hex1HexNAc2Fuc1Sul1, NeuAc1Hex1HexNAc1Fuc1, Hex1HexNAc3Sul1, Hex2HexNAc2Fuc1, Hex1HexNAc3Fuc1Sul1, Hex2HexNAc2Fuc2Sul1, and the composition does not substantially contain insoluble particles having a diameter greater than 7 μm or compounds having a molecular weight of greater than about 3 kDa.

In some embodiments, the composition comprises, or alternatively consists essentially of, or yet further consists of glycopeptide-bound oligosaccharides having each of the following general formulae: Hex2HexNAc3deHex1Sul2, Hex2HexNAc3deHex1Sul2, NeuGc1Hex2HexNAc3, NeuAc1Hex2HexNAc2deHex1Sul1, Hex2HexNAc4Sul2, Hex2HexNAc4Sul2, NeuAc1Hex2HexNAc3Sul1, Hex2HexNAc3deHex2Sul1, Hex2HexNAc3deHex2Sul1, Hex2HexNAc3deHex2Sul1, Hex2HexNAc3deHex2Sul1, Hex2HexNAc3deHex1Sul3, NeuGc1Hex2HexNAc3Sul1, NeuGc1Hex2HexNAc3Sul1, Hex2HexNAc3deHex2Sul2, Hex2HexNAc3deHex2Sul2, Hex2HexNAc3deHex2Sul2, Hex2HexNAc3deHex2Sul2, NeuGc1Hex2HexNAc3deHex1, NeuGc1Hex2HexNAc3Sul2, Hex3HexNAc3deHex1Sul2, NeuGc1Hex2HexNAc2deHex2Sul1, Hex2HexNAc4deHex1Sul2, NeuAc1Hex2HexNAc3deHex1Sul1, Hex2HexNAc3deHex3Sul1, NeuGc1Hex2HexNAc3deHex1Sul1, NeuGc1Hex2HexNAc3deHex1Sul1, Hex3HexNAc3deHex2Sul1, NeuGc1Hex2HexNAc2deHex2Sul2, NeuGc1Hex3HexNAc2deHex1Sul2, Hex2HexNAc4deHex2Sul1, Hex2HexNAc4deHex2Sul1, Hex2HexNAc4deHex2Sul1, NeuGc1Hex2HexNAc4Sul1, Hex3Hex4deHex1Sul1, Hex2HexNAc3deHex3Sul2, Hex2HexNAc3deHex3Sul2, NeuGc1Hex2HexNAc3deHex1Sul2, NeuGc1Hex2HexNAc3deHex1Sul2, NeuGc1Hex2HexNAc3deHex1Sul2, NeuGc1Hex3HexNAc2deHex2Sul1, NeuAc1Hex2HexNAc4deHex1, Hex2HexNAc4deHex2Sul2, Hex2HexNAc4deHex2Sul2, NeuGc1Hex2HexNAc4deHex1, Hex2HexNAc3deHex4Sul1, Hex3HexNAc4deHex1Sul2, NeuGc1Hex2HexNAc3deHex2Sul1, NeuGc1Hex2HexNAc3deHex2Sul1, NeuGc1Hex3HexNAc3deHex1Sul1, NeuGc1Hex2HexNAc2deHex3Sul2, Hex2HexNAc4deHex3Sul1, NeuGc1Hex2HexNAc4deHex1Sul1, NeuGc1Hex2HexNAc4deHex1Sul1, NeuGc1Hex2HexNAc4deHex1Sul1, NeuGc1Hex2HexNAc4deHex1Sul1, NeuGc1Hex2HexNAc3deHex2Sul2, Hex3HexNAc3deHex3Sul2, NeuGc1Hex3HexNAc3deHex1Sul2, Hex2HexNAc5deHex2Sul1, Hex2HexNAc4deHex3Sul2, NeuGc1Hex2HexNAc4deHex2Sul1, NeuGc1Hex2HexNAc4deHex2Sul1, NeuGc1Hex3HexNAc4deHex1Sul1, Hex2HexNAc5deHex3Sul1, NeuGc1Hex2HexNAc4deHex2Sul2, NeuGc1Hex2HexNAc4deHex2Sul2, NeuGc1Hex2HexNAc4deHex2Sul2, NeuGc1Hex3HexNAc4deHex1Sul2, Hex3HexNAc4deHex4Sul2, NeuAc1Hex5HexNAc4deHex1, NeuAc1Hex4HexNAc4deHex2Sul1, NeuGc1Hex3HexNAc4deHex4Sul1, NeuGc1Hex4HexNAc4deHex3Sul1, and NeuGc1Hex3HexNAc4deHex4Sul2. In some embodiments, the composition comprises, or alternatively consists essentially of, or yet further consists of at least one, two or all three of NeuAc1HexNAc1, NeuAc2Hex1HexNAc1, and NeuAc1Hex1HexNAc1.

In some embodiments, the sialic acid content of the composition is greater than 5%, 10%, 15%, 20%, 22.5%, 25%, 27.5%, 30%, 32.5%, 35%, or 40% (w/w). In some embodiments, the sialic acid content of the composition is greater than 25% (w/w) and less than 50% (e.g., 25, 30, 35, 40, 45, or 49%). In some embodiments, sialic acid content is determined by LC-MS/MS.

In some embodiments, the composition comprises, or alternatively consists essentially of, or yet further consists of glycopeptide-bound oligosaccharides having at least 7, 14, 21 or all 28 of the following structures: Galβ1-3GalNAc, GlcNAcβ1-6GalNAc, NeuAcα2-6GalNAc, NeuGcα2-6GalNAc, Fucα1-2Galβ1-3GalNAc, Gal+GlcNAcβ1-6GalNAc, Galβ1-3(GlcNAcβ1-6)GalNAc, Galβ1-3GlcNAcβ1-6GalNAc, Galβ1-3(GlcNAcβ1-6)GalNAc, Galβ1-3(6SGlcNAcβ1-6)GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, GlcNAc-(NeuAcα2-6)GalNAc, GalNAc-(NeuAcα2-6)GalNAc, HexNAc-(NeuGcα2-6)GalNAc, Fucα1-2(GalNAcβ1-3)Galβ1-3GalNAc, Fucα1-2Galβ1-4GlcNAcβ1-6GalNAc, Fucα1-2Galβ1-3(GlcNAcβ1-6)GalNAc, Fucα1-2Galβ1-3(6S-GlcNAcβ1-6)GalNAc, Fucα1-2Gal P1-3(NeuAcβ2-6)GalNAc, GlcNAcβ1-3[Galβ1-4(6S)GlcNAcβ1-6]GalNAc, Galβ1-4GlcNAcβ1-3[(6S)GlcNAcβ1-6]GalNAc, Galβ1-3(Fucα1-2Galβ1-4GlcNAcβ1-6)GalNAc, Fucα1-2Galβ1-4(6S)GlcNAcβ1-6[GlcNAcβ1-3]GalNAc, GlcNAcβ1-3[Fucα1-2Galβ1-3(6S-)GlcNAcβ1-6]GalNAc, and Fucα1-2Galβ1-3[Fucα1-2Galβ1-4(6S)GlcNAcβ1-6]GalNAc.

In some embodiments, the composition comprises, or alternatively consists essentially of, or yet further consists of at least 1, 2, 3, 4, 5, 6, 7, 8, 9, or all 10 sialylated glycopeptide-bound oligosaccharide selected from the following: NeuAcα2-6GalNAc, NeuGcα2-6GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, GlcNAc-(NeuAcα2-6)GalNAc, GalNAc-(NeuAcα2-6)GalNAc, HexNAc-(NeuGcα2-6)GalNAc, and Fucα1-2Galβ1-3(NeuAcβ2-6)GalNAc. In some embodiments, the composition comprises, or alternatively consists essentially of, or yet further consists of NeuAcα2-3Galβ1-3GalNAc (Sialyl-T antigen), NeuAcα2-6GalNAc (Sialyl Tn antigen), and NeuAcα2-3Galβ1-3(NeuAcα2-6)GalNAc (Disialyl T antigen).

### Methods for Preparing Reprogrammed T Cells

Another aspect of the disclosure is directed to a method of preparing reprogrammed T cells comprising, or alternatively consisting essentially of, or yet further consisting of administering to a donor mammal an effective amount of a composition comprising, or alternatively consisting essentially of, or yet further consisting of a plurality of glycopeptides, wherein at least 90%, at least 91%, at least 92%, at least 93%, at least 94%, at least 95%, at least 96%, at least 97%, at least 98%, at least 99%, or all of the plurality of glycopeptides in the composition are less than or equal to 2.2 KDa but greater than or equal to 0.5 KDa in size (e.g., 2.2, 2.1, 2, 1.9, 1.8, 1.7, 1.6, 1.5, 1.4, 1.3, 1.2, 1.1, 1, 0.9, 0.8, 0.7, 0.6, or 0.5 KDa, or any value therebetween) and wherein the plurality of glycopeptides comprises, or alternatively consists essentially of, or yet further consists of the following oligosaccharide structures: Fucα1-2Galβ1-3GalNAc, Galβ1-3(GlcNAcβ1-6)GalNAc, Galβ1-3(6SGlcNAcβ1-6)GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, Galβ1-3(Galβ1-4GlcNAcβ1-6)GalNAc, Fucα1-2Galβ1-3(6S-GlcNAcβ1-6)GalNAc, Galβ1-3(Galα1-3Galβ1-4GlcNAcβ1-6)GalNAc, and NeuAcα2-3Galβ1-3[(6S)GlcNAcβ1-6]GalNAc; thereby treating the cancer in the recipient mammal.

Another aspect of the disclosure is directed to a method of preparing reprogrammed T cells comprising, or alternatively consisting essentially of, or yet further consisting of administering to a donor mammal an effective amount of a composition comprising, or alternatively consisting essentially of, or yet further consisting of a plurality of glycopeptides, wherein at least 90%, at least 91%, at least 92%, at least 93%, at least 94%, at least 95%, at least 96%, at least 97%, at least 98%, at least 99%, or all of the plurality of glycopeptides in the composition comprise less than 15 amino acids greater than or equal to 8 amino acids (e.g., 14, 13, 12, 11, 10, 9 or 8 amino acids), and wherein the plurality of glycopeptides comprises, or alternatively consists essentially of, or yet further consists of the following oligosaccharide structures: Fucα1-2Galβ1-3GalNAc, Galβ1-3(GlcNAcβ1-6)GalNAc, Galβ1-3(6SGlcNAcβ1-6)GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, Galβ1-3(Galβ1-4GlcNAcβ1-6)GalNAc, Fucα1-2Galβ1-3(6S-GlcNAcβ1-6)GalNAc, Galβ1-3(Galα1-3Galβ1-4GlcNAcβ1-6)GalNAc, and NeuAcα2-3Galβ1-3[(6S)GlcNAcβ1-6]GalNAc; thereby treating the cancer in the recipient mammal.

In some embodiments, the method further comprises cryopreserving the isolated T cells.

In some embodiments, the T cells are HLA1-deficient T cells. In some embodiments, the T cells are HLA1-deficient T cells TCR-deficient T cells. In some embodiments, the T cells are genetically engineered to lack HLA1 (Human Leukocyte Antigen 1) and/or TCR (T cell receptor). In some embodiments, the T cells are universal T cells as described in Zhao, Juanjuan, et al. (Journal of hematology & oncology 11 (2018): 1-9), which is incorporated herein in its entirety.

In some embodiments, the composition is administered to the donor mammal for 28 days or more (e.g., between 28 and 40 days) before isolating the T cells.

In some embodiments, the T cells isolated from the donor animal are cultured and expanded in vitro before administering the T cells to the recipient mammal.

In some embodiments, the plurality of glycopeptides is derived from porcine gastrointestinal mucins, and wherein the composition is obtained without subjecting the porcine gastrointestinal mucins to conditions or reagents that cause release of oligosaccharides from glycopeptides.

In some embodiments, the composition administered to the donor mammal comprises, or alternatively consists essentially of, or yet further consists of between about 0.5-1% (e.g., 0.5, 0.6, 0.7, 0.8, 0.9, 1% or any value therebetween) Fucα1-2Galβ1-3GalNAc containing glycopeptides by weight of the total composition. In some embodiments, the composition administered to the donor mammal comprises, or alternatively consists essentially of, or yet further consists of between about 0.5-1% (e.g., 0.5, 0.6, 0.7, 0.8, 0.9, 1% or any value therebetween) Galβ1-3(GlcNAcβ1-6)GalNAc containing glycopeptides by weight of the total composition. In some embodiments, the composition administered to the donor mammal comprises, or alternatively consists essentially of, or yet further consists of between about 3-5% (e.g., 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5 or any value therebetween) Galβ1-3(6SGlcNAcβ1-6)GalNAc containing glycopeptides by weight of the total composition. In some embodiments, the composition administered to the donor mammal comprises, or alternatively consists essentially of, or yet further consists of between about 1-3% (e.g., 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3 or any value therebetween) Galβ1-3(NeuAcα2-6)GalNAc by weight of the total composition. In some embodiments, the composition administered to the donor mammal comprises, or alternatively consists essentially of, or yet further consists of between about 1-3% % (e.g., 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3 or any value therebetween) NeuAcα2-3Galβ1-3GalNAc containing glycopeptides by weight of the total composition. In some embodiments, the composition administered to the donor mammal comprises, or alternatively consists essentially of, or yet further consists of between about 1-3% % (e.g., 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3 or any value therebetween) Galβ1-3(NeuGcα2-6)GalNAc containing glycopeptides by weight of the total composition. In some embodiments, the composition administered to the donor mammal comprises, or alternatively consists essentially of, or yet further consists of between about 1-3% (e.g., 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3 or any value therebetween) NeuGcα2-3Galβ1-3GalNAc containing glycopeptides by weight of the total composition. In some embodiments, the composition administered to the donor mammal comprises, or alternatively consists essentially of, or yet further consists of between about 0.5-1% (e.g., 0.5, 0.6, 0.7, 0.8, 0.9, 1% or any value therebetween) Galβ1-3(Galβ1-4GlcNAcβ1-6)GalNAc containing glycopeptides by weight of the total composition. In some embodiments, the composition administered to the donor mammal comprises, or alternatively consists essentially of, or yet further consists of between about 3-5% (e.g., 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5 or any value therebetween) Fucα1-2Galβ1-3(6S-GlcNAcβ1-6)GalNAc containing glycopeptides by weight of the total composition. In some embodiments, the composition administered to the donor mammal comprises, or alternatively consists essentially of, or yet further consists of between about 0.5-1% (e.g., 0.5, 0.6, 0.7, 0.8, 0.9, 1% or any value therebetween) Galβ1-3(Galα1-3Galβ1-4GlcNAcβ1-6)GalNAc containing glycopeptides by weight of the total composition. In some embodiments, the composition administered to the donor mammal comprises, or alternatively consists essentially of, or yet further consists of between about 0.5-1% (e.g., 0.5, 0.6, 0.7, 0.8, 0.9, 1% or any value therebetween) NeuAcα2-3Galβ1-3[(6S)GlcNAcβ1-6]GalNAc containing glycopeptides by weight of the total composition.

In some embodiments, the composition administered to the donor mammal comprises, or alternatively consists essentially of, or yet further consists of between about 0.5-1% Fucα1-2Galβ1-3GalNAc containing glycopeptides, between about 0.5-1% Galβ1-3(GlcNAcβ1-6)GalNAc containing glycopeptides, between about 3-5% Galβ1-3(6SGlcNAcβ1-6)GalNAc containing glycopeptides, between about 1-3% Galβ1-3(NeuAcα2-6)GalNAc, between about 1-3% NeuAcα2-3Galβ1-3GalNAc containing glycopeptides, between about 1-3% Galβ1-3(NeuGcα2-6)GalNAc containing glycopeptides, between about 1-3% NeuGcα2-3Galol-3GalNAc containing glycopeptides, between about 0.5-1% Galβ1-3(Galβ1-4GlcNAcβ1-6)GalNAc containing glycopeptides, between about 3-5% Fucα1-2Galβ1-3(6S-GlcNAcβ1-6)GalNAc containing glycopeptides, between about 0.5-1% Galβ1-3(Galα1-3Galβ1-4GlcNAcβ1-6)GalNAc containing glycopeptides, and between about 0.5-1% NeuAcα2-3Galβ1-3[(6S)GlcNAcβ1-6]GalNAc containing glycopeptides by weight of the total composition.

In some embodiments, the composition administered to the donor mammal comprises, or alternatively consists essentially of, or yet further consists of less than about 0.1%, 0.5%, 1%, 5%, 10%, 12%, 14%, 15%, 16%, 17%, 18%, 19%, 20%, 21%, 22%, 23%, or 24% free glycans (e.g., glycans not bond to peptides or proteins). In some embodiments, the composition has a free glycan content of less than 0.1% by weight. In some embodiments, the composition comprises substantially no free glycans.

In some embodiments, the composition administered to the donor mammal contains less than 5%, 4%, 3%, 2% o 1% of insoluble particles having a diameter greater than 7 μm. In some embodiments, the composition contains substantially no insoluble particles having a diameter greater than 7 μm. In some embodiments, the diameter of at least 95%, 96%, 97%, 98%, 99% or more of insoluble particles in the composition is less than or equal to 7 μm (e.g., 7, 6, 5, 4, 3, 2, 1 μm or smaller).

In some embodiments, the composition administered to the donor mammal comprises, or alternatively consists essentially of, or yet further consists of at least 1, 2, 3, 4, 5, 6, 7, 8, 9, 10, 11, 12, 13, 14, 15, 16, 20, 25, 30, or more sialylated glycopeptide-bound oligosaccharides (i.e., 1, 2, 3, etc. different sialylated glycopeptide-bound oligosaccharide structures). In some embodiments, the sialic acid content of the composition is greater than 5%, 10%, 15%, 20%, 22.5%, 25%, 27.5%, 30%, 32.5%, 35%, or 40% (w/w).

In some embodiments, the total glycoprotein content of the composition administered to the donor mammal is 12% or less (w/w). In some embodiments, the total glycoprotein content of the composition (w/w) is less than about 11%, 10%, 9%, 8%, 7%, 6%, 5%, 4%, 3%, 2%, 1%, 0.5%, or 0.1%. In some embodiments, the composition administered to the donor mammal comprises substantially no glycoproteins.

In some embodiments, the composition is administered to the donor mammal orally or rectally.

The composition administered to the donor mammal may be obtained from mucins from any suitable source. In some embodiments, the mucins are obtained from porcine gastrointestinal tract. In some embodiments, the mucins are obtained from porcine stomach. In some embodiments, the compositions are obtained by the methods provided in Examples 1-3 herein.

In some embodiments, the composition administered to the donor mammal is obtained from porcine gastric mucins, wherein: the composition is obtained without subjecting the mucins or a partially purified fraction thereof to conditions or reagents that cause substantial release of oligosaccharides from glycopeptides; the total oligosaccharide content of the composition is >10% (w/w); the ratio of glycopeptides:free glycans is >4:1 (w/w); and the total glycoprotein content of the composition is 12% or less (w/w).

In some embodiments, the total oligosaccharide content of the composition administered to the donor mammal is greater than about 10%, 12%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 50%, or 55% (w/w). As used herein, the total oligosaccharide content is the total weight of oligosaccharide in the composition. Such weight does not include the weight of proteins or peptides attached to the oligosaccharides. In some embodiments, the oligosaccharide content of the composition is greater than about 10% (w/w). In some embodiments, the oligosaccharide content of the composition is greater than or equal to about 15% (w/w). In some embodiments, the oligosaccharide content of the composition is greater than or equal to about 20% (w/w). In some embodiments, the oligosaccharide content comprises, or alternatively consists essentially of, or yet further consists of substantially all oligosaccharides bound to glycoprotein or glycopeptide without substantially any unbound oligosaccharides. Methods of determining oligosaccharide content are known in the art and are not limited. In some embodiments, oligosaccharide content is determined by HPAEC-PAD with an acid pre-treatment to hydrolyze the glycans into monosaccharides.

In some embodiments, the ratio of glycopeptides:free glycans in the composition administered to the donor mammal is >4:1 (w/w). In some embodiments, the ratio of glycopeptides:free glycans (w/w) is about 4:1, about 5:1, about 6:1, about 7:1, about 8:1, about 9:1, about 10:1, about 11:1, about 12:1, about 13:1, about 14:1, about 15:1, about 20:1, or more.

In some embodiments, the composition administered to the donor mammal comprises, or alternatively consists essentially of, or yet further consists of at least one glycopeptide-bound oligosaccharide having a general formulae selected from Hex1HexNAc1, HexNAc2, Hex1HexNAc1Sul1, HexNAc1deHex1Sul1, HexNAc2Sul1, NeuAc1HexNAc1, Hex2HexNAc1, Hex1HexNAc1deHex1, Hex1HexNAc2, HexNAc2deHex1, Hex1HexNAc1deHex1Sul1, Hex1HexNAcSul1, Hex2HexNAc1deHex1, NeuAc1Hex1HexNAc1, Hex1HexNAc1deHex2, Hex2HexNAc2, Hex1HexNAc2deHex1, Hex2HexNAc1deHex1Sul1, Hex1HexNAc1deHex2Sul1, Hex1HexNAc3, Hex1HexNAc2deHex1Sul1, NeuAc1Hex1HexNAc1deHex1, Hex2HexNAc2Sul1, NeuAc1Hex1HexNAc2, Hex1HexNAc3Sul1, Hex2HexNAc2deHex1, Hex1HexNAc2deHex2, Hex1HexNAc3deHex1, Hex2HexNAc3, Hex2HexNAc2deHex1Sul1, Hex1HexNAc4, Hex1HexNAc3deHex1Sul1, Hex3HexNAc2deHex1, NeuAc1Hex2HexNAc2, Hex2HexNAc2deHex2, Hex2HexNAc3deHex1, Hex3HexNAc2deHex1Sul1, Hex2HexNAc2deHex2Sul1, Hex2HexNAc4, Hex2HexNAc3deHex1Sul1, Hex3HexNAc3deHex1, Hex2HexNAc4deHex1, Hex3HexNAc3deHex1Sul1, and Hex4HexNAc3deHex1Sul1. In some embodiments, the composition comprises, or alternatively consists essentially of, or yet further consists of at least one or both of NeuAc1HexNAc1 and NeuAc1Hex1HexNAc1.

In some embodiments, the composition administered to the donor mammal comprises, or alternatively consists essentially of, or yet further consists of glycopeptide-bound oligosaccharides having at least 10, at least 20, at least 30, at least 40, at least 44, or all of the general formulae provided above (e.g., the composition comprises, or alternatively consists essentially of, or yet further consists of at least 10, 20, 30, 40, 44 glycopeptides, wherein each of the glycopeptides comprises, or alternatively consists essentially of, or yet further consists of a different glycopeptide-bound oligosaccharide from the above list). In some embodiments, the composition comprises, or alternatively consists essentially of, or yet further consists of glycopeptide-bound oligosaccharides having at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or all of the general formulae provided above.

In some embodiments, the composition administered to the donor mammal comprises, or alternatively consists essentially of, or yet further consists of at least one glycopeptide-bound oligosaccharide having a general formulae selected from Hex1HexNAc1, Hex1HexNAc1deHex1, Hex1HexNAc1deHex1Sul1, Hex1HexNAc1deHex2, Hex1HexNAc1deHex2Sul1, Hex1HexNAc1Sul1, Hex1HexNAc2, Hex1HexNAc2deHex1, Hex1HexNAc2deHex1Sul1, Hex1HexNAc2deHex2, Hex1HexNAc2Sul1, Hex1HexNAc3, Hex1HexNAc3deHex1, Hex1HexNAc3deHex1Sul1, Hex1HexNAc3Sul1, Hex1HexNAc4, Hex1HexNAcSul1, Hex2HexNAc1, Hex2HexNAc1deHex1, Hex2HexNAc1deHex1Sul1, Hex2HexNAc2, Hex2HexNAc2deHex1, Hex2HexNAc2deHex1Sul1, Hex2HexNAc2deHex2, Hex2HexNAc2deHex2Sul1, Hex2HexNAc2Sul1, Hex2HexNAc3, Hex2HexNAc3deHex1, Hex2HexNAc3deHex1Sul1, Hex2HexNAc4, Hex2HexNAc4deHex1, Hex3HexNAc2deHex1, Hex3HexNAc2deHex1Sul1, Hex3HexNAc3deHex1, Hex3HexNAc3deHex1Sul1, Hex4HexNAc3deHex1Sul1, HexNAc1deHex1Sul1, HexNAc2, HexNAc2deHex1, HexNAc2Sul1, NeuAc1Hex1HexNAc1, NeuAc1Hex1HexNAc1deHex1, NeuAc1Hex1HexNAc2, NeuAc1Hex2HexNAc2, and NeuAc1HexNAc1.

In some embodiments, the composition administered to the donor mammal comprises, or alternatively consists essentially of, or yet further consists of glycopeptide-bound oligosaccharides having at least 10, at least 20, at least, 30, at least 40, at least 45, or all (e.g., at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46) of the general formulae listed above. In some embodiments, the composition comprises, or alternatively consists essentially of, or yet further consists of at least 10 of the general formulae listed above, and each formula is present in the composition between 0.1% and 10% (e.g., 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%) of all glycopeptide-bound oligosaccharides in the composition.

In some embodiments, the composition administered to the donor mammal comprises, or alternatively consists essentially of, or yet further consists of at least one glycopeptide-bound oligosaccharide having a general formulae selected from Hex1HexNAc1, HexNAc2, Hex1HexNAc1Sul, Hex1NAc2Sul1, NeuAc1HexNAc1, NeuGc1HexNAc1, Hex1HexNAc1deHex1, Hex2HexNAc1, Hex1HexNAc2, HexNAc3, Hex1HexNAc2Sul1, NeuAc1Hex1HexNAc1, NeuGc1Hex1HexNAc1, Hex2HexNAc1deHex1, Hex1NAc3Sul1, NeuAc1HexNAc2, NeuGc1HexNAc2, Hex1HexNAc2deHex1, Hex2HexNAc2, Hex1HexNAc3, Hex1HexNAc2deHex1Sul1, NeuAc1Hex1HexNAc1deHex1, Hex2HexNAcSul1, NeuGc1Hex1HexNAc1deHex1, Hex1HexNAc3Sul1, NeuAc1Hex1HexNAc2, NeuGc1Hex1HexNAc2, Hex2HexNAc2deHex1, Hex3HexNAc2, Hex1HexNAc3deHex1, Hex2HexNAc3, NeuAc1Hex1HexNAc2Sul1, NeuAc2Hex1HexNAc1, NeuGc1Hex1HexNAc2Sul1, Hex2HexNAc2deHex1Sul1, NeuAc1NeuGc1Hex1HexNAc1, NeuGc2Hex1HexNAc1, Hex1HexNAc3deHex1Sul1, NeuAc1Hex1HexNAc2deHex1, Hex2HexNAc3Sul1, NeuGc1Hex1HexNAc2deHex1, NeuAc1Hex2HexNAc2, Hex2HexNAc2deHex2, NeuGc1Hex2HexNAc2, Hex2HexNAc3deHex1, NeuAc1Hex2HexNAc2Sul1, Hex2HexNAc2deHex2Sul1, NeuGc1Hex2HexNAc2Sul1, Hex1HexNAc4deHex1, NeuAc1Hex1HexNAc3Sul1, Hex1HexNAc3deHex2Sul1, NeuAc2Hex1HexNAc2, NeuGc1Hex1HexNAc3Sul1, Hex2HexNAc3deHex1Sul1, NeuAc1Hex2HexNAc2deHex1, NeuGc2Hex1HexNAc2, NeuGc1Hex2HexNAc2deHex1, NeuGc1Hex3HexNAc2, Hex1HexNAc4deHex1Sul1, NeuAc1Hex1HexNAc3deHex1, Hex2HexNAc3deHex2 NeuGc1Hex2HexNAc3, NeuAc1Hex2HexNAc2deHex1Sul1, Hex2HexNAc3deHex2Sul1, NeuAc1Hex2HexNAc3deHex1Sul1, NeuGc1Hex2HexNAc3deHex1Sul1, Hex2HexNAc4deHex2Sul1, Hex2HexNAc5deHex2Sul1, NeuAc1Hex5HexNAc4deHex1, and NeuAc1Hex4HexNAc4deHex2Sul1.

In some embodiments, the composition administered to the donor mammal comprises, or alternatively consists essentially of, or yet further consists of glycopeptide-bound oligosaccharides having at least 10, at least 20, at least, 30, at least 40, at least 45, at least 50, at least 55, at least 60, at least 65, (e.g., at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65) or all of the general formulae listed above. In some embodiments, the composition comprises, or alternatively consists essentially of, or yet further consists of at least 10 of the general formulae listed above, and each formula is present in the composition between 0.1% and 10% (e.g., 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%) of all glycopeptide-bound oligosaccharides in the composition. In some embodiments, the composition comprises, or alternatively consists essentially of, or yet further consists of at least one glycopeptide-bound oligosaccharide having a structure selected from Galβ1-3GalNAcol, GlcNAcβ1-4Galol, GlcNAcα1-4Galol, HexNAc-GlcAol, GlcNAcβ1-6GalNAcol, Galβ1-4(6S)GlcNAcol, 6SGlcNAc-Fucol, Galβ1-4(6S)GlcNAcol, (S)Galβ1-GlcNAcol, (S)Galβ1-GlcNAcol, 6SGlcNAcβ1-6GalNAcol, 6SGlcNAcβ1-3GalNAcol, NeuAc-HexNAcol, NeuAcα2-6GalNAcol, Galβ1-4GlcNAcβ1-3Gal minus H₂O, Fucα1-2(GalNAcβ1-3)Galol, Gal-GlcNAc-Fucol, Gal-GlcNAc-Fucol, Fucα1-2Galβ1-4GlcNAcol, Fucα1-2Galβ1-3GlcNAcol, Fucα1-2Galβ1-3GalNAcol, Galβ1-4GlcNAcβ1-3Gal, GlcNAcα1-4Galβ1-4GlcNAc minus H₂O, GlcNAc-GlcNAc-Fucol, Galβ1-3(GlcNAcβ1-6)GalNAcol, GlcNAcα1-4Galβ1-3GalNAcol, GlcNAcα1-4Galβ1-4GlcNAcol, Fucα1-2Galβ1-4(6S)GlcNAcol, Galβ1-4(Fucα1-3)(6S)GlcNAcol, 6SGalβ1-3(Fucα1-4)GlcNAcol, SGalβ1-3(GlcNAcβ1-6)GalNAcol, Galβ1-3(6SGlcNAcβ1-6)GalNAcol, Galβ1-4(6S)GlcNAcβ1-6GalNAcol, Fucα1-2Galβ-4GlcNAcβ1-3Gal minus H₂O, Galβ1-3(NeuAcα2-6)GalNAcol, NeuAcα2-3Galβ1-3GalNAcol, Fucα1-2Galβ1-4(Fucα1-3)GlcNAcol, Fucα1-2Galβ-4GlcNAcβ1-3Gal, GlcNAcα1-4Galβ1-4GlcNAcβ1-3Gal minus H₂O, Fucα1-2(GalNAcα1-3)Galβ1-4GlcNAcol, Fucα1-2(GalNAcβ1-3)Galβ1-3GalNAcol, GlcNAcα1-4Gal(Fuc)GlcNAcol, GlcNAcα1-4Gal(Fuc)GlcNAcol, Fucα1-2Galβ1-3(GlcNAcβ1-6)GalNAcol, GlcNAcα1-4Galβ1-4GlcNAcβ1-3Gal, Galβ1-3(Galβ1-4GlcNAcβ1-6)GalNAcol, GlcNAcα1-4Galβ1-4GlcNAcβ1-3Galol, Galβ1-3(Galβ1-4GlcNAcβ1-6)GalNAcol, Galβ1-4GlcNAcβ1-3Galβ1-3GalNAcol, Fucα1-2Galβ1-4(6S)GlcNAcβ1-4Gal minus H₂O, Fucα1-2(S)Galβ1-4GlcNAcβ1-4Gal minus H2O, Fucα1-2Gal(Fuc)(6S)GlcNAcol, Fucα1-2Galβ1-3(6S)GlcNAcβ1-4Galol, GlcNAcα1-4Galβ1-3(GlcNAcβ1-6)GalNAcol, GlcNAcα1-3Galβ1-4GlcNAcβ1-6GalNAcol, Fucα1-2(GalNAcα1-3)Galβ1-4(6S)GlcNAcol, Fucα1-2Galβ1-4(6S)GlcNAcβ1-3GalNAcol, Fucα1-2Galβ1-3(6S-GlcNAcβ1-6)GalNAcol, Fucα1-2Galβ1-3(NeuAcα2-6)GalNAcol, Galβ1-3[Galβ1-4(6S)GlcNAcβ1-6]GalNAcol, GlcNAcα1-4Galβ1-3(NeuAcα2-6)GalNAcol, GlcNAcα1-4Galβ1-3[(6S)GlcNAcβ1-6]GalNAcol, GalNAcα1-3(Fucα1-2)Galβ1-4GlcNAcβ1-3Gal minus H₂O, GlcNAcα1-4Galβ1-3(NeuAcα2-6)GalNAcol, Fucα1-2(GalNAcβ1-3)Gal-(Fuc)GlcNAcol, Galβ1-3(Fucα1-4)GlcNAcβ1-3Galβ1-3GalNAcol, Galβ1-4(Fucα1-3)GlcNAcβ1-3Galβ1-3GalNAcol, Fucα1-2Galβ1-4GlcNAcβ1-3Galβ1-4GlcNAcol, Galβ1-3(Fucα1-2Galβ1-4GlcNAcβ1-6)GalNAcol, Fucα1-2Galβ1-3(Galβ1-4GlcNAcβ1-6)GalNAcol, Fucα1-2(GalNAcα1-3)Galβ1-4GlcNAcβ1-6GalNAcol, Fucα1-2Galβ1-3(GalNAcβ1-4GlcNAcβ1-6)GalNAcol, Galβ1-4GlcNAcβ1-3(GlcNAcβ1-6)Galβ1-3GalNAcol, GlcNAcα1-4Galβ1-3(Galβ1-4GlcNAcβ1-6)GalNAcol, GalNAcα1-3(Fucα1-2)Galβ1-4(6S)GlcNAcβ1-3Gal minus H₂O, Fucα1-2Galβ1-4(6S)GlcNAcβ1-3Galβ1-3GalNAcol, Fucα1-2Galβ1-3[Galβ1-4(6S)GlcNAcβ1-6]GalNAcol, GlcNAcα1-4Galβ1-3(GalNAcβ1-4GlcNAcβ1-6)GalNAcol, GlcNAcβ1-3[Fucα1-2Galβ1-4(6S)GlcNAcβ1-6]GalNAcol, GalNAcα1-3(Fucα1-2)Galβ1-3(6SGlcNAcβ1-6)GalNAcol, Fucα1-2Galβ1-4GlcNAcβ1-3(Galβ1-4GlcNAcβ1-6)Gal, Fucα1-2Galβ1-4GlcNAcβ1-3(Galβ1-4GlcNAcβ1-6)Gal, Galβ1-4GlcNAcβ1-3Galβ1-3(NeuAcα2-6)GalNAcol, Fucα1-2Galβ1-3(Fucα1-2Galβ1-4GlcNAcβ1-6)GalNAcol, Galβ1-3[Fucα1-2(GalNAcα1-3)Galβ1-4GlcNAcβ1-6]GalNAcol, GlcNAcα1-4Galβ1-3(Fucα1-2Galβ1-4GlcNAcβ1-6)GalNAcol, Fucα1-2Galβ1-3(GlcNAcα1-4Galβ1-4GlcNAcβ1-6)GalNAcol, Fucα1-2Galβ1-4(6S)GlcNAcβ1-3(Galβ1-4GlcNAcβ1-6)Gal minus H₂O, Fucα1-2Galβ1-3[Fucα1-2Galβ1-4(6S)GlcNAcβ1-6]GalNAcol, GlcNAcα1-4Galβ1-3(GlcNAcα1-4Galβ1-4GlcNAcβ1-6)GalNAcol, Gal-GlcNAcβ1-3(GlcNAcα1-4Galβ1-4GlcNAcβ1-6)GalNAcol, GalNAcα1-3(Fucα1-2)Galβ1-4(6S)GlcNAcβ1-3Galβ1-3GalNAcol, Fucα1-2Galβ1-4GlcNAcβ1-3Galβ1-4GlcNAcβ1-3Galβ1-3GalNAcol, GlcNAcα1-4Galβ1-3[GalNAcα1-3(Fucα1-2)Galβ1-4GlcNAcβ1-6]GalNAcol, Fucα1-2Galβ1-4(6S)GlcNAcβ1-3Galβ1-4GlcNAcβ1-3Galβ1-4GlcNAcol, and Fucα1-2Galβ1-4(6S)GlcNAcβ1-3Galβ1-4GlcNAcβ1-3(Galβ1-4GlcNAcβ1-6)Gal minus H₂O. In some embodiments, the composition comprises, or alternatively consists essentially of, or yet further consists of Galβ1-3GalNAcol (T antigen) and NeuAcα2-3Galβ1-3GalNAcol (Sialyl T antigen). In some embodiments, the composition comprises, or alternatively consists essentially of, or yet further consists of Galβ1-3(Fucα1-4)GlcNAcβ1-3Galβ1-3GalNAcol (Lewis A) and Galβ1-4(Fucα1-3)GlcNAcβ1-3Galβ1-3GalNAcol (Lewis X).

In some embodiments, the administered to the donor mammal composition comprises, or alternatively consists essentially of, or yet further consists of glycopeptide-bound oligosaccharides having at least 10, at least 20, at least 50, at least 75, or all of the structures provided above (e.g., the composition comprises, or alternatively consists essentially of, or yet further consists of at least 10, 20, 50, 75, or all of the glycopeptides, wherein each of the glycopeptides comprises, or alternatively consists essentially of, or yet further consists of a different glycopeptide-bound oligosaccharide from the above list). In some embodiments, the composition comprises, or alternatively consists essentially of, or yet further consists of glycopeptide-bound oligosaccharides having at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or all of the structures provided above.

In some embodiments, the composition comprises, or alternatively consists essentially of, or yet further consists of glycopeptide-bound oligosaccharides having at least 10, at least 20, at least 50, at least 75, or all of the structures provided above (e.g., the composition comprises, or alternatively consists essentially of, or yet further consists of at least 10, 20, 50, 75, or 99 glycopeptides, wherein each of the glycopeptides comprises, or alternatively consists essentially of, or yet further consists of a different glycopeptide-bound oligosaccharide from the above list).

In some embodiments, the composition has a salt content of less than between about 5% and 1%. In some embodiments, the composition has a salt content of less than about 2%. In some embodiments, the composition has a salt content of less than about 1%.

In some embodiments, the composition has a pH of less than 7.5 (e.g., pH 7.4, 7.3, 7.2, 7.1, 7, 6.9, 6.8, 6.7, 6.6, 6.5, 6.4, 6.3, 6.2, 6.1, or 6).

In some embodiments, the composition does not comprise more than about 5%, more than about 4%, more than about 3%, more than about 2%, more than about 1%, more than about 0.5%, or more than about 0.1% free glycans by weight. In some embodiments, the composition has a free glycan content of less than 1% by weight. In some embodiments, the composition has a free glycan content of less than 0.1% by weight. In some embodiments, the composition has a free glycan content of substantially zero. The phrase “free glycans” refers to glycans that are not attached to a protein or polypeptide.

In some embodiments, the composition comprising a mixture of glycopeptides is obtained from porcine intestinal mucins or a partially purified fraction thereof, wherein: the composition is obtained without subjecting the mucins or the partially purified fraction thereof to conditions or reagents that release oligosaccharides from glycopeptides; the composition has a total glycan content of greater than 10%; the composition comprises, or alternatively consists essentially of, or yet further consists of glycopeptide-bound oligosaccharides having each of the following general formulae: Hex1HexNAc1, HexNAc2, NeuAc1HexNAc1, NeuGc1HexNAc1, Hex1HexNAc1Fuc1, Hex1HexNAc2, Hex1HexNAc2Sul1, NeuAc1Hex1HexNAc1, NeuGc1Hex1HexNAc1, NeuAc1HexNAc2, NeuGc1HexNAc2, Hex1HexNAc2Fuc1, Hex1HexNAc2Fuc1Sul1, NeuAc1Hex1HexNAc1Fuc1, Hex1HexNAc3Sul1, Hex2HexNAc2Fuc1, Hex1HexNAc3Fuc1Sul1, Hex2HexNAc2Fuc2Sul1, and the composition does not substantially contain insoluble particles having a diameter greater than 7 μm or compounds having a molecular weight of greater than about 3 kDa.

In some embodiments, the composition comprises, or alternatively consists essentially of, or yet further consists of glycopeptide-bound oligosaccharides having each of the following general formulae: Hex2HexNAc3deHex1Sul2, Hex2HexNAc3deHex1Sul2, NeuGc1Hex2HexNAc3, NeuAc1Hex2HexNAc2deHex1Sul1, Hex2HexNAc4Sul2, Hex2HexNAc4Sul2, NeuAc1Hex2HexNAc3Sul1, Hex2HexNAc3deHex2Sul1, Hex2HexNAc3deHex2Sul1, Hex2HexNAc3deHex2Sul1, Hex2HexNAc3deHex2Sul1, Hex2HexNAc3deHex1Sul3, NeuGc1Hex2HexNAc3Sul1, NeuGc1Hex2HexNAc3Sul1, Hex2HexNAc3deHex2Sul2, Hex2HexNAc3deHex2Sul2, Hex2HexNAc3deHex2Sul2, Hex2HexNAc3deHex2Sul2, NeuGc1Hex2HexNAc3deHex1, NeuGc1Hex2HexNAc3Sul2, Hex3HexNAc3deHex1Sul2, NeuGc1Hex2HexNAc2deHex2Sul1, Hex2HexNAc4deHex1Sul2, NeuAc1Hex2HexNAc3deHex1Sul1, Hex2HexNAc3deHex3Sul1, NeuGc1Hex2HexNAc3deHex1Sul1, NeuGc1Hex2HexNAc3deHex1Sul1, Hex3HexNAc3deHex2Sul1, NeuGc1Hex2HexNAc2deHex2Sul2, NeuGc1Hex3HexNAc2deHex1Sul2, Hex2HexNAc4deHex2Sul1, Hex2HexNAc4deHex2Sul1, Hex2HexNAc4deHex2Sul1, NeuGc1Hex2HexNAc4Sul1, Hex3Hex4deHex1Sul1, Hex2HexNAc3deHex3Sul2, Hex2HexNAc3deHex3Sul2, NeuGc1Hex2HexNAc3deHex1Sul2, NeuGc1Hex2HexNAc3deHex1Sul2, NeuGc1Hex2HexNAc3deHex1Sul2, NeuGc1Hex3HexNAc2deHex2Sul1, NeuAc1Hex2HexNAc4deHex1, Hex2HexNAc4deHex2Sul2, Hex2HexNAc4deHex2Sul2, NeuGc1Hex2HexNAc4deHex1, Hex2HexNAc3deHex4Sul1, Hex3HexNAc4deHex1Sul2, NeuGc1Hex2HexNAc3deHex2Sul1, NeuGc1Hex2HexNAc3deHex2Sul1, NeuGc1Hex3HexNAc3deHex1Sul1, NeuGc1Hex2HexNAc2deHex3Sul2, Hex2HexNAc4deHex3Sul1, NeuGc1Hex2HexNAc4deHex1Sul1, NeuGc1Hex2HexNAc4deHex1Sul1, NeuGc1Hex2HexNAc4deHex1Sul1, NeuGc1Hex2HexNAc4deHex1Sul1, NeuGc1Hex2HexNAc3deHex2Sul2, Hex3HexNAc3deHex3Sul2, NeuGc1Hex3HexNAc3deHex1Sul2, Hex2HexNAc5deHex2Sul1, Hex2HexNAc4deHex3Sul2, NeuGc1Hex2HexNAc4deHex2Sul1, NeuGc1Hex2HexNAc4deHex2Sul1, NeuGc1Hex3HexNAc4deHex1Sul1, Hex2HexNAc5deHex3Sul1, NeuGc1Hex2HexNAc4deHex2Sul2, NeuGc1Hex2HexNAc4deHex2Sul2, NeuGc1Hex2HexNAc4deHex2Sul2, NeuGc1Hex3HexNAc4deHex1Sul2, Hex3HexNAc4deHex4Sul2, NeuAc1Hex5HexNAc4deHex1, NeuAc1Hex4HexNAc4deHex2Sul1, NeuGc1Hex3HexNAc4deHex4Sul1, NeuGc1Hex4HexNAc4deHex3Sul1, and NeuGc1Hex3HexNAc4deHex4Sul2. In some embodiments, the composition comprises, or alternatively consists essentially of, or yet further consists of at least one, two or all three of NeuAc1HexNAc1, NeuAc2Hex1HexNAc1, and NeuAc1Hex1HexNAc1.

In some embodiments, the sialic acid content of the composition is greater than 5%, 10%, 15%, 20%, 22.5%, 25%, 27.5%, 30%, 32.5%, 35%, or 40% (w/w). In some embodiments, the sialic acid content of the composition is greater than 25% (w/w) and less than 50% (e.g., 25, 30, 35, 40, 45, or 49%). In some embodiments, sialic acid content is determined by LC-MS/MS.

In some embodiments, the composition comprises, or alternatively consists essentially of, or yet further consists of glycopeptide-bound oligosaccharides having at least 7, 14, 21 or all 28 of the following structures: Galβ1-3GalNAc, GlcNAcβ1-6GalNAc, NeuAcα2-6GalNAc, NeuGcα2-6GalNAc, Fucα1-2Galβ1-3GalNAc, Gal+GlcNAcβ1-6GalNAc, Galβ1-3(GlcNAcβ1-6)GalNAc, Galβ1-3GlcNAcβ1-6GalNAc, Galβ1-3(GlcNAcβ1-6)GalNAc, Galβ1-3(6SGlcNAcβ1-6)GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, GlcNAc-(NeuAcα2-6)GalNAc, GalNAc-(NeuAcα2-6)GalNAc, HexNAc-(NeuGcα2-6)GalNAc, Fucα1-2(GalNAcβ1-3)Galβ1-3GalNAc, Fucα1-2Galβ1-4GlcNAcβ1-6GalNAc, Fucα1-2Galβ1-3(GlcNAcβ1-6)GalNAc, Fucα1-2Galβ1-3(6S-GlcNAcβ1-6)GalNAc, Fucα1-2Galβ1-3(NeuAcβ2-6)GalNAc, GlcNAcβ1-3[Galβ1-4(6S)GlcNAcβ1-6]GalNAc, Galβ1-4GlcNAcβ1-3[(6S)GlcNAcβ1-6]GalNAc, Galβ1-3(Fucα1-2Galβ1-4GlcNAcβ1-6)GalNAc, Fucα1-2Galβ1-4(6S)GlcNAcβ1-6[GlcNAcβ1-3]GalNAc, GlcNAcβ1-3[Fucα1-2Galβ1-3(6S-)GlcNAcβ1-6]GalNAc, and Fucα1-2Galβ1-3[Fucα1-2Galβ1-4(6S)GlcNAcβ1-6]GalNAc.

In some embodiments, the composition comprises, or alternatively consists essentially of, or yet further consists of at least 1, 2, 3, 4, 5, 6, 7, 8, 9, or all 10 sialylated glycopeptide-bound oligosaccharide selected from the following: NeuAcα2-6GalNAc, NeuGcα2-6GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, GlcNAc-(NeuAcα2-6)GalNAc, GalNAc-(NeuAcα2-6)GalNAc, HexNAc-(NeuGcα2-6)GalNAc, and Fucα1-2Galβ1-3(NeuAcβ2-6)GalNAc. In some embodiments, the composition comprises, or alternatively consists essentially of, or yet further consists of NeuAcα2-3Galβ1-3GalNAc (Sialyl-T antigen), NeuAcα2-6GalNAc (Sialyl Tn antigen), and NeuAcα2-3Galβ1-3(NeuAcα2-6)GalNAc (Disialyl T antigen).

### EXAMPLES

### Example 1—Preparation of Glycopeptide Composition (GPC)

A 3 kDa spiral wound ultrafiltration (UF) membrane module with filtration surface area 2.7 m² was selected and was operated in a pilot installation. Such surface area allowed for a relatively high permeate flow (63-100 L/h) and shortened the experimental running time. The feed solution was made by mixing the powder GNU100 with demineralized water (DW), concentration before diafiltration were 5%, 5.5% and 7.5% (w/v) respectively in three different trials. GNU100 was prepared by the methods disclosed in Examples 1-3 of WO 2020/104486, published May 28, 2020, which is incorporated herein by reference in its entirety.

Briefly, GPC preparation included the following: Partially purified hydrolyzed porcine gastrointestinal tract mucins were obtained from commercial sources and stabilized at a pH of 5.0 using sulfuric acid or sodium hydroxide, as appropriate. The stabilized mucins were then centrifuged at low speed (500 to 10,000×g) to remove large insoluble particles, fats, and lipids. The mucins were then desalinated using a dialysis membrane (Slide-A-Lyzer Dialysis Flask (2K MWCO) ThermoFisher Scientific) and then concentrated by evaporation with a rotary evaporator (Fisher Scientific) with heating to at least 80° C. forming a slurry. The slurry was further processed by partially filtering through a 0.2 mM Polyethersulfone (PES filter (Millipore Sigma) to remove some amino acids and salts, and the retentate collected.

One hundred ml of the retentate was filtrated on Whatman filter paper (diameter 110 mm, pore size 4-7 mm) by suction using a Buchner funnel. About 100 ml of filtrate (brown liquid) was obtained. The solid residue was discarded, and the filtrate dried under rotary evaporator at 50 mbar and 50° C. m 31.8 g. Total yield 318%. Dry substance yield=88% to yield a powder composition called GNU 100, The powder composition was white to yellow with a neutral or slight amino acid smell and had a 2-5% moisture content. The water solubility of the powder was 80 to 120 g/L at 25° C.

Alternatively, GPC preparation included the following: Partially purified hydrolyzed porcine gastrointestinal tract mucins were obtained from commercial sources and stabilized at a pH of 5.0 using sulfuric acid or sodium hydroxide, as appropriate. The stabilized mucins were then centrifuged at low speed (500 to 10,000×g) to remove large insoluble particles, fats, and lipids. The mucins were desalinated using a dialysis membrane (Slide-A-Lyzer Dialysis Flask (2K MWCO) ThermoFisher Scientific) and then concentrated by evaporation with a rotary evaporator (Fisher Scientific) with heating to at least 80° C., forming a slurry. The slurry was further processed by partially filtering through a 0.2 um (micrometer) Polyethersulfone (PES) filter (Millipore Sigma) to remove some salts and amino acids, and the retentate collected.

One hundred ml of the retentate was filtrated on Whatman filter paper (diameter 110 mm, pore size 4-7 mm) by suction using a Buchner funnel. 100 ml of filtrate (brown liquid) was obtained. The filtrate was then ultra-filtrated though a PES membrane having a molecular weight cutoff (MWCO) of 2 kDa (Millipore Sigma) and the retentate collected. The retentate was then dried under rotary evaporator at 50 mbar and 50° C.

Alternatively, GPC preparation included the following: Partially purified hydrolyzed porcine gastrointestinal tract mucins were obtained from commercial sources and stabilized at a pH of 5.0 using sulfuric acid or sodium hydroxide, as appropriate. The stabilized mucins were then centrifuged at low speed (5,000 to 10,000×g) to remove large insoluble particles and lipids. The mucins were then desalinated using a dialysis membrane (Slide-A-Lyzer Dialysis Flask (2K MWCO) ThermoFisher Scientific) and then concentrated by evaporation with a rotary evaporator (Fisher Scientific) with heating to at least 80° C., forming a slurry. The slurry was further purified by filtering through a 0.2 um Polyethersulfone (PES) filter (Millipore Sigma) and the retentate collected.

One hundred ml of the retentate was filtrated on Whatman filter paper (diameter 110 mm, pore size 4-7 mm) by suction using a Buchner funnel. 100 ml of filtrate (brown liquid) was obtained. The filtrate was then concentrated.

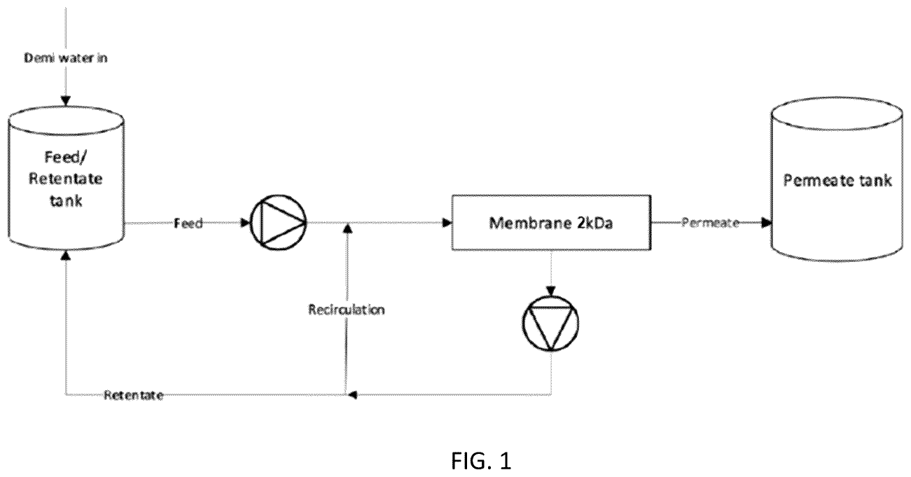

The GPC was further processed as follows: Operating parameters were kept the same for each trial, in which feed flowrate was 450 L/h at a feed pressure fixed at 3 bar. The system is equipped with an internal recirculation pump which can provide 1.05 m³/h crossflow through the membrane surface for less membrane fouling. Retentate was returned to the feed tank for further processing. DW was continuously added into the feed tank with the same flowrate as the permeate. This diafiltration (DF) concept facilitates to ‘wash out’ the unwanted small MW ions and substances for purifying the target proteins, as well as to maintain a stable permeate flow within a trial. Up to 10 cycles of DF was performed for each trial, the conductivity was reduced from 10.47 mS/cm (based on a 7.5% w/v diafiltration starting concentration) to 0.57 mS/cm in the 80 L feed/retentate tank. Afterwards, filtration continued without adding DW in, for a purpose of concentrating the retentate. The trial with highest diafiltration starting concentration 7.5% (w/v) demonstrated the highest product yield, in the end of this trial, approximately 6 L retentate with conductivity of 2.14 mS/cm was obtained, which has a dry product content of 240 g (on a basis of 6 kg powder as feed). A protocol for diafiltration is shown in FIG. 1.

The resulting GPC was found to have the following structures:

| Mass^{[1]} | Composition^{[2]} | Putative structures ^{[3]} |
|---|---|---|
| 384 | Hex1HexNAc1 | Galβ1-3GalNAcol |
| 425-1 | HexNAc2 | GlcNAcβ1-3GalNAcol |
| 425-2 | HexNAc2 | GalNAcα1-3GalNAcol |
| 462 | Hex1HexNAc1Sul | SGalβ1-GlcNAc |
| 464 | Hex1HexNAc1Sul | 3SGalβ1-3GalNAcol |
| 505-1 | HexNAc2Sul1 | 6SGlcNAcβ1-6GalNAcol |
| 505-2 | HexNAc2Sul1 | 6SGlcNAcβ1-3GalNAcol |
| 513 | NeuAc1HexNAc1 | NeuAcα2-6GalNAcol |
| 529 | NeuGc1HexNAc1 | NeuGcα2-6GalNAcol |
| 530-1 | Hex1HexNAc1deHex1 | Fucα1-2(GalNAcα1-3)Galol |
| 530-2 | Hex1HexNAc1deHex1 | Fucα1-2Galβ1-4GlcNAcol |
| 530-3 | Hex1HexNAc1deHex1 | Fucα1-2Galβ1-3GalNAcol |
| 546 | Hex2HexNAc1 | Galβ1-4GlcNAcβ1-3Galol |
| 587-1 | Hex1HexNAc2 | Galβ1-3(GlcNAcβ1- |
| | | 6)GalNAcol |
| 587-2 | Hex1HexNAc2 | Galβ1-4GlcNAcβ1- |
| | | 3GalNAcol |
| 608 | Hex1HexNAc1deHex1Sul1 | 6SGal(Fuc)GlcNAc |
| 626 | Hex2HexNAc1Sul1 | 3SGal-GlcNAcβ1-3Galol |
| 628 | HexNAc3 | GlcNAcβ1-3(GlcNAcβ1- |
| | | 6)GalNAcol |
| 667-1 | Hex1HexNAc2Sul1 | Galβ1-3(6SGlcNAcβ1- |
| | | 6)GalNAcol |
| 667-2 | Hex1HexNAc2Sul1 | 3SGalβ1-3GlcNAcβ1- |
| | | 3GalNAcol |
| 675-1 | NeuAc1Hex1HexNAc1 | Galβ1-3(NeuAcα2- |
| | | 6)GalNAcol |
| 675-2 | NeuAc1Hex1HexNAc1 | NeuAcα2-3Galβ1-3GalNAcol |
| 691-1 | NeuGc1Hex1HexNAc1 | Galβ1-3(NeuGcα2- |
| | | 6)GalNAcol |
| 691-2 | NeuGc1Hex1HexNAc1 | NeuGcα2-3Galβ1-3GalNAcol |
| 692 | Hex2HexNAc1deHex1 | Fucα1-2Galβ-4GlcNAcβ1- |
| | | 3Galol |
| 708-1 | HexNAc3Sul1 | 6SGlcNAcβ1-3(GlcNAcβ1- |
| | | 6)GalNAcol |
| 708-2 | HexNAc3Sul1 | GlcNAcβ1-3(6S-GlcNAcβ1- |
| | | 6)GalNAcol |
| 716-1 | NeuAc1HexNAc2 | GlcNAcβ1-3(NeuAcα2- |
| | | 6)GalNAcol |
| 716-2 | NeuAc1HexNAc2 | GalNAcα1-3(NeuAcα2- |
| | | 6)GalNAcol |
| 732-1 | NeuGc1HexNAc2 | GlcNAcβ1-3(NeuGcα2- |
| | | 6)GalNAcol |
| 732-2 | NeuGc1HexNAc2 | GalNAcα1-3(NeuGcα2- |
| | | 6)GalNAcol |
| 733-1 | Hex1HexNAc2deHex1 | Galβ1-3(Fucα1-4)GlcNAcβ1- |
| | | 3GalNAcol |
| 733-2 | Hex1HexNAc2deHex1 | Fucα1-2(GalNAcα1-3)Galβ1- |
| | | 3GalNAcol |
| 733-3 | Hex1HexNAc2deHex1 | Fucα1-2Galβ1-3GlcNAcβ1- |
| | | 3GalNAcol |
| 733-4 | Hex1HexNAc2deHex1 | Fucα1-2Galβ1-3(GlcNAcβ1- |
| | | 6)GalNAcol |
| 749-1 | Hex2HexNAc2 | Galβ1-3(Galβ1-4GlcNAcβ1- |
| | | 6)GalNAcol |
| 749-2 | Hex2HexNAc2 | Galβ1-3GlcNAcβ1-3Galβ1- |
| | | 3GalNAcol |
| 754 | Hex1HexNAc1deHex2Sul1 | Fucα1-2Gal(Fuc)(6S)GlcNAc |
| 772 | Hex2HexNAc1deHex1Sul1 | 6SGalβ1-4(Fucα1- |
| | | 3)GlcNAcβ1-3Galol |
| 790 | Hex1HexNAc3 | GlcNAcβ1-3(Galβ1- |
| | | 4GlcNAcβ1-6)GalNAcol |
| 813-1 | Hex1HexNAc2deHex1Sul1 | 6SGalβ1-4(Fucα1- |
| | | 3)GlcNAcβ1-3GalNAcol |
| 813-2 | Hex1HexNAc2deHex1Sul1 | Fucα1-2Galβ1-3(6S- |
| | | GlcNAcβ1-6)GalNAcol |
| 821 | NeuAc1Hex1HexNAc1deHex1 | Fucα1-2Galβ1-3(NeuAcα2- |
| | | 6)GalNAcol |
| 829-1 | Hex2HexNAcSul1 | Galβ1-3[Galβ1- |
| | | 4(6S)GlcNAcβ1-6]GalNAcol |
| 829-2 | Hex2HexNAcSul1 | 3SGalβ1-3GlcNAcβ1- |
| | | 3Galβ1-3GalNAcol |
| 837 | NeuGc1Hex1HexNAc1deHex1 | Fucα1-2Galβ1-3(NeuGcα2- |
| | | 6)GalNAcol |
| 870-1 | Hex1HexNAc3Sul1 | GlcNAcα1-4Galβ1- |
| | | 3[(6S)GlcNAcβ1-6]GalNAcol |
| 870-2 | Hex1HexNAc3Sul1 | GlcNAcα1-4(S)Galβ1- |
| | | 4GlcNAcβ1-3GalNAcol |
| 870-3 | Hex1HexNAc3Sul1 | GlcNAcβ1-3[Gal- |
| | | (6S)GlcNAcβ1-6]GalNAcol |
| 870-4 | Hex1HexNAc3Sul1 | Galβ1-4GlcNAcβ1- |
| | | 3(6SGlcNAcβ1-6)GalNAcol |
| 870-5 | Hex1HexNAc3Sul1 | GlcNAcβ1-3[Galβ1- |
| | | 4(6S)GlcNAcβ1-6]GalNAcol |
| 878-1 | NeuAc1Hex1HexNAc2 | NeuAcα2-3(GalNAcβ1- |
| | | 4)Galβ1-3GalNAcol |
| 878-2 | NeuAc1Hex1HexNAc2 | GlcNAca1-4Galβ1- |
| | | 3(NeuAcα2-6)GalNAcol |
| 878-3 | NeuAc1Hex1HexNAc2 | NeuAcα2-3Galβ1- |
| | | 3(GlcNAcβ1-6)GalNAcol |
| 894-1 | NeuGc1Hex1HexNAc2 | GalNAcβ1-4(NeuGcα2- |
| | | 3)Galβ1-3GalNAcol |
| 894-2 | NeuGc1Hex1HexNAc2 | NeuGcα2-3Galβ1- |
| | | 3(GlcNAcβ1-6)GalNAcol |
| 895-1 | Hex2HexNAc2deHex1 | Galβ1-3[Galβ1-3(Fucα1- |
| | | 4)GlcNAcβ1-6]GalNAcol |
| 895-2 | Hex2HexNAc2deHex1 | Galβ1-3(Fucα1-2Galβ1- |
| | | 4GlcNAcβ1-6)GalNAcol |
| 895-3 | Hex2HexNAc2deHex1 | Fucα1-2Galβ1-3(Galβ1- |
| | | 4GlcNAcβ1-6)GalNAcol |
| 911-1 | Hex3HexNAc2 | Galβ1-3(Galα1-3Galβ1- |
| | | 4GlcNAcβ1-6)GalNAcol |
| 911-2 | Hex3HexNAc2 | Galα1-3Galβ1-4GlcNAcβ1- |
| | | 3Galβ1-3GalNAcol |
| 915 | NeuGc1Hex1HexNAc1deHex1Sul1 | NeuGcα2-3(6S)Gal- |
| | | (Fuc)GlcNAc |
| 936-1 | Hex1HexNAc3deHex1 | GlcNAcβ1-3[Gal- |
| | | (Fuc)GlcNAcβ1-6]GalNAcol |
| 936-2 | Hex1HexNAc3deHex1 | Fucα1-2(GalNAcα1-3)Galβ1- |
| | | 4GlcNAcβ1-3GalNAcol |
| 936-3 | Hex1HexNAc3deHex1 | GlcNAcβ1-3(Fucα1-2Galβ1- |
| | | 4GlcNAcβ1-6)GalNAcol |
| 950 | Hex1HexNAc3Sul2 | 3SGalβ1-4GlcNAcβ1- |
| | | 3(6SGlcNAcβ1-6)GalNAcol |
| 952 | Hex2HexNAc3 | GlcNAcα1-4Galβ1-3(Galβ1- |
| | | 4GlcNAcβ1-6)GalNAcol |
| 958 | NeuAc1Hex1HexNAc2Sul1 | NeuAcα2-3Galβ1- |
| | | 3[(6S)GlcNAcβ1-6]GalNAcol |
| 966 | NeuAc2Hex1HexNAc1 | NeuAcα2-3Galβ1- |
| | | 3(NeuAcα2-6)GalNAcol |
| 974 | NeuGc1Hex1HexNAc2Sul1 | NeuGcα2-6Galβ1- |
| | | 3(6SGlcNAcβ1-6)GalNAcol |
| 975-1 | Hex2HexNAc2deHex1Sul1 | Galβ1-3[Galβ1-4(Fucα1- |
| | | 3)(6S)GlcNAcβ1-6]GalNAcol |
| 975-2 | Hex2HexNAc2deHex1Sul1 | Galβ1-3[Fucα1-2(6S)Galβ1- |
| | | 4GlcNAcβ1-6]GalNAcol |
| 975-3 | Hex2HexNAc2deHex1Sul1 | Gal(Fuc)(6S)GlcNAcβ1- |
| | | 3Galβ1-3GalNAcol |
| 975-4 | Hex2HexNAc2deHex1Sul1 | Galβ1-3[Fucα1-2Galβ1- |
| | | 4(6S)GlcNAcβ1-6]GalNAcol |
| 975-5 | Hex2HexNAc2deHex1Sul1 | Fucα1-2Galβ1-3[Galβ1- |
| | | 4(6S)GlcNAcβ1-6]GalNAcol |
| 982 | NeuAc1NeuGc1Hex1HexNAc1 | NeuAcα2-3Galβ1- |
| | | 3(NeuGcα2-6)GalNAcol |
| 998 | NeuGc2Hex1HexNAc1 | NeuGcα2-3Galβ1- |
| | | 3(NeuGcα2-6)GalNAcol |
| 1016-1 | Hex1HexNAc3deHex1Sul1 | GlcNAcβ1-3[Galβ1-4(Fucα1- |
| | | 3)(6S)GlcNAcβ1-6]GalNAcol |
| 1016-2 | Hex1HexNAc3deHex1Sul1 | Gal(Fuc)GlcNAcβ1- |
| | | 3(6SGlcNAcβ1-6)GalNAcol |
| 1016-3 | Hex1HexNAc3deHex1Sul1 | GlcNAcβ1-3[SGalβ1- |
| | | 4(Fucα1-3)GlcNAcβ1- |
| | | 6]GalNAcol |
| 1016-4 | Hex1HexNAc3deHex1Sul1 | Fucα1-2Galβ1-4GlcNAcβ- |
| | | 3(6SGlcNAcβ1-6)GalNAcol |
| 1016-5 | Hex1HexNAc3deHex1Sul1 | GlcNAcβ1-3[Fucα1-2Galβ1- |
| | | 4(6S)GlcNAcβ1-6]GalNAcol |
| 1016-6 | Hex1HexNAc3deHex1Sul1 | GalNAcα1-3(Fucα1-2)Galβ1- |
| | | 3(6SGlcNAcβ1-6)GalNAcol |
| 1016-7 | Hex1HexNAc3deHex1Sul1 | Fucα1-2Galβ1- |
| | | 4(6S)GlcNAcβ1- |
| | | 3(GlcNAcβ1-6)GalNAcol |
| 1016-8 | Hex1HexNAc3deHex1Sul1 | Fucα1-2Galβ1-4GlcNAcβ- |
| | | 3(6SGlcNAcβ1-6)GalNAcol |
| 1024-1 | NeuAc1Hex1HexNAc2deHex1 | Fucα1-2Galβ1-3GlcNAcβ1- |
| | | 3(NeuAcα2-6)GalNAcol |
| 1024-2 | NeuAc1Hex1HexNAc2deHex1 | Fucα1-2Galβ1-4GlcNAcβ1- |
| | | 3(NeuAcα2-6)GalNAcol |
| 1032 | Hex2HexNAc3Sul1 | Galβ1-4GlcNAcβ1-3[Galβ1- |
| | | 4(6S)GlcNAcβ1-6]GalNAcol |
| 1040 | NeuGc1Hex1HexNAc2deHex1 | Fucα1-2Galβ1-4GlcNAcβ1- |
| | | 3(NeuGcα2-6)GalNAcol |
| 1040 | NeuAc1Hex2HexNAc2 | NeuAcα2-3Galβ1-3(Galβ1- |
| | | 4GlcNAcβ1-6)GalNAcol |
| 1041-1 | Hex2HexNAc2deHex2 | Galβ1-3[Fucα1- |
| | | 2Gal(Fuc)GlcNAcβ1- |
| | | 6]GalNAcol |
| 1041-2 | Hex2HexNAc2deHex2 | Fucα1-2Galβ1-3(Fucα1- |
| | | 2Galβ1-4GlcNAcβ1- |
| | | 6)GalNAcol |
| 1055-1 | Hex2HexNAc2deHex1Sul2 | Galβ1-3[3SGal- |
| | | (Fuc)(6S)GlcNAcβ1- |
| | | 6]GalNAcol |
| 1055-2 | Hex2HexNAc2deHex1Sul2 | SGal-(Fuc)(6S)GlcNAc- |
| | | Galβ1-3GalNAcol |
| 1056 | NeuGc1Hex2HexNAc2 | NeuGcα2-3Galβ1-3(Galβ1- |
| | | 4GlcNAcβ1-6)GalNAcol |
| 1079 | NeuGc1Hex2HexNAc1deHex1Sul1 | |
| 1081 | NeuAc1Hex1HexNAc3 | NeuAcα2-3(GalNAcβ1- |
| | | 4)Galβ1-3(GlcNAcβ1- |
| | | 6)GalNAcol |
| 1096-1 | Hex1HexNAc3deHex1Sul2 | 6SGalβ1-(Fucα1-)GlcNAcβ1- |
| | | 3(6SGlcNAcβ1-6)GalNAcol |
| 1096-2 | Hex1HexNAc3deHex1Sul2 | 6SGalβ1-(Fucα1- |
| | | )(6S)GlcNAcβ1- |
| | | 3(GlcNAcβ1-6)GalNAcol |
| 1097 | NeuGc1Hex1HexNAc3 | NeuGcα2-3(GalNAcβ1- |
| | | 4)Galβ1-3(GlcNAcβ1- |
| | | 6)GalNAcol |
| 1098 | Hex2HexNAc3deHex1 | Galβ1-3[Fucα1-2(GalNAcα1- |
| | | 3)Galβ1-4GlcNAcβ1- |
| | | 6]GalNAcol |
| 1105 | Hex1HexNAc2deHex3Sul1 | |
| 1112-1 | Hex2HexNAc3Sul2 | 3SGal-GlcNAcβ1- |
| | | 3(6SGlcNAcβ1-6)Galβ1- |
| | | 3GalNAcol |
| 1112-2 | Hex2HexNAc3Sul2 | SGalβ1-4GlcNAcβ1- |
| | | 3(SGalβ1-3GlcNAcβ1- |
| | | 6)GalNAcol |
| 1120 | NeuAc1Hex2HexNAc2Sul1 | NeuAcα2-3Galβ1-3[Galβ1- |
| | | 4(6S)GlcNAcβ1-6]GalNAcol |
| 1121-1 | Hex2HexNAc2deHex2Sul1 | Galβ1-3[Fucα1-2Galβ1- |
| | | (Fucα−)(6S)GlcNAcβ1- |
| | | 6]GalNAcol |
| 1121-2 | Hex2HexNAc2deHex2Sul1 | Fucα1-2Galβ1-3[Gal- |
| | | (Fuc)(6S)GlcNAcβ1- |
| | | 6]GalNAcol |
| 1121-3 | Hex2HexNAc2deHex2Sul1 | Fucα1-2Galβ1-3[Fucα1- |
| | | 2Galβ1-4(6S)GlcNAcβ1- |
| | | 6]GalNAcol |
| 1136-1 | NeuGc1Hex2HexNAc2Sul1 | NeuGcα2-3Galβ1-3[Galβ1- |
| | | 4(6S)GlcNAcβ1-6]GalNAcol |
| 1136-2 | NeuGc1Hex2HexNAc2Sul1 | NeuGcα2-3Galβ1-3(3SGal- |
| | | GlcNAcβ1-6)GalNAcol |
| 1139 | Hex1HexNAc4deHex1 | GlcNAcβ1-3[GalNAcα1- |
| | | 3(Fucα1-2)Galβ1- |
| | | 3GlcNAcβ1-6]GalNAcol |
| 1161 | NeuAc1Hex1HexNAc3Sul1 | NeuAcα2-3(GalNAcβ1- |
| | | 4)Galβ1-3(6SGlcNAcβ1- |
| | | 6)GalNAcol |
| 1162-1 | Hex1HexNAc3deHex2Sul1 | GlcNAcβ1-3[Fucα1-2Galβ1- |
| | | (Fucα1−)(6S)GlcNAcβ1- |
| | | 6]GalNAcol |
| 1162-2 | Hex1HexNAc3deHex2Sul1 | 6SGlcNAcβ1-3[Fucα1- |
| | | 2Galβ1-(Fucα1−)GlcNAcβ1- |
| | | 6]GalNAcol |
| 1169 | NeuAc2Hex1HexNAc2 | NeuAcα2-3(GalNAcβ1- |
| | | 4)Galβ1-3(NeuAcα2- |
| | | 6)GalNAcol |
| 1177 | NeuGc1Hex1HexNAc3Sul1 | GlcNAcβ1-3[NeuGcα2- |
| | | 3Galβ1-4(6S)GlcNAcβ1- |
| | | 6]GalNAcol |
| 1178-1 | Hex2HexNAc3deHex1Sul1 | Galβ1-3[GalNAcα1-3(Fucα1- |
| | | 2)Galβ1-4(6S)GlcNAcβ1- |
| | | 6]GalNAcol |
| 1178-2 | Hex2HexNAc3deHex1Sul1 | Galβ1-3GlcNAcβ1-3[Fucα1- |
| | | 2Galβ1-4(6S)GlcNAcβ1- |
| | | 6]GalNAcol |
| 1178-3 | Hex2HexNAc3deHex1Sul1 | Galβ1-4GlcNAcβ1-3[Fucα1- |
| | | 2Galβ1-3(6S)GlcNAcβ1- |
| | | 6]GalNAcol |
| 1178-4 | Hex2HexNAc3deHex1Sul1 | Fucα1-2Galβ1-4GlcNAcβ1- |
| | | 3[Galβ1-4(6S)GlcNAcβ1- |
| | | 6]GalNAcol |
| 1185 | NeuAc1NeuGc1Hex1HexNAc2 | NeuAcα2-3(GalNAcβ1- |
| | | 4)Galβ1-3(NeuGcα2- |
| | | 6)GalNAcol |
| 1186 | NeuAc1Hex2HexNAc2deHex1 | NeuAcα2-3Galβ1-3(Fucα1- |
| | | 2Galβ1-4GlcNAcβ1- |
| | | 6)GalNAcol |
| 1201 | NeuGc2Hex1HexNAc2 | NeuGcα2-3(GalNAcβ1- |
| | | 4)Galβ1-3(NeuGcα2- |
| | | 6)GalNAcol |
| 1201-1 | Hex2HexNAc2deHex2Sul1 | |
| 1201-2 | Hex2HexNAc2deHex2Sul1 | Fucα1-2Galβ1-3[SGal- |
| | | (Fuc)(6S)GlcNAcβ1- |
| | | 6]GalNAcol |
| 1202 | NeuGc1Hex2HexNAc2deHex1 | NeuGcα2-3Galβ1-3 (Fucα1- |
| | | 2Galβ1-4GlcNAcβ1- |
| | | 6)GalNAcol |
| 1218 | NeuGc1Hex3HexNAc2 | Galα1-3Galβ1-3(NeuGcα2- |
| | | 3Gal β1-4GlcNAcβ1- |
| | | 6)GalNAcol |
| 1219-1 | Hex1HexNAc4deHex1Sul1 | GlcNAcβ1-3[GalNAcα1- |
| | | 3(Fucα1-2)Galβ1- |
| | | (6S)GlcNAcβ1-6]GalNAcol |
| 1219-2 | Hex1HexNAc4deHex1Sul1 | GalNAcα1-3(Fucα1-2)Galβ1- |
| | | 3GlcNAcβ1-3(6SGlcNAcβ1- |
| | | 6)GalNAcol |
| 1227 | NeuAc1Hex1HexNAc3deHex1 | GlcNAcα1-4(Fucα1-2)Galβ1- |
| | | 4GlcNAcβ1-3(NeuAcα2- |
| | | 6)GalNAcol |
| 1243 | NeuAc1Hex2HexNAc3 | NeuAcα2-3Galβ1- |
| | | 3[GlcNAcα1-4Galβ1- |
| | | 4GlcNAcβ1-6]GalNAcol |
| 1244-1 | Hex2HexNAc3deHex2 | Galβ1-(Fuc)GlcNAcβ1- |
| | | 3(Fucα1-2Galβ1-4GlcNAcβ1- |
| | | 6)GalNAcol |
| 1244-2 | Hex2HexNAc3deHex2 | Fucα1-2Galβ1-4GlcNAcβ1- |
| | | 3(Fucα1-2Galβ1-4GlcNAcβ1- |
| | | 6)GalNAcol |
| 1258-1 | Hex2HexNAc3deHex1Sul2 | |
| 1258-2 | Hex2HexNAc3deHex1Sul2 | |
| 1258-3 | Hex2HexNAc3deHex1Sul2 | SGal-GlcNAcβ1-6[Fucα1- |
| | | 2Gal-(6S)GlcNAcβ1- |
| | | 6]GalNAcol |
| 1258-4 | Hex2HexNAc3deHex1Sul2 | |
| 1259 | NeuGc1Hex2HexNAc3 | NeuGcα2-3Galβ1- |
| | | 3(GlcNAcα1-4Galβ1- |
| | | 4GlcNAcβ1-6)GalNAcol |
| 1266 | NeuAc1Hex2HexNAc2deHex1Sul1 | NeuAcα2-3Galβ1-3 [Fucα1- |
| | | 2Galβ1-4(6S)GlcNAcβ1- |
| | | 6]GalNAcol |
| 1315-1 | Hex2HexNAc4Sul2 | SGal-GlcNAcβ1- |
| | | 3(GlcNAcβ1-6)Galβ1- |
| | | 3(6SGlcNAcβ1-6)GalNAcol |
| 1315-2 | Hex2HexNAc4Sul2 | |
| 1323 | NeuAc1Hex2HexNAc3Sul1 | NeuAcα2-3(GalNAcβ1- |
| | | 4)Galβ1-3[Galβ1- |
| | | 4(6S)GlcNAcβ1-6]GalNAcol |
| 1324-1 | Hex2HexNAc3deHex2Sul1 | Fucα1-2Galβ1-3[GalNAcα1- |
| | | 3(Fucα1-2)Galβ1- |
| | | 3(6S)GlcNAcβ1-6]GalNAcol |
| 1324-2 | Hex2HexNAc3deHex2Sul1 | Fucα1-2Galβ1-4GlcNAcβ1- |
| | | 3 [Fucα1-2Galβ1- |
| | | 4(6S)GlcNAcβ1-6]GalNAcol |
| 1324-3 | Hex2HexNAc3deHex2Sul1 | Fucα1-2Galβ1-3[GalNAcα1- |
| | | 3(Fucα1-2)Galβ1- |
| | | 4(6S)GlcNAcβ1-6]GalNAcol |
| 1324-4 | Hex2HexNAc3deHex2sul1 | SGal-(Fuc)GlcNAcβ1- |
| | | 3[SGal-(Fuc)GlcNAcβ1- |
| | | 6]GalNAcol |
| 1338 | Hex2HexNAc3deHex1Sul3 | SGal-(Fuc)(6S)GlcNAcβ1- |
| | | 3(3SGal-GlcNAcβ1- |
| | | 6)GalNAcol |
| 1339-1 | NeuGc1Hex2HexNAc3Sul1 | |
| 1339-2 | NeuGc1Hex2HexNAc3Sul1 | |
| 1404-1 | Hex2HexNAc3deHex2Sul2 | Gal(Fuc)(6S)GlcNAcβ1- |
| | | 3(Fucα1-2)Galβ1- |
| | | 3(6SGlcNAcβ1-6)GalNAcol |
| 1404-2 | Hex2HexNAc3deHex2Sul2 | SGal-(Fuc)GlcNAcβ1-3[Gal- |
| | | (Fuc)(6S)GlcNAcβ1- |
| | | 6]GalNAcol |
| 1404-3 | Hex2HexNAc3deHex2Sul2 | SGal-GlcNAcβ1-3[Fucα1- |
| | | 2Gal-(Fuc)(6S)GlcNAcβ1- |
| | | 6]GalNAcol |
| 1404-4 | Hex2HexNAc3deHex2Sul2 | |
| 1405 | NeuGc1Hex2HexNAc3deHex1 | NeuGcα2-3(GlcNAcβ1- |
| | | 4)Galβ1-3 (Fucα1-2Galβ1- |
| | | 4GlcNAcβ1-6)GalNAcol |
| 1419 | NeuGc1Hex2HexNAc3Sul2 | |
| 1420 | Hex3HexNAc3deHex1Sul2 | |
| 1428 | NeuGc1Hex2HexNAc2deHex2Sul1 | NeuGcα2-3Galβ1-3[Fucα1- |
| | | 2Galβ1-3(Fucα1- |
| | | 4)(6S)GlcNAcβ1-6]GalNAcol |
| 1461 | Hex2HexNAc4deHex1Sul2 | SGal-GlcNAcβ1- |
| | | 3[GalNAcα1-3(Fucα1-2)Gal- |
| | | (6S)GlcNAcβ1-6]GalNAcol |
| 1469 | NeuAc1Hex2HexNAc3deHex1Sul1 | NeuAcα2-3Galβ1- |
| | | 3[GalNAcα1-3(Fucα1-2)Gal- |
| | | (6S)GlcNAcβ1-6]GalNAcol |
| 1470-3 | Hex2HexNAc3deHex3Sul1 | Fucα1-2Galβ1- |
| | | 4(6S)GlcNAcβ1-3[Fucα1- |
| | | 2Galβ1-3(Fucα1- |
| | | 4)GlcNAcβ1-6]GalNAcol |
| 1485-1 | NeuGc1Hex2HexNAc3deHex1Sul1 | |
| 1485-2 | NeuGc1Hex2HexNAc3deHex1Sul1 | GlcNAcα1-4Galβ1- |
| | | 3[NeuGcα2-6Gal- |
| | | (Fuc)(6S)GlcNAcβ1- |
| | | 6]GalNAcol |
| 1486 | Hex3HexNAc3deHex2Sul1 | |
| 1508 | NeuGc1Hex2HexNAc2deHex2Sul2 | |
| 1524 | NeuGc1Hex3HexNAc2deHex1Sul2 | |
| 1527-1 | Hex2HexNAc4deHex2Sul1 | GlcNAcα1-4(Fucα1- |
| | | 2)GlcNAcβ1-3[Fucα1- |
| | | 2)Galβ1-4(6S)GlcNAcβ1- |
| | | 6]GalNAcol |
| 1527-2 | Hex2HexNAc4deHex2Sul1 | GalNAcα1-3(Fucα1-2)Gal- |
| | | GlcNAcβ1- |
| | | 3[Gal(Fuc)(6S)GlcNAcβ1- |
| | | 6]GalNAcol |
| 1527-3 | Hex2HexNAc4deHex2Sul1 | Galβ1-3GlcNAcβ1- |
| | | 3[GalNAcα1-3(Fucα1-2)Gal- |
| | | (Fucα1-2)(6S)GlcNAcβ1- |
| | | 6]GalNAcol |
| 1542 | NeuGc1Hex2HexNAc4Sul1 | |
| 1543 | Hex3Hex4deHex1Sul1 | |
| 1550-1 | Hex2HexNAc3deHex3Sul2 | SGal-(Fuc)GlcNAcβ1- |
| | | 3 [Fucα1-2Gal- |
| | | (Fuc)(6S)GlcNAcβ1- |
| | | 6]GalNAcol |
| 1550-2 | Hex2HexNAc3deHex3Sul2 | Fucα1-2Gal-(Fuc)GlcNAcβ1- |
| | | 3[SGal-(Fuc)(6S)GlcNAcβ1- |
| | | 6]GalNAcol |
| 1565-4 | NeuGc1Hex2HexNAc3deHex1Sul2 | |
| 1565-5 | NeuGc1Hex2HexNAc3deHex1Sul2 | |
| 1565-6 | NeuGc1Hex2HexNAc3deHex1Sul2 | |
| 1590-2 | NeuGc1Hex3HexNAc2deHex2Sul1 | |
| 1592 | NeuAc1Hex2HexNAc4deHex1 | GalNAcα1-3(Fucα1-2)Gal- |
| | | GlcNAcβ1-3(NeuAcα2-3Gal- |
| | | GlcNAcβ1-6)GalNAcol |
| 1607-1 | Hex2HexNAc4deHex2Sul2 | SGal-(Fuc)GlcNAcβ1- |
| | | 3[GalNAcα1-3(Fucα1-2)Gal- |
| | | (6S)GlcNAcβ1-6]GalNAcol |
| 1607-2 | Hex2HexNAc4deHex2Sul2 | Gal-(Fuc)(6S)GlcNAcβ1- |
| | | 3[GalNAcα1-3(Fucα1-2)Gal- |
| | | (6S)GlcNAcβ1-6]GalNAcol |
| 1608 | NeuGc1Hex2HexNAc4deHex1 | GalNAcα1-3(Fucα1-2)Gal- |
| | | GlcNAcβ1-3(NeuGcα2-3Gal- |
| | | GlcNAcβ1-6)GalNAcol |
| 1616 | Hex2HexNAc3deHex4Sul1 | Fucα1-2Gal(Fuc)GlcNAcβ1- |
| | | 3 [Fucα1-2Gal- |
| | | (Fuc)(6S)GlcNAcβ1- |
| | | 6]GalNAcol |
| 1623-2 | Hex3HexNAc4deHex1Sul2 | Gal-(6S)GlcNAcβ1-3[SGal- |
| | | (Fuc)(6S)GlcNAcβ1-6]Galβ1- |
| | | 3(GlcNAcβ1-6)GalNAcol |
| 1631-1 | NeuGc1Hex2HexNAc3deHex2Sul1 | |
| 1631-2 | NeuGc1Hex2HexNAc3deHex2Sul1 | |
| 1647-2 | NeuGc1Hex3HexNAc3deHex1Sul1 | |
| 1654 | NeuGc1Hex2HexNAc2deHex3Sul2 | |
| 1673-2 | Hex2HexNAc4deHex3Sul1 | |
| 1688-1 | NeuGc1Hex2HexNAc4deHex1Sul1 | |
| 1688-2 | NeuGc1Hex2HexNAc4deHex1Sul1 | |
| 1688-3 | NeuGc1Hex2HexNAc4deHex1Sul1 | |
| 1688-4 | NeuGc1Hex2HexNAc4deHex1Sul1 | |
| 1711 | NeuGc1Hex2HexNAc3deHex2Sul2 | |
| 1712 | Hex3HexNAc3deHex3Sul2 | Fucα1-2Gal(Fuc)GlcNAc- |
| | | (6S)Galβ1-3[6SGal- |
| | | (Fuc)GlcNAcβ1-6]GalNAcol |
| 1727 | NeuGc1Hex3HexNAc3deHex1Sul2 | |
| 1730 | Hex2HexNAc5deHex2Sul1 | GalNAcα1-3(Fucα1-2)Galβ1- |
| | | 4GlcNAcβ1-3[GalNAcα1- |
| | | 3(Fucα1-2)Galβ1- |
| | | 4(6S)GlcNAcβ1-6]GalNAcol |
| 1753 | Hex2HexNAc4deHex3Sul2 | |
| 1834-1 | NeuGc1Hex2HexNAc4deHex2Sul1 | |
| 1834-2 | NeuGc1Hex2HexNAc4deHex2Sul1 | |
| 1850 | NeuGc1Hex3HexNAc4deHex1Sul1 | |
| 1876 | Hex2HexNAc5deHex3Sul1 | GalNAcα1-3(Fucα1-2)Gal- |
| | | GlcNAcβ1-3[GalNAcα1- |
| | | 3(Fucα1-2)Gal- |
| | | (Fuc)(6S)GlcNAcβ1- |
| | | 6]GalNAcol |
| 1914-1 | NeuGc1Hex2HexNAc4deHex2Sul2 | |
| 1914-2 | NeuGc1Hex2HexNAc4deHex2Sul2 | |
| 1914-3 | NeuGc1Hex2HexNAc4deHex2Sul2 | |
| 1930 | NeuGc1Hex3HexNAc4deHex1Sul2 | |
| 2061 | Hex3HexNAc4deHex4Sul2 | |
| 2078 | NeuAc1Hex5HexNAc4deHex1 | NeuAcα2-Galβ1-4GlcNAcβ1- |
| | | 2Manα1-3(Galβ1- |
| | | 4GlcNAcβ1-2Manα1- |
| | | 6)Manβ1-4GlcNAcβ1- |
| | | 4(Fucα1-6)GlcNAcol |
| 2142 | NeuAc1Hex4HexNAc4deHex2Sul1 | |
| 2288 | NeuGc1Hex3HexNAc4deHex4Sul1 | |
| 2304-2 | NeuGc1Hex4HexNAc4deHex3Sul1 | |
| 2368 | NeuGc1Hex3HexNAc4deHex4Sul2 | Fucα1-2Galβ1-3(Fucα1- |
| | | 4)GlcNAcβ1-3[NeuGcα2- |
| | | 6(3S)Galβ1-GlcNAcβ1- |
| | | 3(Fucα1-2)Gal- |
| | | (Fuc)(6S)GlcNAcβ1- |
| | | 6]GalNAcol |

^{[1]}Mass of structure in negative ion mode; mass with hyphens indicates the isomeric structures.
^{[2]}Hex, hexose; HexNAc, N-acetylhexosamine; deHex, fucose; NeuAc, N-acetylneuraminic acid; NeuGc, N-acetyl glycolylneuraminic acid; S, sulphate.
^{[3]} Structures are given in the text according following rules: the structure is described clockwise and left-to-right where reducing end locates rightmost side; “+” is used for uncertain location.

In some embodiments, the oligosaccharides/glycans in the glycopeptide composition was found to comprise the following formulae: Hex1HexNAc1, HexNAc2, Hex1HexNAc1Sul, Hex1NAc2Sul1, NeuAc1HexNAc1, NeuGc1HexNAc1, Hex1HexNAc1deHex1, Hex2HexNAc1, Hex1HexNAc2, HexNAc3, Hex1HexNAc2Sul1, NeuAc1Hex1HexNAc1, NeuGc1Hex1HexNAc1, Hex2HexNAc1deHex1, Hex1NAc3Sul1, NeuAc1HexNAc2, NeuGc1HexNAc2, Hex1HexNAc2deHex1, Hex2HexNAc2, Hex1HexNAc3, Hex1HexNAc2deHex1Sul1, NeuAc1Hex1HexNAc1deHex1, Hex2HexNAcSul1, NeuGc1Hex1HexNAc1deHex1, Hex1HexNAc3Sul1, NeuAc1Hex1HexNAc2, NeuGc1Hex1HexNAc2, Hex2HexNAc2deHex1, Hex3HexNAc2, Hex1HexNAc3deHex1, Hex2HexNAc3, NeuAc1Hex1HexNAc2Sul1, NeuAc2Hex1HexNAc1, NeuGc1Hex1HexNAc2Sul1, Hex2HexNAc2deHex1Sul1, NeuAc1NeuGc1Hex1HexNAc1, NeuGc2Hex1HexNAc1, Hex1HexNAc3deHex1Sul1, NeuAc1Hex1HexNAc2deHex1, Hex2HexNAc3Sul1, NeuGc1Hex1HexNAc2deHex1, NeuAc1Hex2HexNAc2, Hex2HexNAc2deHex2, NeuGc1Hex2HexNAc2, Hex2HexNAc3deHex1, NeuAc1Hex2HexNAc2Sul1, Hex2HexNAc2deHex2Sul1, NeuGc1Hex2HexNAc2Sul1, Hex1HexNAc4deHex1, NeuAc1Hex1HexNAc3Sul1, Hex1HexNAc3deHex2Sul1, NeuAc2Hex1HexNAc2, NeuGc1Hex1HexNAc3Sul1, Hex2HexNAc3deHex1Sul1, NeuAc1Hex2HexNAc2deHex1, NeuGc2Hex1HexNAc2, NeuGc1Hex2HexNAc2deHex1, NeuGc1Hex3HexNAc2, Hex1HexNAc4deHex1Sul1, NeuAc1Hex1HexNAc3deHex1, Hex2HexNAc3deHex2 NeuGc1Hex2HexNAc3, NeuAc1Hex2HexNAc2deHex1Sul1, Hex2HexNAc3deHex2Sul1, NeuAc1Hex2HexNAc3deHex1Sul1, NeuGc1Hex2HexNAc3deHex1Sul1, Hex2HexNAc4deHex2Sul1, Hex2HexNAc5deHex2Sul1, NeuAc1Hex5HexNAc4deHex1, and NeuAc1Hex4HexNAc4deHex2Sul1.

### O-Glycan Analysis Methodology

The glycans were desalted using 1 ml DOWEX® (AG50W-X8), which were packed to a SPE cartridge (Strata X), pre-washed with 2×1 ml methanol, 2×1 ml 1 M HCl and 2×1 ml water. After applying the sample, both the flowthrough and 3×0.5 ml water washout were selected and freeze-dried. The borate was removed by evaporation with 5×200 ul methanol.

O-glycans were released by beta-elimination. Two different ways to remove salts were used: dialysis and DOWEX® ion-exchange chromatography. Dialysis result was only to give an estimate amount of released glycans.

After desalting, the sample was re-constituted in 50 ul of water and analyzed by LC-MS/MS. Glycans were analysed by liquid chromatograph-electrospray ionization tandem mass spectrometry (LC-ESI/MS). The oligosaccharides were separated on a column (10 cm×250 μm) packed in-house with 5 μm porous graphite particles (HYPERCARP™, Thermo-Hypersil, Runcorn, UK). The oligosaccharides were injected on to the column and eluted with an acetonitrile gradient (Buffer A, 10 mM ammonium bicarbonate; Buffer B, 10 mM ammonium bicarbonate in 80% acetonitrile). The gradient (0-45% Buffer B) was eluted for 46 min, followed by a wash step with 100% Buffer B, and equilibrated with Buffer A in next 24 min. A 40 cm×50 μm i.d. fused silica capillary was used as transfer line to the ion source. DOWEX® right after beta-elimination without dialysis. DOWEX® AG50W-X8 is a cation exchange resin which is used to remove sodium in the reaction buffer. The borate is removed by repetitive evaporation with methanol. PGM O-glycans are most neutral and some negatively charged. So, they do not bind to DOWEX®. The samples were analyzed in negative ion mode on a LTQ linear ion trap mass spectrometer (Thermo Electron, San José, CA), with an IonMax standard ESI source equipped with a stainless steel needle kept at −3.5 kV. Compressed air was used as nebulizer gas. The heated capillary was kept at 270° C., and the capillary voltage was −50 kV. In order to reach the number of glycans (>100), two mass ranges (m/z 380-1190 and m/z 1190-2000) were performed (two microscan, maximum 100 ms, target value of 30,000) followed by data-dependent MS2 scans (two microscans, maximum 100 ms, target value of 10,000) with normalized collision energy of 35%, isolation window of 2.5 units, activation q=0.25 and activation time 30 ms). The threshold for MS2 was set to 200 counts. Data acquisition and processing were conducted with Xcalibur software (Version 2.0.7). The LC-MS/MS data was processed using Progenesis QI (Nonlinear Dynamics, Waters).

### Example 2: Structure and Physical Properties of GPC

To elicit a targeted and effective immune response against the dynamic and multivalent aberrant glycosylation of epithelial tumors, a mixture of glycopeptides was selectively purified from a source rich in O-glycosylated long-peptides into the glycoprotein composition as described herein, GPC. GPC is composed of peptide-conjugated glycans weighing ˜2-5 kDa containing high (>21% w/w) glycan content.

### Glycan Content of GPC

| | Glycan content | | | | | |
|---|---|---|---|---|---|---|
| | | Galac- | Glucos- | | | |
| | Fucose | tosamine | amine | Galactose | Glucose | Mannose |
| w/w % | 1.4% | 3.7% | 3.8% | 9.8% | n.a. | 2.3% |

| | |
|---|---|
| Total | (Fucose + GalNAc + GlcNAc + Galacose + |
| | Glucose + Mannose) 21% |

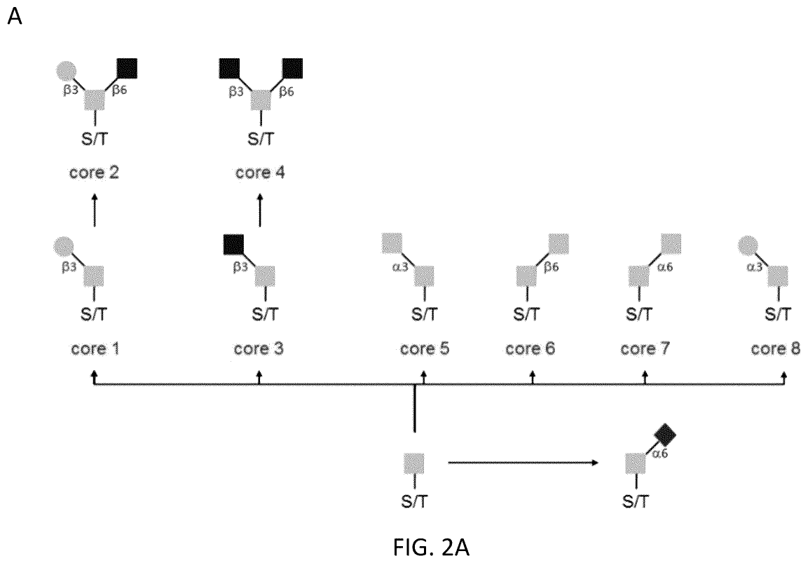

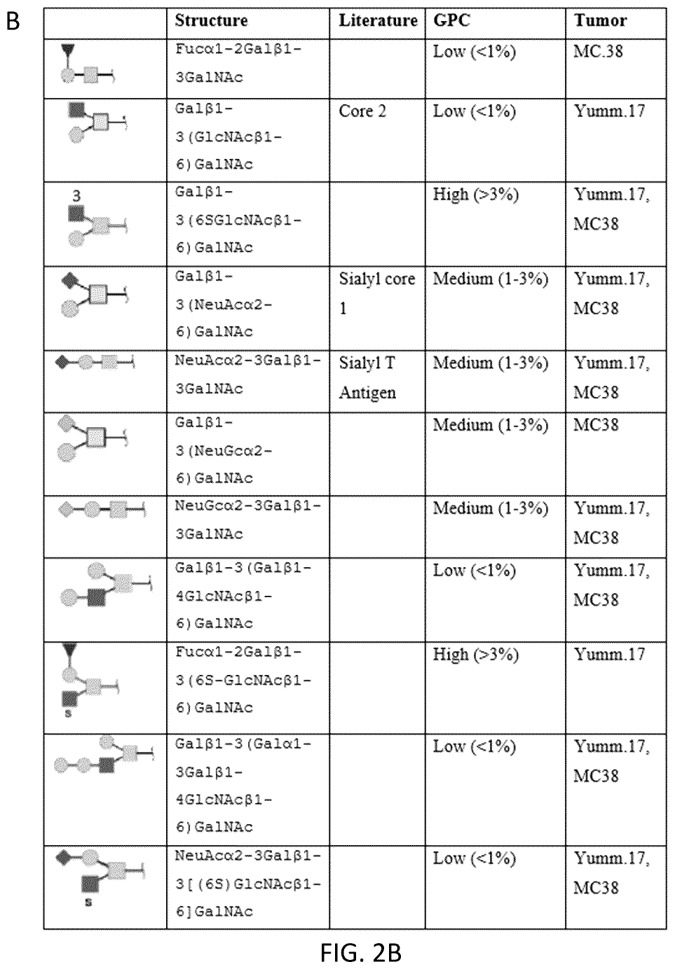

Detailed LC-MS/MS analysis confirmed that these included sT and sialylated core 1 canonical tumor glycan compositions and overall 28.6% sialylation. In-depth comparison of the therapeutic glycopeptides to the MC.38 and YUMM1.7 tumor models identifies high similarities in truncated core-1 and core-2 glycans as well as 9 and 11 unique glycan-epitopes mimicking the respective tumors (FIG. 2B).

The intricate interplay between cellular innate and adaptive responses triggered by glycopeptides has emerged as a fundamental avenue of exploration within cancer immunotherapy. Glycopeptides, such as compounds like GPC, exhibit a unique capacity to mimic the distinctive glycosylation patterns characterizing malignant tumor cells.

### Example 3: Prophylactic Administration of GPC Reduces Tumor Growth

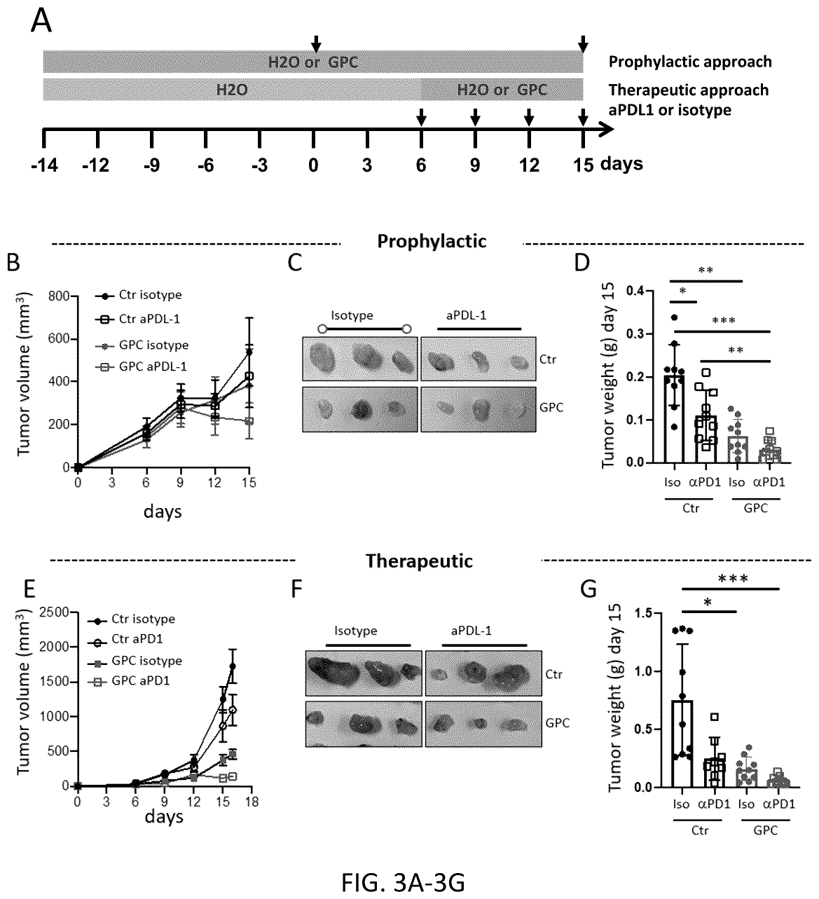

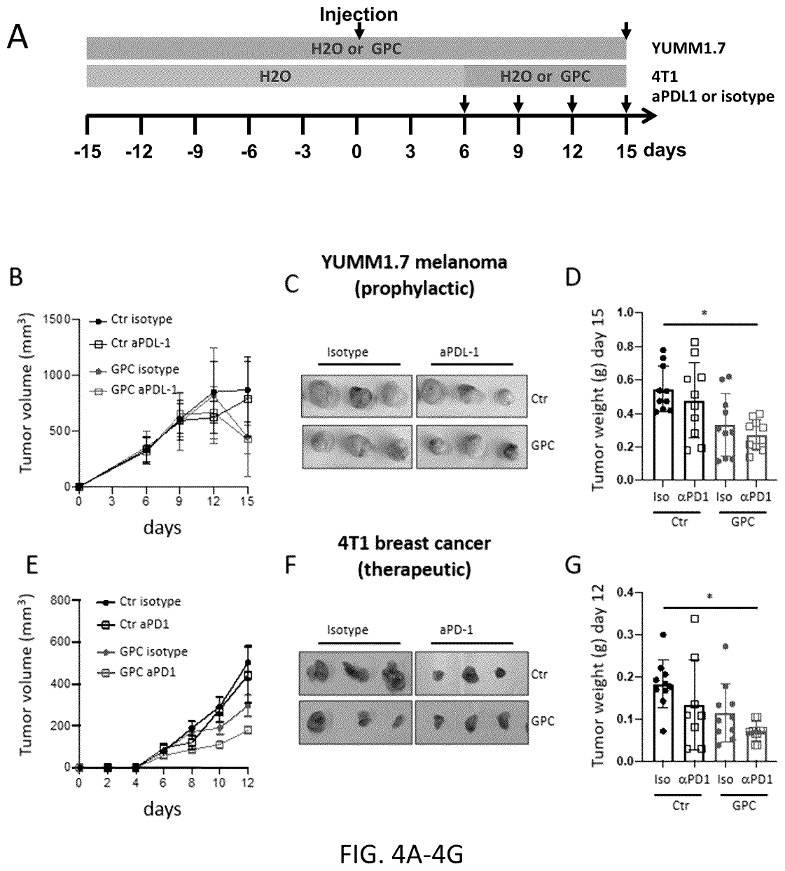

In a first approach to assess whether GPC affects tumor growth, MC38 colorectal cancer cells were injected subcutaneously into the flank of C57BL/6 mice that were treated with 3% GPC dissolved in the drinking water starting two weeks prior to the tumor cell injection. To assess a potential synergistic effect on ICI treatment, the mice were injected with isotype control or anti-PD1 antibody every third day starting 6 days after tumor cell injection (FIG. 3A). Single treatment with anti-PD1 or GPC had limited effects in reducing tumor growth as assessed by tumor size and weight at the end of the experiment (FIGS. 3B-3D). In turn, the combination of anti-PD1 with GPC resulted in clearly reduced tumor growth and reduced tumor weight at the end of the experiment (FIGS. 3B-3D). Similar effects were observed using the YUMM1.7 melanoma model: While anti-PD1 treatment alone had virtually no effect on tumor growth in this model, GPC administration delayed tumor growth and combination of anti-PD1 with GPC resulted in significantly lower tumor volume and tumor weight at the end of the experiment (FIGS. 4A-4D).

### Therapeutic Application of GPC is Effective in Reducing Tumor Growth.

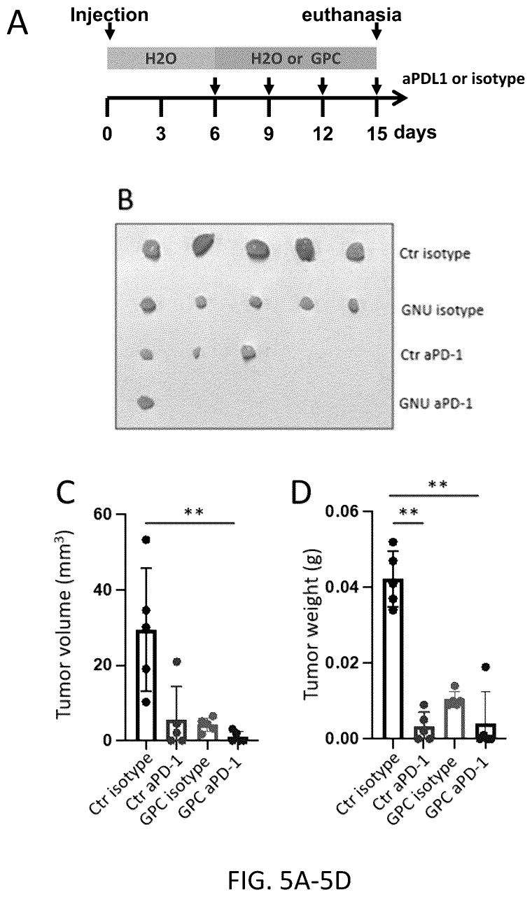

Having shown a beneficial effect of GPC administration in a prophylactic setting, Applicant tested whether GPC administration would also be effective in reducing tumor load in a therapeutic setting, where treatment starts when tumors are already established. For this aim, GPC was administered starting on day 6 post MC38 tumor cell injection, the same day as anti-PD1 treatment (FIG. 3A). Again, the effect of anti-PD1 treatment alone was small, while administration of GPC resulted in significantly slower tumor growth, an effect further enhanced when GPC was combined with anti-PD1 (FIGS. 3E-3G). The therapeutic anti-tumor effect of GPC was also observed when 4T1 breast cancer cells—a cell line that responds poorly to anti-PD1 treatment—were used (FIGS. 4E-4G). In this model, however, significant effects were only observed when GPC was combined with anti-PD1. Taken together, these findings indicate that administration of GPC significantly reduces tumor growth, especially when combined with anti-PD1. Similar effects were observed in an orthotopic tumor model where MC38 cells were injected into the cecum wall, a model that resembles the tumor microenvironment observed in CRC. In this model, anti-PD1 and GPC single treatment both mediated substantial reduction in tumor volume and size, but the combination treatment was more effective than single treatment (FIGS. 5A-5D)

### Example 4: GPC Promotes T Cell Activation

Next, Applicant aimed to unravel the mechanisms how GPC affects anti-tumor immunity. The local cytokine profile, created by cancer-infiltrating immune cells, stromal cells, the tumor-surrounding tissue, and the cancer cells themselves, crucially impacts tumor growth and dictates anti-tumor immune responses. When assessing the cytokine milieu within the tumor tissue from the MC38 therapeutic model, we noted that cytokines that are associated with a more pro-inflammatory immune response (i.e., IL-1b, IL-12, TNFa, IFNg, IL-17) were increased, while regulatory molecules, such as IL-10, and molecules involved in macrophage recruitment (e.g., MCP1, MIP1a) were reduced in GPC treated mice. Despite this reduction in macrophage-recruiting molecules, levels of IL-12p70, a molecule that is predominantly produced by macrophages and dendritic cells and that skews the T cell response towards a pro-inflammatory/cytotoxic response, was significantly enhanced, indicating that there might be a shift in the cytokine production profile in these cells upon GPC administration.

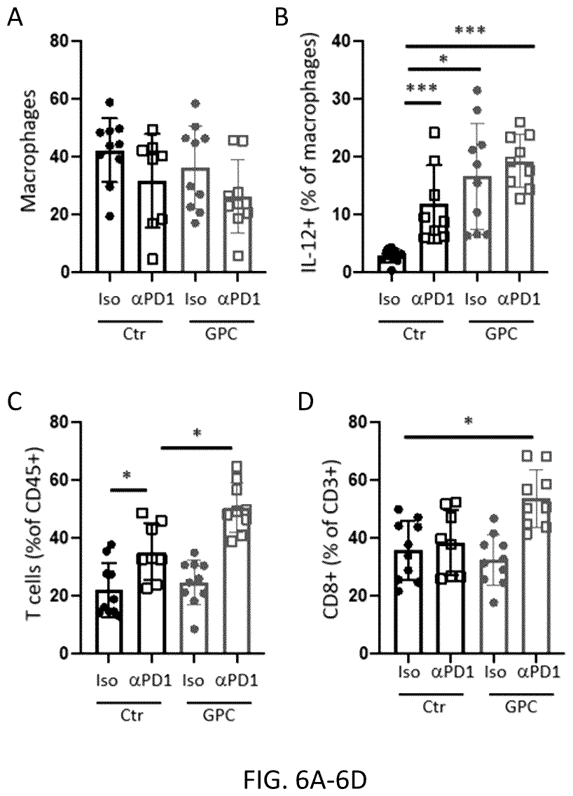

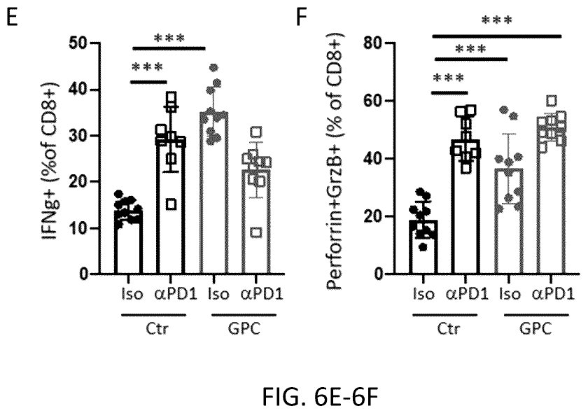

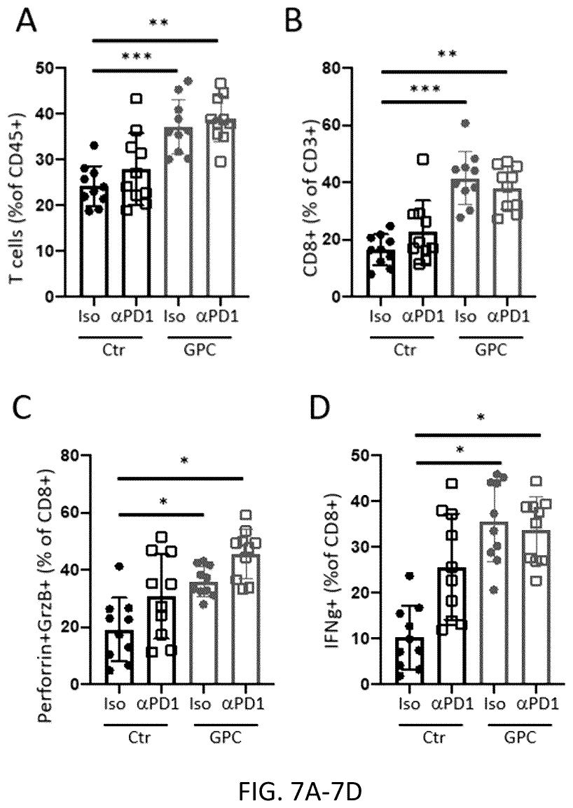

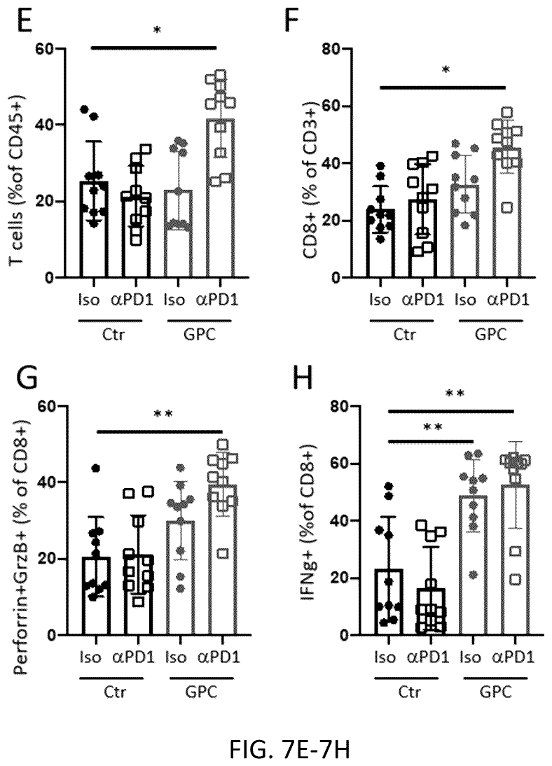

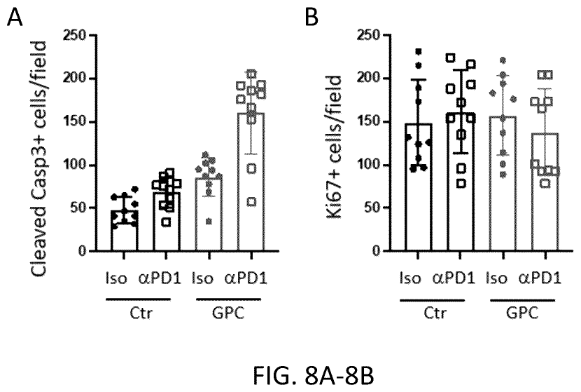

Indeed, flow cytometry analysis of the immune cell landscape in the tumors isolated from mice receiving GPC correlated with changes in the cytokine milieu: while the overall proportion of macrophages among all immune cells was unchanged, the proportion of macrophages that produced IL-12 was significantly higher in mice that received GPC or GPC in combination with anti-PD1 (FIGS. 6A-6B). Proportions of B cells, dendritic cells, and granulocytes were not affected. Notably, the overall proportion of T cells was not affected by GPC treatment alone, but slightly increased following treatment with GPC in combination with anti-PD1 (FIG. 6C). This correlated with the overall abundance of CD8+ T cells that was not affected by GPC treatment alone, but enhanced following treatment with GPC in combination with anti-PD1 (FIG. 6D). Interestingly, however, significantly more CD8+ T cells were positive for IFNg in the GPC only treatment group and the proportion of GranzymeB/Perforin double positive cells was increased by both, GPC treatment alone, and when GPC was combined with anti-PD1 (FIG. 6E-6F) indicating that GPC administration alone already significantly promoted the generation/accumulation of cytotoxic T cells within the tumor tissue. Similar effects were observed on the immune cell landscape in the prophylactic models using the MC38 and YUMM1.7 cells (FIGS. 7A-7G). In line with an increased abundance of cytotoxic T cells, in the tumors from GPC-treated mice, more cells were positive for the apoptosis marker cleaved caspase 3, while the number of Ki67+ (proliferating) cells was unchanged (FIGS. 8A-8B), indicating that the reduced tumor volume is likely not a result of diminished tumor cell growth in response to GPC. Taken together, this suggests that administration of GPC promotes an overall pro-inflammatory immune response in the tumor microenvironment that ultimately leads to reduced tumor growth.

### Example 5: GPC Administration Promotes the Activation of CD8+ T Cells

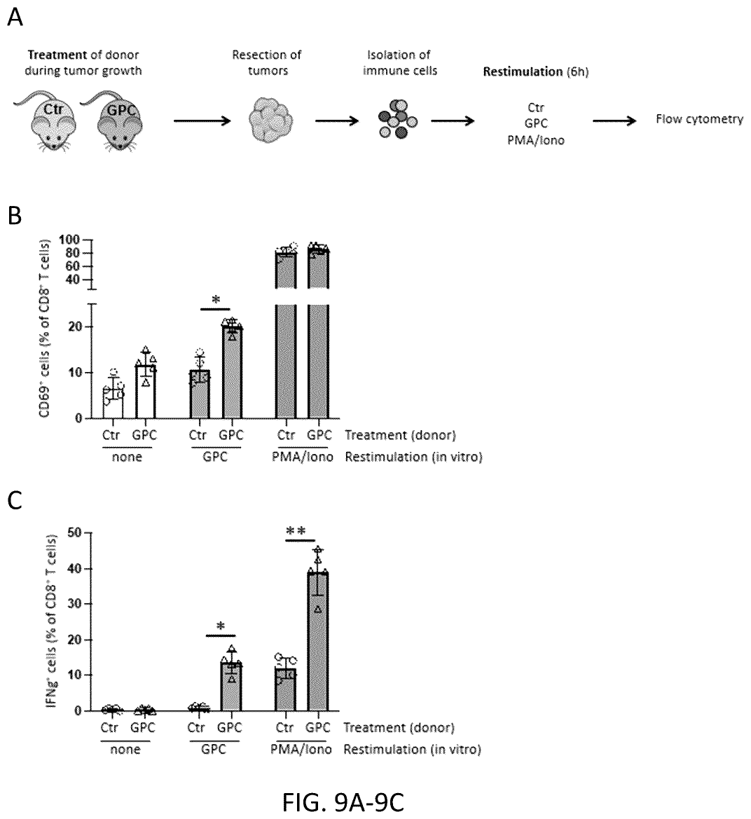

To better understand how GPC administration affects anti-tumor immunity, we tested how immune cells isolated from the tumors of control or GPC-treated mice reacted to in vitro re-stimulation with GPC (FIG. 9A). When immune cells were isolated from the tumors of control or GPC-treated mice, and either left untreated or incubated with GPC, we observed that the percentage of activated T cells was significantly higher in those cells isolated from mice treated with GPC (FIG. 9B). As expected, addition of ionomycin and PMA activated all T cells regardless of the treatment of the donor (FIG. 9B). Of interest, treatment with GPC only provoked a significant IFNγ response in T cells from tumors of mice that had been treated with GPC (FIG. 9C), and ionomycin/PMA activation resulted in higher proportions of IFNγ+ CD8+ T cells in T cells isolated from GPC treated mice (FIG. 3C). This suggests that administration of GPC might promote the expansion or the generation of T cells that react to glycopeptides that are present in our GPC formulation.

### Example 6: Transfer of T Cells from Tumors of GPC-Treated Mice Prevents Tumor Growth

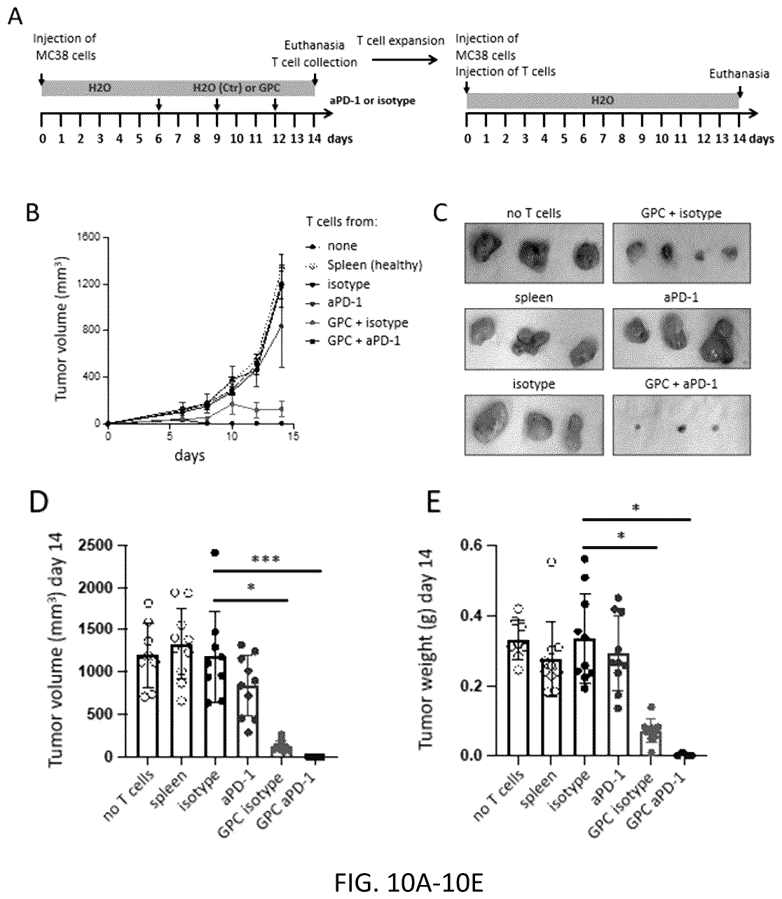

To assess whether T cells mediate the anti-tumor effect of GPC, we performed an experiment where T cells from the tumors of GNU-treated mice were transferred into treatment-naive hosts that were injected with tumors cells at the same day as the T cell transfer (FIG. 10A). T cells from tumors of non-treated mice, or from the spleen of healthy (non-tumor bearing) mice served as control. When T cells were isolated from the tumors of GPC or GPC+anti-PD-1 treated mice, they were able to significantly reduce tumor growth over time (FIG. 10B), resulting in significantly smaller tumors at the end of the experiment, and especially in the mice that received T cells from GPC+anti-PD-1 treated mice, tumor growth were almost completely abolished (FIG. 10C-10E). In contrast, T cells isolated from the spleen of healthy mice, or from tumors of mice that did not receive GPC did not affect tumor growth. There was a small trend towards reduced tumor growth in mice that received T cells from tumors of anti-PD-1-treated mice, however, this effect was not significant. These data demonstrate that T cells from GPC-treated mice mediate an anti-tumor effect and that T cells from GPC or GPC+anti-PD-1 exposed mice are sufficient to reduce tumor growth in mice that were never exposed to GPC themselves.

It has been described that glycans are recognized by dendritic cells, which in turn can process and present those glycan antigens to T cells and promote a glycan-specific T cell responses. Therefore, Applicant hypothesized that the glycans of the present disclosure might promote dendritic cell activation which ultimately leads to enhanced T cell activity. In line with this hypothesis, Applicant observed an upregulation of the maturation and activation markers CD83 and CD86 and increased of IL-6 and TNFa expression after exposure to GPC in monocyte-derived human dendritic cells (Vliet—donor B only 15 μg/ml GPC).

### EQUIVALENTS

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

All publications, patent applications, patents, and other references mentioned herein are expressly incorporated by reference in their entirety, including all formulas and figures, to the same extent as if each were incorporated by reference individually. In case of conflict, the present specification, including definitions, will control.

Other embodiments are set forth within the following claims.

1. A method for treating cancer in a recipient mammal in need thereof comprising administering to the recipient mammal an effective amount of T cells isolated from a donor mammal,
wherein the T cells were prepared by a method comprising administering to the donor mammal an effective amount of a composition comprising a plurality of glycopeptides,
wherein at least 90% of the plurality of glycopeptides in the composition are less than 2 KDa in size, and
wherein the plurality of glycopeptides comprises the following oligosaccharide structures: Fucα1-2Galβ1-3GalNAc, Galβ1-3(GlcNAcβ1-6)GalNAc, Galβ1-3(6SGlcNAcβ1-6)GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, Galβ1-3(Galβ1-4GlcNAcβ1-6)GalNAc, Fucα1-2Galβ1-3(6S-GlcNAcβ1-6)GalNAc, Galβ1-3(Galα1-3Galβ1-4GlcNAcβ1-6)GalNAc, and NeuAcα2-3Galβ1-3[(6S)GlcNAcβ1-6]GalNAc;
thereby treating the cancer in the recipient mammal.

2. The method of claim 1, wherein the T cells are autologous or allogeneic to the recipient mammal, or wherein the T cells are HLA1-deficient T cells or TCR-deficient T cells.

3. The method of claim 1, wherein the composition is administered to the donor mammal for 28 days or more before isolating the T cells.

4. The method of claim 1, wherein the plurality of glycopeptides is derived from porcine gastrointestinal mucins, and wherein the composition is obtained without subjecting the porcine gastrointestinal mucins to conditions or reagents that cause release of oligosaccharides from glycopeptides.

5. The method of claim 1, wherein the composition comprises between about 0.5-1% Fucα1-2Galβ1-3GalNAc containing glycopeptides, between about 0.5-1% Galβ1-3(GlcNAcβ1-6)GalNAc containing glycopeptides, between about 3-5% Galβ1-3(6SGlcNAcβ1-6)GalNAc containing glycopeptides, between about 1-3% Galβ1-3(NeuAcα2-6)GalNAc, between about 1-3% NeuAcα2-3Galβ1-3GalNAc containing glycopeptides, between about 1-3% Galβ1-3(NeuGcα2-6)GalNAc containing glycopeptides, between about 1-3% NeuGcα2-3Galβ1-3GalNAc containing glycopeptides, between about 0.5-1% Galβ1-3(Galβ1-4GlcNAcβ1-6)GalNAc containing glycopeptides, between about 3-5% Fucα1-2Galβ1-3(6S-GlcNAcβ1-6)GalNAc containing glycopeptides, between about 0.5-1% Galβ1-3(Galα1-3Galβ1-4GlcNAcβ1-6)GalNAc containing glycopeptides, and between about 0.5-1% NeuAcα2-3Galβ1-3[(6S)GlcNAcβ1-6]GalNAc containing glycopeptides by weight of the composition.

6. The method of claim 1, wherein the composition has a free glycan content of less than 0.1% by weight, and wherein the composition contains less than 5% of insoluble particles having a diameter greater than 7 m, and wherein the glycopeptides in the plurality of glycopeptides are less than 15 amino acids.

7.-8. (canceled)

9. The method of claim 1, wherein the composition is administered to the donor mammal orally or rectally.

10. The method of claim 1, wherein the cancer is selected from melanoma, breast cancer, lung cancer or colorectal cancer (CRC), or wherein the cancer is stage 3 or 4 cancer, or wherein the cancer is resistant to checkpoint inhibitor therapy.

11.-12. (canceled)

13. The method of claim 1, further comprising administering to the recipient mammal an immune checkpoint inhibitor before, after, or simultaneously with the isolated T cells.

14. A composition comprising isolated T cells and a cryoprotective agent,
wherein the T cells were prepared by a method comprising administering to a donor mammal an effective amount of a composition comprising a plurality of glycopeptides, and
wherein at least 90% of the plurality of glycopeptides in the composition are less than 2 KDa in size, and
wherein the plurality of glycopeptides comprises the following oligosaccharide structures: Fucα1-2Galβ1-3GalNAc, Galβ1-3(GlcNAcβ1-6)GalNAc, Galβ1-3(6SGlcNAcβ1-6)GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, Galβ1-3(Galβ1-4GlcNAcβ1-6)GalNAc, Fucα1-2Galβ1-3(6S-GlcNAcβ1-6)GalNAc, Galβ1-3(Galα1-3Galβ1-4GlcNAcβ1-6)GalNAc, and NeuAcα2-3Galβ1-3[(6S)GlcNAcβ1-6]GalNAc.

15. The composition of claim 14, wherein the composition is administered to the donor mammal for 28 days or more before isolating the T cells, and wherein the T cells are HLA1-deficient T cells or TCR-deficient T cells.

16. The composition of claim 14, wherein the glycopeptides are derived from porcine gastrointestinal mucins, and wherein the composition is obtained without subjecting the porcine gastrointestinal mucins to conditions or reagents that cause complete release of oligosaccharides from glycopeptides.

17. The composition of claim 14, wherein the composition comprises between about 0.5-1% Fucα1-2Galβ1-3GalNAc containing glycopeptides, between about 0.5-1% Galβ1-3(GlcNAcβ1-6)GalNAc containing glycopeptides, between about 3-5% Galβ1-3(6SGlcNAcβ1-6)GalNAc containing glycopeptides, between about 1-3% Galβ1-3(NeuAcα2-6)GalNAc, between about 1-3% NeuAcα2-3Galβ1-3GalNAc containing glycopeptides, between about 1-3% Galβ1-3(NeuGcα2-6)GalNAc containing glycopeptides, between about 1-3% NeuGcα2-3Galβ1-3GalNAc containing glycopeptides, between about 0.5-1% Galβ1-3(Galβ1-4GlcNAcβ1-6)GalNAc containing glycopeptides, between about 3-5% Fucα1-2Galβ1-3(6S-GlcNAcβ1-6)GalNAc containing glycopeptides, between about 0.5-1% Galβ1-3(Galα1-3Galβ1-4GlcNAcβ1-6)GalNAc containing glycopeptides, and between about 0.5-1% NeuAcα2-3Galβ1-3[(6S)GlcNAcβ1-6]GalNAc containing glycopeptides by weight of the composition.

18. The composition of claim 1, wherein the composition has a free glycan content of less than 0.1% by weight, and wherein the composition contains less than 5% of insoluble particles having a diameter greater than 7 μm, and wherein the glycopeptides in the plurality of glycopeptides are less than 15 amino acids.

19.-20. (canceled)

21. The composition of claim 14, wherein the composition is administered to the donor mammal orally or rectally.

22. A method of preparing reprogrammed T cells comprising:
administering to a donor mammal an effective amount of a composition comprising a plurality of glycopeptides,
wherein at least 90% of the plurality of glycopeptides in the composition are less than 2 KDa in size, and
wherein the plurality of glycopeptides comprises the following oligosaccharide structures: Fucα1-2Galβ1-3GalNAc, Galβ1-3(GlcNAcβ1-6)GalNAc, Galβ1-3(6SGlcNAcβ1-6)GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, Galβ1-3(Galβ1-4GlcNAcβ1-6)GalNAc, Fucα1-2Galβ1-3(6S-GlcNAcβ1-6)GalNAc, Galβ1-3(Galα1-3Galβ1-4GlcNAcβ1-6)GalNAc, and NeuAcα2-3Galβ1-3[(6S)GlcNAcβ1-6]GalNAc; and
isolating the T cells from the donor mammal.

23. The method of claim 22, further comprising cryopreserving the isolated T cells.

24. The method of claim 22, wherein the composition is administered to the donor mammal for 28 days or more before isolating the T cells, or wherein the T cells are HLA1-deficient T cells or TCR-deficient T cells.

25. The method of claim 22, wherein the glycopeptides were derived from porcine gastrointestinal mucins, and wherein the composition is obtained without subjecting the porcine gastrointestinal mucins or a partially purified fraction thereof to conditions or reagents that cause complete release of oligosaccharides from glycopeptides.

26. The method of claim 22, wherein the composition comprises between about 0.5-1% Fucα1-2Galβ1-3GalNAc containing glycopeptides, between about 0.5-1% Galβ1-3(GlcNAcβ1-6)GalNAc containing glycopeptides, between about 3-5% Galβ1-3(6SGlcNAcβ1-6)GalNAc containing glycopeptides, between about 1-3% Galβ1-3(NeuAcα2-6)GalNAc, between about 1-3% NeuAcα2-3Galβ1-3GalNAc containing glycopeptides, between about 1-3% Galβ1-3(NeuGcα2-6)GalNAc containing glycopeptides, between about 1-3% NeuGcα2-3Galβ1-3GalNAc containing glycopeptides, between about 0.5-1% Galβ1-3(Galβ1-4GlcNAcβ1-6)GalNAc containing glycopeptides, between about 3-5% Fucα1-2Galβ1-3(6S-GlcNAcβ1-6)GalNAc containing glycopeptides, between about 0.5-1% Galβ1-3(Galα1-3Galβ1-4GlcNAcβ1-6)GalNAc containing glycopeptides, and between about 0.5-1% NeuAcα2-3Galβ1-3[(6S)GlcNAcβ1-6]GalNAc containing glycopeptides by weight of the composition.

27. The method of claim 22, wherein the composition has a free glycan content of less than 0.1% by weight, and wherein the composition contains less than 5% of insoluble particles having a diameter greater than 7 μm, and wherein the glycopeptides in the plurality of glycopeptides are less than 15 amino acids.

28.-30. (canceled)
